(19) United States
(12) Patent Application Publication
(10) Pub. No.: US 2004/0161839 A1
(43) Pub. Date: Aug. 19, 2004

(54) Method to alter sugar moieties

(76) Inventors: Hung-Wen Liu, Austin, TX (US); David H. Sherman, An Arbor, MI (US); Lishan Zhao, Carlsbad, CA (US)

Correspondence Address: Schwegman Lundberg, Woessner & Kluth, PO Box 2938, Minneapolis, MN, 55402, US

(21) Appl. No.: 10/398,605

(22) PCT Filed: Oct. 5, 2001
(86) PCT No.: PCT/US01/31255

(51) Int. Cl. C12N 1/20

(52) U.S. Cl. 435/252.3

(57) ABSTRACT

A method to modify the structure of sugars is provided.

### STATEMENT OF GOVERNMENT RIGHTS

This invention was made with a grant from the Government of the United States of America (grants GM48562, GM35906, GM54346 and GM58196 from the National Institutes of Health). The Government may have certain rights in the invention.

### BACKGROUND OF THE INVENTION

Nature continues to be the inspiration for most pharmaceutical drug leads and given the synthetic challenge posed by many complex secondary metabolites, the emerging field of combinatorial biosynthesis has become a rich new source for modified non-natural scaffolds (Katz et al., 1993; Hutchinson et al., 1995; Carreras et al., 1997; Jacobsen et al., 1997; Cane et al., 1998; Marsden et al., 1997; McDaniel et al., 1999). Yet, many naturally occurring bioactive secondary metabolites, e.g., polyketides, possess unusual carbohydrate ligands which serve as molecular recognition elements critical for biological activity (Omura, 1984; Weymouth-Wilson, 1997). Without these essential sugar attachments, the biological activities of most clinically important secondary metabolites are either completely abolished or dramatically decreased. Glycosyltransferases responsible for the final glycosylation of certain secondary metabolites show a high degree of promiscuity towards the nucleotide sugar donor (Zhao et al., 1998a; Zhao et al., 1998b; Borisova et al., 1999; Weber et al., 1991; Decker et al., 1995, Sasaki et al., 1996; Solenberg et al., 1997; Madduri et al., 1998; Salah-Bey et al., 1998; Gaisseret al., 1998; Wohlert et al., 1998). These discoveries have opened the door to the possibility of manipulating the corresponding biosynthetic pathways for modifying the crucial glycosylation pattern of natural, or non-natural, secondary metabolite scaffolds in a combinatorial fashion. To date, the genetic manipulation of the carbohydrate appendage for any given metabolite has generally been limited to alterations and/or knock-outs of the small subset of genes required to construct and attach that carbohydrate moiety (Madduri et al., 1998; Hutchinson, 1998; Wohlert et al., 1998).

Thus, what is needed is a method to significantly modify or alter the sugar appendage for a particular metabolite.

### SUMMARY OF THE INVENTION

The invention provides a method to alter the sugar structure diversity for a particular metabolite via the recruitment and collaborative action of sugar genes from a variety of sugar biosynthetic pathways to yield a metabolite comprising a non-natural sugar, e.g., a novel glycosylated polyketide. This alteration can be accomplished in vivo through genetic engineering. For example, the method of the invention provides a modified recombinant bacterial host cell that is genetically engineered to produce novel polyketides having non-natural sugar structures. To prepare the modified recombinant host cell of the invention, a sugar biosynthetic gene(s) from a heterologous (e.g., non-native or different) sugar biosynthetic pathway, or one that is modified in vitro and encodes an enzyme having an activity or specificity that is different than the native (wild type) enzyme, is introduced into a recombinant host cell that produces a substrate for the enzyme(s) encoded by that gene(s) to yield a modified recombinant host cell that produces a novel product, i.e., one not produced by the corresponding recombinant host cell. Preferably, the product from the modified recombinant host cell comprises a sugar(s) that is significantly different than the sugar on the naturally occurring product from the corresponding wild type cell, e.g., the sugar on the modified product is not a stereoisomer of the sugar on the naturally occurring product. Also preferably, the recombinant host cell and the modified recombinant host cell are genetically modified so that at least one gene for sugar biosynthesis, for example, in a sugar biosynthetic gene cluster, in that cell is disrupted, e.g., via an insertion or deletion, resulting in the accumulation of an intermediate in the biosynthetic pathway which is disrupted. The disruption may be in a nucleic acid sequence present in the genome of the cell or present in an extrachromosomal element in the cell. Thus, the invention is useful to generate libraries of polyketides and other sugar-containing molecules that are biologically active or can be activated. For example, if the product is an acetylated sugar, a deacetylase may be employed to render the product biologically active. Moreover, the availability of such libraries can greatly decrease the time for drug discovery.

As described hereinbelow, a 4-ketohexose aminotransferase gene (calH) from the calicheamicin pathway of Micromonospora echinospora spp. calichenisis was introduced into a mutant strain of Streptomyces venezuelae in which the 4-dehydrase gene (desI) in the methymycin/pikromycin pathway was deleted. Deletion of desI gene led to the accumulation of 4-keto-6-deoxyglucose intermediate which is the substrate of CalH. Consequently, heterologous expression of calH in this mutant resulted in the production of two methymycin/pikromycin-calicheamicin hybrids. These results not only reinforce the indiscriminate nature of the corresponding glycosyltransferase (DesVII) but also clearly demonstrate the ability to engineer secondary metabolite glycosylation through a rational selection of gene combinations. In addition, the results confirm that the calH gene codes for the TDP-6-deoxy-D-glycero-L-threo-4-hexulose 4-aminotransferase of the calicheamicin pathway.

As also described herein, a significant expansion of sugar structural diversity can be achieved if various L-sugars are incorporated into metabolites such as macrolides. The heterologous expression of selected genes from the L-dihydrostreptose pathway, for example, the strM and strL genes of Streptomyces griseus that encode a 6-deoxy-4-hexulose 3,5-epimerase and a dihydrostreptose synthase, respectively, was accomplished in a S. venezuelae mutant. Growth of the engineered S. venezuelae strain resulted in the accumulation of a set of methymycin/pikromycin analogs, each carrying a L-rhamnose. Formation of these new derivatives confirmed the relaxed substrate specificity of the desosamine glycosyltransferase DesVII, and the feasibility of preparing novel metabolites by reconstitution of a hybrid pathway. In addition, these results provide evidence of the collaborative functions of StrM and StrL, and established the close resemblance of the dihydrostreptose and apiose biosynthetic pathways.

Thus, the invention provides a modified recombinant bacterial host cell comprising at least one nucleic acid segment which encodes at least one sugar biosynthetic enzyme. Preferably, a nucleic acid segment of the invention does not encode a glycosyltransferase or any other non-sugar biosynthetic sequences such as polyketide synthase sequences. The modified recombinant host cell may include more than one nucleic acid segment, each encoding a different enzyme, or one nucleic acid segment encoding one or more enzymes. The modified recombinant host cell also preferably comprises a disrupted nucleic acid sequence, which corresponds to a nucleic acid sequence in a wild type host cell that encodes at least one sugar biosynthetic enzyme from a pathway that is different than the pathway of the enzyme(s) encoded by the nucleic acid segment. For example, the nondisrupted wild type nucleic acid sequence may encode a dehydrase, a reductase, a TDP-sugar synthase, a TDP-sugar dehydratase, an amino transferase, a N-methyltransferase, and/or a tautomerase. The disruption results in the accumulation of a substrate(s) for the enzyme(s) encoded by the nucleic acid segment thus yielding a novel sugar. The modified recombinant host cell also preferably produces a product having the novel sugar linked thereto, e.g., the native (endogenous) glycosyltransferase(s) transfers the novel sugar to another molecule, e.g., a polyketide such as an aglycone, to yield a novel product such as a macrolide. Alternatively, a nucleic acid molecule encoding a glycosyltransferase having relaxed substrate specificity may also be introduced to the recombinant host cell so as to provide an enzyme which attaches the novel sugar to another molecule in the modified recombinant host cell.

Preferred cells for use in the invention include any cell which produces a metabolite such as a polyketide, anticancer agent or antibiotic that has or can be modified to accommodate a sugar. Antibiotic-producing cells include but are not limited to Actinoplanes, Actinomadura, Bacillus, Cephalosporium, Micromonospora, Penicillium, Nocardia, and Streptomyces, which either produce an antibiotic or contains genes which, if expressed, would produce an antibiotic or other biologically active compound, e.g., any cell which contains the genes sno, str; tyl, cay; srm, tet, act, gra tcm, mit/mmc, elm, sal, rif, grs, srf, bac, dau, sty, dnr, sna, fren, avr, ole, urd, ery, or any combination thereof. Examples of actinomycetes that naturally produce polyketides include but are not limited to Micromonospora rosaria, Micromonospora megalomicea, Saccharopolyspora erythraea, Streptomyces antibioticus, Streptomyces albereticuli, Streptomyces ambofaciens, Streptomyces avermitilis, Streptomyces fradiae, Streptomyces griseus, Streptomyces hydroscopicus, Streptomyces tsukulubaensis, Streptomyces mycarofasciens, Streptomyces platenesis, Streptomyces violaceoniger; Streptomyces violaceoniger, Streptomyces thermotolerans, Streptomyces rimosus, Streptomyces peucetius, Streptomyces coelicolor, Streptomyces glaucescens, Streptomyces roseofulvus, Streptomyces cinnamonensis, Streptomyces curacoi, and Amycolatopsis mediterranei. Other examples of polyketide-producing microorganisms that produce polyketides naturally include various Actinomadura, Dactylosporangium and Nocardia strains. Preferred Streptomyces spp. include but are not limited to Streptomyces venezuelae (e.g., ATCC 15439, ATCC 15068, MCRL 0306, SC 2366 or 3629), Streptomyces narbonensis (e.g., ATCC 19790), Streptomyces eurocidicus, Streptomyces zaomyceticus (MCRL 0405), Streptomyces flavochromogens, Streptomyces sp. AM400, Streptomyces felleus, Streptomyces fradiae, Streptomyces argillaceus, Streptomyces olivaceus, Streptomyces peucetius, and Streptomyces griseus.

Moreover, those same cells are a preferred source of the nucleic acid segments of the invention. Thus, any cell which encodes a sugar biosynthetic gene is a source for the nucleic acid segments of the invention. For example, a source for nucleic acid segments are cells which produce a compound having a sugar including but not limited to cells that produce streptomycin, carbomycin, tylosin, spiramycin, streptothricin, erythromycin, vancomycin, teicoplanin, chloroeremycin, methymycin, pikromycin, uramycin, granaticin, oleandomicin, landomycin, tetracenomycin, doxorubicin, mithramycin, epirubicin, and daunoribicin, or other sugar-containing compounds such as calicheamicin or nystatin, are included within the scope of the nucleic acid segments for use in the practice of the invention.

In one embodiment of the invention, a recombinant host cell in which a nucleic acid sequence encoding at least one of the enzymes in desosamine biosynthesis is disrupted so as to alter desosamine synthesis, and is augmented with a nucleic acid segment which encodes a homolog of the enzyme encoded by the nondisrupted form of the nucleic acid sequence, yielding a modified recombinant host cell. In one embodiment, the modified recombinant host cell does not have a disruption is desI and does not consist of a calH nucleic acid segment. A “homolog” of a reference sugar biosynthetic enzyme is an enzyme which can recognize the substrate of the reference biosynthetic enzyme and catalyze a reaction. For example, TylB is a homolog of DesI, CalH is a homolog of DesI, StrL and StrM together are a homolog of DesI, and TylM2 is a homolog of DesVI. Preferred homologs catalyze a reaction that produces a product, such an intermediate in sugar biosynthesis, that is different than the product of the reference enzyme. Homologs can be identified functionally using methods such as those described herein. Generally, a homolog has at least about 28% amino acid sequence identity to the reference enzyme.

Other methods to identify a nucleic acid segment for use in the invention is by hybridization or computer assisted sequence alignments, e.g., using default settings. In one embodiment of the invention, the nucleic acid sequence of the invention hybridizes under low, moderate or stringent hybridization conditions to the nucleic acid segment of the invention. Low, moderate and stringent hybridization conditions are well known to the art, see, for example sections 9.47-9.51 of Sambrook et al. (Molecular Cloning: A Laboratory Manual, Cold Spring Harbor Laboratory, Cold Spring Harbor, N.Y. (1989). For example, stringent conditions are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium, citrate) at 50 to 55° C. Exemplary moderate stringency conditions include hybridization in 40 to 45% formamide, 1.0 M NaCl, 1% SDS at 37° C., and a wash in 0.5× to 1×SSC at 50 to 60° C. Exemplary high stringency conditions include hybridization in 50% formamide, 1 M NaCl, 1% SDS at 37° C., and a wash in 0.1×SSC at 60 to 65° C.

Also provided are methods of preparing the modified recombinant host cells of the invention and methods of using them, e.g., to prepare biologically active products or products which can be modified to a biologically active product.

The invention also provides an isolated and purified nucleic acid segment comprising a nucleic acid sequence comprising a sugar (desosamine) biosynthetic gene cluster, a biologically active variant or fragment thereof, wherein the nucleic acid sequence is not derived from the eryC gene cluster of Saccharopolyspora erythraea. The isolated nucleic acid segment comprising the gene cluster preferably includes a nucleic acid sequence comprising SEQ ID NO:3 (see PCT/US 99/14398, which is incorporated by reference herein), or a fragment or variant thereof. The cluster was found to encode nine polypeptides including DesI (e.g., SEQ ID NO:8 encoded by SEQ ID NO:7), DesII (e.g., SEQ ID NO:10 encoded by SEQ ID NO:9), DesIII (e.g., SEQ ID NO:12 encoded by SEQ ID NO:11), DesIV (e.g., SEQ ID NO:14 encoded by SEQ ID NO:13), DesV (e.g., SEQ ID NO:16 encoded by SEQ ID NO:15), DesVI (e.g., SEQ ID NO:18 encoded by SEQ ID NO:17), DesVII (e.g., SEQ ID NO:20 encoded by SEQ ID NO:19), DesVIII (e.g., SEQ ID NO:22 encoded by SEQ ID NO:21), and DesR (e.g., SEQ ID NO:24 encoded by SEQ ID NO:23) (see FIG. 1). It is also preferred that the nucleic acid segment of the invention encoding DesR is not derived from the eryB gene cluster of Saccharopolyspora erythraea or the oleD gene from Streptomyces antibioticus. Preferably, the nucleic acid segment comprising the desosamine biosynthetic gene cluster hybridizes under moderate, or more preferably stringent, hybridization conditions to SEQ ID NO:3, or a fragment thereof.

The invention also provides a variant polypeptide having at least about 80%, more preferably at least about 90%, and even more preferably at least about 95%, but less than 100%, contiguous amino acid sequence identity to the polypeptide having an amino acid sequence comprising SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, or a fragment thereof. A preferred variant polypeptide, or a subunit or fragment of a polypeptide, of the invention includes a variant or subunit polypeptide having at least about 1%, more preferably at least about 10%, and even more preferably at least about 50%, the activity of the polypeptide having the amino acid sequence comprising SEQ ID NO:8, SEQ ID NO:10, SEQ ID NO:12, SEQ ID NO:14, SEQ ID NO:16, SEQ ID NO:18, SEQ ID NO:20, SEQ ID NO:22, or SEQ ID NO:24. Thus, for example, the glycosyltransferase activity of a polypeptide of SEQ ID NO:20 can be compared to a variant of SEQ ID NO:20 having at least one amino acid substitution, insertion, or deletion relative to SEQ ID NO:20.

A variant nucleic acid sequence of the invention has at least about 80%, more preferably at least about 90%, and even more preferably at least about 95%, but less than 100%, contiguous nucleic acid sequence identity to a nucleic acid sequence comprising SEQ ID NO:3, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, or a fragment thereof.

Also provided is an expression cassette comprising a nucleic acid sequence comprising a desosamine biosynthetic gene cluster, a biologically active variant or fragment thereof operably linked to a promoter functional in a host cell, as well as host cells comprising an expression cassette of the invention. Thus, the expression cassettes of the invention are useful to express individual genes within the cluster, e.g., the desR gene which encodes a glycosidase or the desVII gene which encodes a glycosyltransferase having relaxed substrate specificity for polyketides and deoxysugars, i.e., the glycosyltransferase processes sugar substrates other than TDP-desosamine. Thus, the desVII gene can be employed in combinatorial biology approaches to synthesize a library of macrolide compounds having various polyketide and deoxysugar structures. Moreover, the expression of a glycosylase in a host cell which synthesizes a macrolide antibiotic may be useful in a method to reduce toxicity of, e.g., inactivate, the antibiotic. For example, a host cell which produces the antibiotic is transformed with an expression cassette encoding the glycosyltransferase. The recombinant glycosyltransferase is expressed in an amount that reversibly inactivates the antibiotic. To activate the antibiotic, the antibiotic, preferably the isolated antibiotic which is recovered from the host cell, is contacted with an appropriate native or recombinant glycosidase.

Preferably, the nucleic acid segment encoding desosamine in the expression cassette of the invention is not derived form the eryC gene cluster of Saccharopolyspora erythraea. Preferred host cells are prokaryotic cells, although eukaryotic host cells are also envisioned. These host cells are useful to express desosamine, analogs or derivatives thereof as well as individual polypeptides which can then be isolated from the host cell. Also provided is an expression cassette or host cell comprising antisense sequences from at least a portion of the desosamine biosynthetic gene cluster.

Another embodiment of the invention is a recombinant host cell, e.g., a bacterial cell, in which at least a portion of a nucleic acid sequence encoding desosamine in the host chromosome is disrupted, e.g., deleted or interrupted (e.g., by an insertion) with heterologous sequences, or substituted with a variant nucleic acid sequence of the invention, so as to alter, preferably so as to result in a decrease or lack of, desosamine synthesis and/or so as to result in the synthesis of an analog or derivative of desosamine. Preferably, the nucleic acid sequence which is disrupted is not derived from the eryC gene cluster of Saccharopolyspora erythraea. Thus, the recombinant host cell of the invention has at least one gene, i.e., desI, desII, desIII, desIV, desV, desVI, desVII, desVIII or desR, which is disrupted. One embodiment of the invention includes a recombinant host cell in which the desVI gene, which encodes an N-methyltransferase, is disrupted, for example, by replacement with an antibiotic resistance gene. Preferably, such a host cell produces an aglycone having an N-acetylated aminodeoxy sugar, 10-deoxy-methylonide, a compound of formula (7), a compound of formula (8), or a combination thereof. Thus, the deletion or disruption of the desVI gene may be useful in a method for preparing novel sugars.

Another preferred embodiment of the invention is a recombinant bacterial host cell in which the desR gene, which encodes a glycosidase such as β-glucosidase, is disrupted. Preferably, the host cell synthesizes C-2′β-glucosylated macrolide antibiotics, for example, a compound of formula (13), a compound of formula (14), or a combination thereof. Therefore, the invention further provides a compound of formula (8), (9), (13) or (14). It will be appreciated by those skilled in the art that each atom of the compounds of the invention having a chiral center may exist in and be isolated in optically active and racemic forms. Some compounds may exhibit polymorphism. It is to be understood that the present invention encompasses any racemic, optically active, polymorphic or stereoisomeric form, or mixtures thereof, of a compound of the invention, which possess the useful properties described herein, it being well known in the art how to prepare optically active forms (for example, by resolution of the racemic form by recrystallization techniques, by synthesis from optically active starting materials, by chiral synthesis, or by chromatographic separation using a chiral stationary phase) and how to determine activity using the standard tests described herein, or using other similar tests which are well known in the art.

Also provided is a method for directing the biosynthesis of specific glycosylation-modified polyketides by genetic manipulation of a polyketide-producing microorganism. The method comprises introducing into a polyketide-producing microorganism a DNA sequence encoding enzymes for sugar biosynthesis, e.g., desosamine biosynthesis such as a DNA sequence comprising SEQ ID NO:3, a variant or fragment thereof, so as to yield a microorganism that produces specific glycosylation-modified polyketides. Alternatively, an anti-sense DNA sequence of the invention may be employed. Then the glycosylation-modified polyketides are isolated from the microorganism. It is preferred that the DNA sequence is modified so as to result in the inactivation of at least one enzymatic activity in sugar biosynthesis or in the attachment of the sugar to a polyketide.

The compounds (products) produced by the recombinant host cells and modified recombinant host cells of the invention may be particularly useful as biologically active agents, such as those useful to prepare a medicament for the treatment of a pathological condition or a symptom in a mammal, e.g., a human. Thus, the products include pharmaceuticals such as chemotherapeutic agents, immunosuppressants, agents to treat asthma, chronic obstructive pulmonary disease as well as other diseases involving respiratory inflammation, cholesterol-lowering agents, or macrolide-based antibiotics which are active against a variety of organisms, e.g., bacteria, including multi-drug-resistant pneumococci and other respiratory pathogens, as well as viral and parasitic pathogens; or as crop protection agents (e.g., fungicides or insecticides). Methods employing these compounds, e.g., to treat a mammal, bird or fish in need of such therapy, such as a patient having a bacterial, viral or parasitic infection, cancer, respiratory disease, or in need of immunosuppression, e.g., during cell, tissue or organ transplantation, are also envisioned.

### BRIEF DESCRIPTION OF THE FIGURES

FIG. 1. Schematic diagram of the desosamine biosynthetic pathway and the enzymatic activity associated with each of the desosamine biosynthetic polypeptides.

FIG. 2. Schematic of the conversion of the inactive (diglycosylated) form of methymycin and pikromycin to the active form of methymycin and pikromycin.

FIG. 3. Schematic diagram of the desosamine biosynthetic pathway.

FIG. 4. Pathway for the synthesis of a compound of formula 7 and 8 in desVI⁻ mutants of Streptomyces.

FIG. 5. Structure and biosynthesis of methymycin, pikromycin, and related compounds in Streptomyces venezuelae ATCC 15439. Methymycin: R₁═OH, R₂═H, neomethymycin: R₁═H, R₂═OH; pikromycin: R₃═OH, narbomycin: R₃═H. Polyketide synthase components PikAI, PikAII, PikAIII, PikAIV, and PikAV are represented by solid bars. Each circle represents an enzymatic domain in the Pik PKS system. KS: β-ketoacyl-ACP synthase, AT: acyltransferase, ACP: acyl carrier protein, KR: β-ketoacyl-ACP reductase, DH: β-hydroxyl-thioester dehydratase, ER: enoyl reductase, KS^{Q}: a KS-like domain, KR with a cross: nonfunctional KR, TE: thioesterase domain, and TEII: type II thioesterase. Des represents all eight enzymes for desosamine biosynthesis and transfer and PikC is the cytochrome P450 monooxygenase responsible for hydroxylation at R₁, R₂, and R₃ positions (Xue et al., 1998).

FIG. 6. Organization of the pik cluster in S. venezuelae. Each arrow represents an open reading frame (ORF). The direction of transcription and relative sizes of the ORFs deduced from nucleotide sequence are indicated. The cluster is composed of four genetic loci: pikA, pikB (des), pikC, and pikR. Cosmid clones are denoted as overlapping lines.

FIG. 7. Conversion of YC-17 and narbomycin by PikC P450 hydroxylase.

FIG. 8. Nucleotide sequence (SEQ ID NO:3) and inferred amino acid sequence (SEQ ID NO:4) of the desosamine gene cluster.

FIG. 9. Exemplary and preferred amino acid substitutions.

FIG. 10. Pathway for desosamine biosynthesis.

FIG. 11. Schematic of pathway leading to methymycin/neomethymycin analogs 18 and 19.

FIG. 12. Macrolide having D-quinovose.

FIG. 13. Products produced by desI mutant.

FIG. 14. Macrolides produced in a desI mutant which expresses CalH.

FIG. 15. Natural substrate for and product of CalH, and structure of calicheamicin.

FIG. 16. Macrolides produced in a desI mutant which expresses StrL and StrM.

FIG. 17. Natural substrate for and product of StrL and StrM.

FIG. 18. Substrate for and products of apiose synthase.

FIG. 19. Scheme for desosamine biosynthesis and intermediates in des mutants.

FIG. 20. Alternative scheme for desosamine biosynthesis.

### DETAILED DESCRIPTION OF THE INVENTION

Definitions

As used herein, a “Type I polyketide synthase” is a single polypeptide with a single set of iteratively used active sites. This is in contrast to a Type II polyketide synthase which employs active sites on a series of polypeptides.

As used herein, a “module” is one of a series of repeated units in a multifunctional protein, such as a Type I polyketide synthase or a fatty acid synthase.

As used herein, a “premature termination product” is a product which is produced by a recombinant multifunctional protein which is different than the product produced by the non-recombinant multifunctional protein. In general, the product produced by the recombinant multifunctional protein has fewer acyl groups.

As used herein, a “recombinant” nucleic acid or protein (polypeptide) molecule is a molecule where the nucleic acid molecule which encodes the protein has been modified in vitro, so that its sequence is not naturally occurring, or corresponds to naturally occurring sequences that are not positioned as they would be positioned in a genome which has not been modified.

A “recombinant” host cell of the invention has been genetically manipulated so as to alter, e.g., decrease or disrupt, or, alternatively, increase, the function or activity of at least one gene in a sugar biosynthetic pathway. The manipulation may occur in an extrachromosomal genetic element which comprises the at least one gene or in the genome of the cell. In contrast, a “wild type” or “nonrecombinant” cell has not been genetically manipulated. The genetic manipulation in the recombinant cell preferably results in the absence of a product (compound) that is produced by the corresponding wild type cell or the production of a product that is not produced by the corresponding wild type cell.

A “modified” recombinant host cell of the invention is a recombinant host cell that has been genetically manipulated so as to express at least one isolated nucleic acid segment, preferably in the form of an expression cassette which includes a promoter, that is introduced to the recombinant cell to form the modified recombinant host cell. The genetic manipulation in the modified recombinant host cell preferably results in the production of a product (compound) that is not produced by the corresponding recombinant host cell or the corresponding wild type cell.

As used herein, a DNA that is “derived from” a gene or gene cluster is a DNA that has been isolated and purified in vitro from genomic DNA, or synthetically prepared on the basis of the sequence of genomic DNA.

As used herein, the “pik” or “pik/met” gene cluster includes sequences encoding a polyketide synthase (pikA), desosamine biosynthetic enzymes (pikB, also referred to as des), a cytochrome P450 (pikC), regulatory factors (pikD) and enzymes for cellular self-resistance (pikR).

As used herein, the terms “isolated and/or purified” refer to in vitro isolation of a DNA or polypeptide molecule from its natural cellular environment, and from association with other components of the cell, such as nucleic acid or polypeptide, so that is can be sequenced, replicated and/or expressed. For example, “an isolated DNA molecule encoding an enzyme for desosamine biosynthesis or a fragment thereof” is RNA or DNA containing greater than 7, preferably 15, and more preferably 20 or more sequential nucleotide bases that encode a biologically active polypeptide, fragment, or variant thereof, that is complementary to the non-coding, or complementary to the coding strand, of a RNA encoding at least one enzyme for desosamine biosynthesis, or hybridizes to the RNA or DNA comprising the desosamine biosynthetic gene cluster and remains stably bound under low, moderate or preferably stringent conditions, as defined by methods well known to the art, e.g., in Sambrook et al., 1989.

An “antibiotic” as used herein is a substance produced by a microorganism which, either naturally or with limited chemical modification, will inhibit the growth of or kill another microorganism or eukaryotic cell.

An “antibiotic biosynthetic gene” is a nucleic acid, e.g., DNA, segment or sequence that encodes an enzymatic activity which is necessary for an enzymatic reaction in the process of converting primary metabolites into antibiotics.

An “antibiotic biosynthetic pathway” includes the entire set of antibiotic biosynthetic genes necessary for the process of converting primary metabolites into antibiotics. These genes can be isolated by methods well known to the art, e.g., see U.S. Pat. No. 4,935,340.

Antibiotic-producing organisms include any organism, including, but not limited to, Actinoplanes, Actinomadura, Bacillus, Cephalosporium, Micromonospora, Penicillium, Nocardia, and Streptomyces, which either produces an antibiotic or contains genes which, if expressed, would produce an antibiotic.

An antibiotic resistance-conferring gene is a DNA segment that encodes an enzymatic or other activity which confers resistance to an antibiotic.

The term “polyketide” as used herein refers to a large and diverse class of natural products, including but not limited to -antibiotic, antifungal, anticancer, and anti-helminthic compounds. Polyketides include but are not limited to macrolides, anthracyclines, angucyclins, avermectins, milbemycins, tetracyclines, polyenes, polyethers, ansamycins and isochromanequinones and the like. Polyketide antibiotics include, but are not limited to anthracyclines and macrolides of different types (polyenes and avermectins as well as classical macrolides such as erythiomycins). Macrolides are produced by, for example, S. erytheus, S. autibioticus, S. venezuelae, S. fradiae and S. narbonensis.

The term “glycosylated” in the context of another molecule refers to a molecule that contains one or more sugar residues.

The term “sugar” or “saccharide” refers to a polyhydroxylated aldehyde or ketone. The polyhydroxylated aldehyde or ketone can optionally be linked to lipids, peptides and/or proteins. Sugars may have additional substituents such as amino, sulfate or phosphate groups, in addition to the carbon-hydrogen-oxygen core. A polymer consisting of two to ten saccharide units is termed an oligosaccharide (OS), e.g., monosaccharides, disaccharides, e.g., sucrose, and trisaccharides, and those consisting of more than ten saccharide units is termed a polysaccharide (PS). These monosaccharide building blocks can be linked in at least 10 different ways, leading to an astronomical number of different combinations and permutations. Sugars include, e.g., trioses, pentoses and hexoses, ribose, glucose, as well as deoxy sugars such as fructose, rhamnose, and deoxyribose, and 6-, 2,6-, 3,6-, 4,6-, 2,3,6-deoxysugars, such as olivose, oliose, mycarose, rhodinose, mycinose, and other modified sugars (e.g., amino sugars including mycaminose, desosamine, vancosamine and daunosamine). Additional suitable sugars are disclosed, e.g., in D. Voet, Biochemistry, Wiley: New York, 1990; L. Stryer, Biochemistry, (3rd Ed.), W. H. Freeman and Co.: New York, 1975; J. March, Advanced Organic Chemistry, Reactions, Mechanisms and Structure, (2nd Ed.), McGraw Hill: New York, 1977; F. Carey and R. Sundberg, Advanced Organic Chemistry, Part B: Reactions and Synthesis, (2nd Ed.), Plenum: New York, 1977; and references cited therein). Saccharide derivatives can conveniently be prepared as described in International Patent Applications Publication Numbers WO 96/34005 and 97/03995.

The term “glycosylation-modified” as it relates to a particular molecule refers to a molecule having a changed glycosylation pattern or configuration relative to that particular molecule's unmodified or native state.

The term “polyketide-producing microorganism” as used herein includes any microorganism that can produce a polyketide naturally or after being suitably engineered (i.e., genetically). Examples of actinomycetes that naturally produce polyketides include but are not limited to Micromonospora rosaria, Micromonospora megalomicea, Saccharopolyspora erythraea, Streptomyces antibioticus, Streptomyces albereticuli, Streptomyces ambofaciens, Streptomyces avermitilis, Streptomyces fradiae, Streptomyces griseus, Streptomyces hydroscopicus, Streptomyces tsukulubaensis, Streptomyces mycarofasciens, Streptomyces platenesis, Streptomyces violaceoniger, Streptomyces violaceoniger, Streptomyces thermotolerans, Streptomyces rimosus, Streptomyces peucetius, Streptomyces coelicolor, Streptomyces glaucescens, Streptomyces roseofulvus, Streptomyces cinnamonensis, Streptomyces curacoi, and Amycolatopsis mediterranei. Other examples of polyketide-producing microorganisms that produce polyketides naturally include various Actinomadura, Dactylosporangium and Nocardia strains.

The term “sugar biosynthesis gene” as used herein refers to nucleic acid sequences or segments from organisms such as Micromonospora, Streptomyces venezuelae, Streptomyces fradiae, Streptomyces griseus, Streptomyces peucetius, Streptomyces argillaceous, and Streptomyces olivaceus that encode sugar biosynthesis enzymes, and is intended to include sugar biosynthetic DNA from other polyketide-producing microorganisms.

The term “sugar biosynthesis enzymes” as used herein refers to polypeptides which are involved in the biosynthesis and/or attachment of polyketide-associated sugars and their derivatives and intermediates.

The term “polyketide-associated sugar” refers to a sugar that is known to attach to polyketides or that can be attached to polyketides.

The term “sugar derivative” refers to a sugar which is naturally associated with a polyketide but which is altered relative to the unmodified or native state, including but not limited to N-3-α-desdimethyl D-desosamine.

The term “sugar intermediate” refers to an intermediate compound produced in a sugar biosynthesis pathway.

As used herein, the term “derivative” means that a particular compound (product) produced by a host cell of the invention or prepared in vitro using polypeptides encoded by the nucleic acid molecules of the invention, is modified so that it comprises other moieties, e.g., peptide or polypeptide molecules, such as antibodies or fragments thereof, nucleic acid molecules, sugars, lipids, fats, a detectable signal molecule such as a radioisotope, e.g., gamma emitters, small chemicals, metals, salts, synthetic polymers, e.g., polylactide and polyglycolide, surfactants and glycosaminoglycans, which are covalently or non-covalently attached or linked to the compound.

It will be appreciated by those skilled in the art that each atom of the compounds of the invention having a chiral center may exist in and be isolated in optically active and racemic forms. Some compounds may exhibit polymorphism. It is to be understood that the present invention encompasses any racemic, optically active, polymorphic or stereoisomeric form, or mixtures thereof, of a compound of the invention, which possess the useful properties described herein, it being well known in the art how to prepare optically active forms (for example, by resolution of the racemic form by recrystallization techniques, by synthesis from optically active starting materials, by chiral synthesis, or by chromatographic separation using a chiral stationary phase) and how to determine activity using the standard tests described herein, or using other similar tests which are well known in the art.

The term “sequence homology” or “sequence identity” means the proportion of base matches between two nucleic acid sequences or the proportion amino acid matches between two amino acid sequences. When sequence homology is expressed as a percentage, e.g., 50%, the percentage denotes the proportion of matches over the length of sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

Two amino acid sequences are homologous if there is a partial or complete identity between their sequences and/or have the same or similar activity. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater (Dayhoff, 1972). The two sequences or parts thereof are more preferably homologous as used herein if their amino acids are greater than or equal to 29% identical.

The following terms are used to describe the sequence relationships between two or more polynucleotides or polypeptides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) by the homology alignment algorithm of Needleman and Wunsch (1970), by the search for similarity method of Pearson and Lipman (1988), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. Preferably, default settings are employed to identify homologs using computerized algorithms.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

As applied to polypeptides, the term “substantial identity” or “homology” means that two peptide sequences, when optimally aligned, such as by the programs GAP or BESTFIT using default gap weights, share at least about 29 percent sequence identity, preferably at least about 35 percent sequence identity and/or have the same or similar activity, i.e., recognize one or more common substrate(s) and thereby produce a product.

In accordance with the present invention there is provided a modified recombinant host cell, derived from a recombinant host cell, the genome of which is altered, optionally to disrupt sugar biosynthesis that occurs in the corresponding wild type cell. The modified recombinant host cell is augmented with a nucleic acid segment that encodes at least one sugar biosynthetic enzyme that is a homolog of an enzyme encoded by the wild type cell which is absent or present in a reduced amount in the recombinant host cell as a result of the disruption. Thus, the modified recombinant host cell includes a least one expression cassette comprising at least one isolated and purified nucleic acid segment which encodes a sugar biosynthetic enzyme(s) that recognizes the substrate of an enzyme(s) encoded by the wild type cell and which is not expressed, or expressed in a reduced amount, in the recombinant cell. The enzyme(s) encoded by the nucleic acid segment produces a substrate for another sugar biosynthetic enzyme or for a glycosyltransferase.

The invention described herein can be used for the production of a diverse range of novel compounds including glycosylated polyketides, e.g., antibiotics, through genetic redesign of sugar biosynthetic DNA such as that found in Streptomyces spp. as well as other polyketide producing organisms. This gene allows for the selective production of particular compounds, including the production of novel compounds. For example, combinational biosynthetic-based modification of compounds may be accomplished by selective activation or disruption of specific genes within the sugar gene cluster and expressing other sugar biosynthetic genes into biosynthetic libraries which are assayed for a wide range of biological activities, to derive greater chemical diversity. A further example includes the introduction of biosynthetic gene(s) into a particular host cell so as to result in the production of a novel compound due to the activity of the biosynthetic gene(s) on other metabolites, intermediates or components of the host cells.

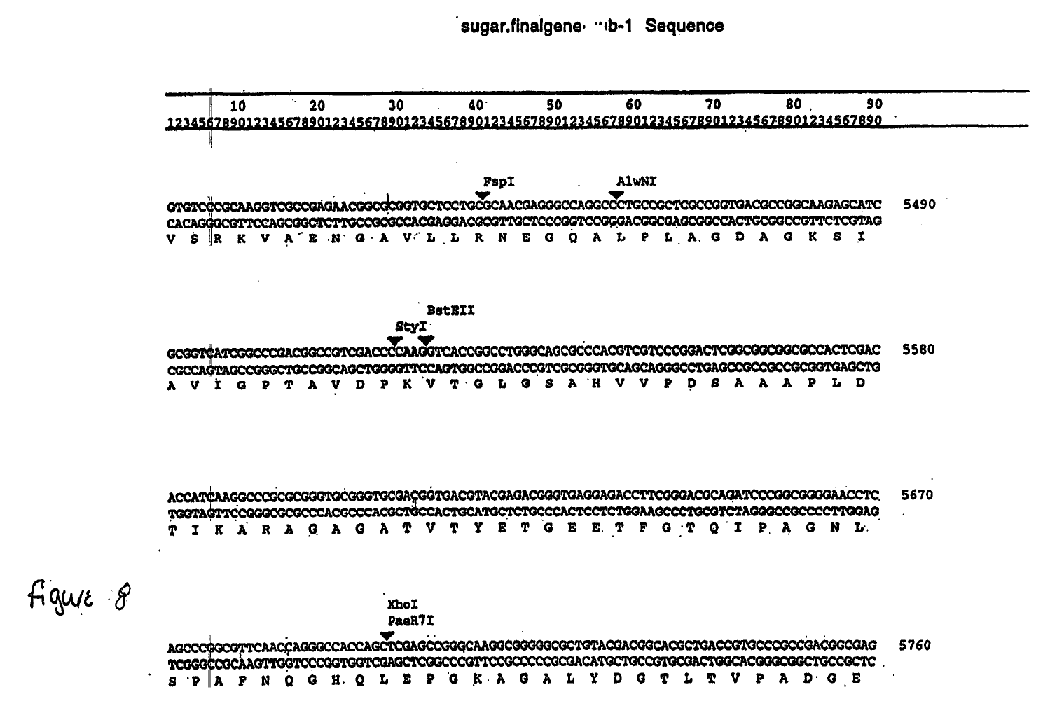

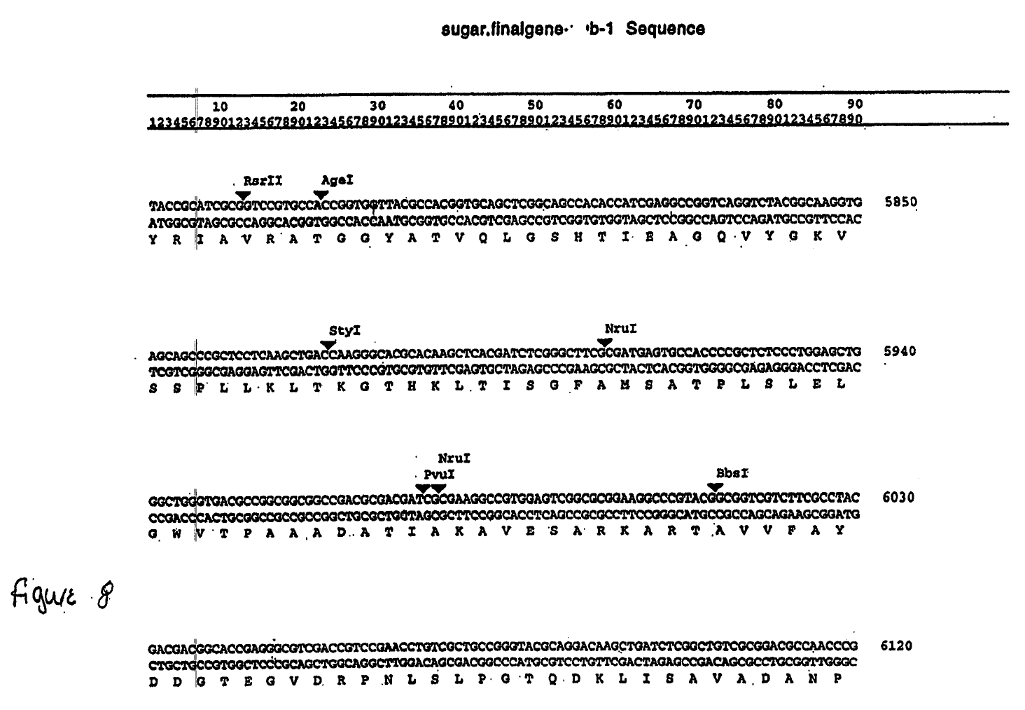

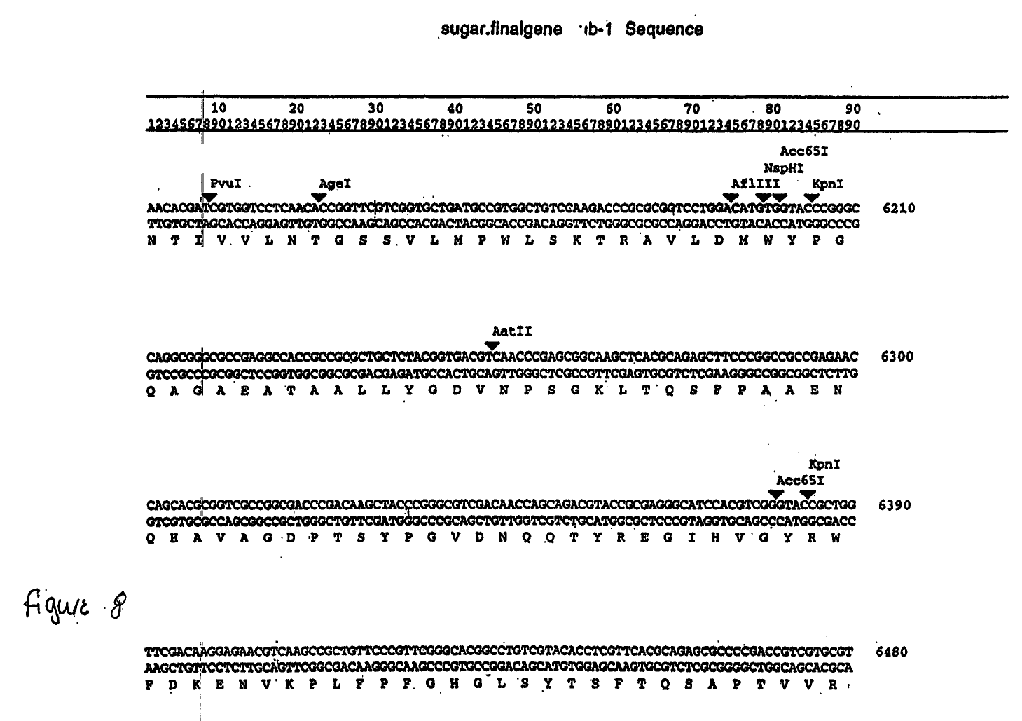

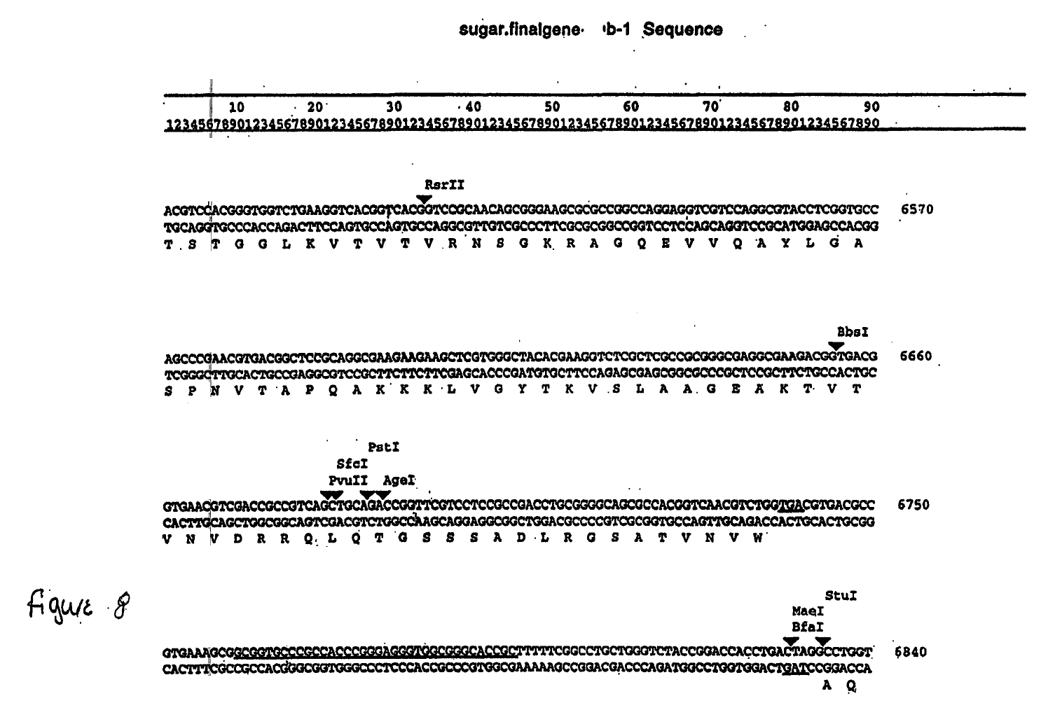

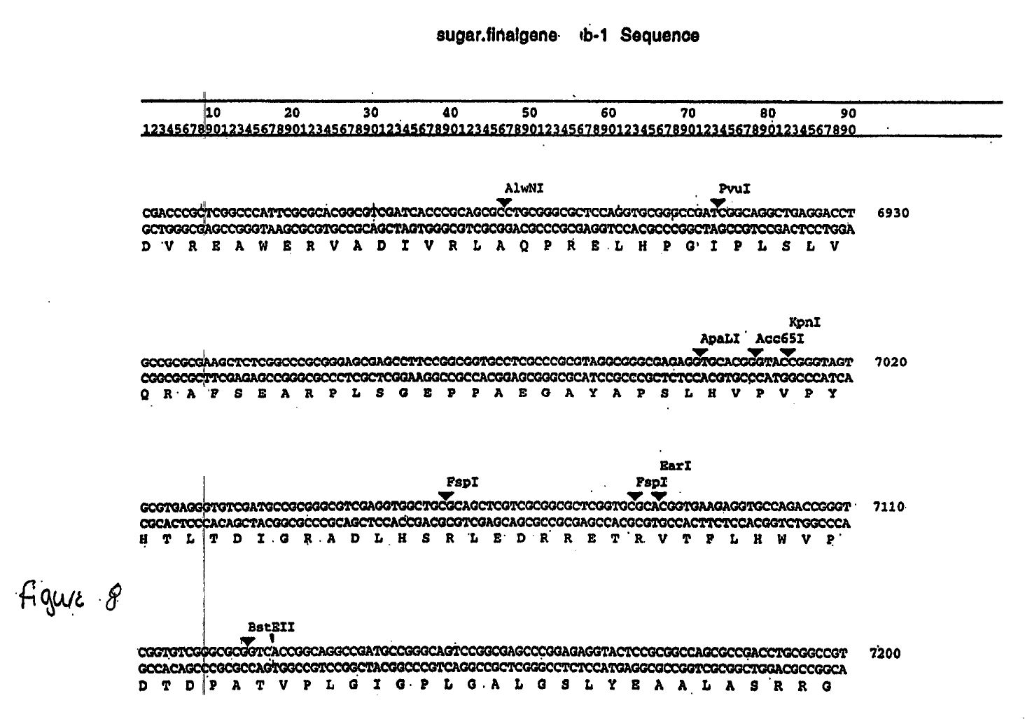

The nucleic acid sequences and segments employed in the invention include those that hybridize under low, moderate or stringent hybridization conditions to the genes encoding sugar biosynthetic enzymes, such as those set forth herein, and/or encode enzymes that have the same or similar activity. A nucleic acid molecule, segment or sequence of the present invention can also be an RNA molecule, segment or sequence which corresponds to, is complementary to or hybridizes under low, moderate, or stringent conditions to any of the DNA segments or sequences described herein. Thus, the invention includes nucleic acid sequences and segments that encode a homolog of a particular sugar biosynthetic enzyme, including a polypeptide that has at least one amino acid substitution (FIG. 9; Alberts et al., 1989), relative to a wild type polypeptide, e.g., the homolog may have at least 29% identity to the wild type polypeptide, as long as the homolog can recognize and catalyze a reaction with a substrate for the wild type enzyme. The homolog may be a naturally occuring enzyme or one that is prepared recombinantly.

Thus, mutations can be made to a native (wild type) nucleic acid segment or sequence of the invention to yield a variant nucleic acid segment or sequence, and such variants may be used in place of the native segment or sequence, so long as the variant encodes an enzyme(s) that functions with other molecules to collectively catalyze the synthesis of an identifiable glycosylatedmolecule such as a glycosylated polyketide or macrolide. Such mutations can be made to the native sequences using conventional techniques such as by preparing synthetic oligonucleotides including the mutations and inserting the mutated sequence into the gene using restriction endonuclease digestion (see, e.g., Kunkel, 1985; Geisselsoder et al., 1987). Alternatively, the mutations can be effected using a mismatched primer (generally 10-20 nucleotides in length) which hybridizes to the native nucleotide segment or sequence, at a temperature below the melting temperature of the mismatched duplex. The primer can be made specific by keeping primer length and base composition within relatively narrow limits and by keeping the mutant base centrally located (Zoller and Smith, 1983). Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. (1982). PCR mutagenesis will also find use for effecting the desired mutations.

Random mutagenesis of the nucleotide sequence can be accomplished by several different techniques known in the art, such as by altering sequences within restriction endonuclease sites, inserting an oligonucleotide linker randomly into a plasmid, by irradiation with X-rays or ultraviolet light, by incorporating incorrect nucleotides during in vitro DNA synthesis, by error-prone PCR mutagenesis, by preparing synthetic mutants or by damaging plasmid DNA in vitro with chemicals. Chemical mutagens include, for example, sodium bisulfite, nitrous acid, hydroxylamine, agents which damage or remove bases thereby preventing normal base-pairing such as hydrazine or formic acid, analogues of nucleotide precursors such as nitrosoguanidine, 5-bromouracil, 2-aminopurine, or acridine intercalating agents such as proflavine, acriflavine, quinacrine, and the like. Generally, plasmid DNA or DNA fragments are treated with chemicals, transformed into E. coli and propagated as a pool or library of mutant plasmids.

Large populations of random enzyme variants can be constructed in vivo using “recombination-enhanced mutagenesis.” This method employs two or more pools of, for example, 10⁶ mutants each of the wild type encoding nucleotide sequence that are generated using any convenient mutagenesis technique and then inserted into cloning vectors.

The gene sequences can be inserted into one or more expression vectors, using methods known to those of skill in the art. Expression vectors may include control sequences operably linked to the desired genes. Suitable expression systems for use with the present invention include systems which function in eukaryotic and prokaryotic host cells. Prokaryotic systems are preferred, and in particular, systems compatible with Streptomyces spp. are of particular interest. Control elements for use in such systems include promoters, optionally containing operator sequences, and ribosome binding sites. Particularly useful promoters include control sequences derived from the gene clusters of the invention. However, other bacterial promoters, such as those derived from sugar metabolizing enzymes, such as galactose, lactose (lac) and maltose, will also find use in the expression cassettes encoding desosamine. Preferred promoters are Streptomyces promoters, including but not limited to the ermE*, pikA and tipA promoters. Additional examples include promoter sequences derived from biosynthetic enzymes such as tryptophan (trp), the β-lactamase (bla) promoter system, bacteriophage lambda PL, and T5. In addition, synthetic promoters, such as the tac promoter (U.S. Pat. No. 4,551,433), which do not occur in nature, also function in bacterial host cells.

Other regulatory sequences may also be desirable which allow for regulation of expression of the genes relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

Selectable markers can also be included in the recombinant expression vectors. A variety of markers are known which are useful in selecting for transformed cell lines and generally comprise a gene whose expression confers a selectable phenotype on transformed cells when the cells are grown in an appropriate selective medium. Such markers include, for example, genes which confer antibiotic resistance or sensitivity to the plasmid.

The various sequences or segments of interest can be cloned into one or more recombinant vectors as individual cassettes, with separate control elements, or under the control of, e.g., a single promoter. The sequences or segments can include flanking restriction sites to allow for the easy deletion and insertion of other sequences or segments. The design of such unique restriction sites is known to those of skill in the art and can be accomplished using the techniques described above, such as site-directed mutagenesis and PCR.

For sequences generated by random mutagenesis, the choice of vector depends on the pool of mutant sequences, i.e., donor or recipient, with which they are to be employed. Furthermore, the choice of vector determines the host cell to be employed in subsequent steps of the claimed method. Any transducible cloning vector can be used as a cloning vector for the donor pool of mutants. It is preferred, however, that phagemids, cosmids, or similar cloning vectors be used for cloning the donor pool of mutant encoding nucleotide sequences into the host cell. Phagemids and cosmids, for example, are advantageous vectors due to the ability to insert and stably propagate therein larger fragments of DNA than in M13 phage and λ phage, respectively. Phagemids which will find use in this method generally include hybrids between plasmids and filamentous phage cloning vehicles. Cosmids which will find use in this method generally include λ phage-based vectors into which cos sites have been inserted. Recipient pool cloning vectors can be any suitable plasmid. The cloning vectors into which pools of mutants are inserted may be identical or may be constructed to harbor and express different genetic markers (see, e.g., Sambrook et al., supra). The utility of employing such vectors having different marker genes may be exploited to facilitate a determination of successful transduction.

Thus, for example, the cloning vector employed may be an E. coli/Streptomyces shuttle vector (see, for example, U.S. Pat. Nos. 4,416,994, 4,343,906, 4,477,571, 4,362,816, and 4,340,674), a cosmid, a plasmid, an artificial bacterial chromosome (see, e.g., Zhang and Wing, 1997; Schalkwyk et al., 1995; and Monaco and Lavin, 1994), or a phagemid, and the host cell may be a bacterial cell such as E. coli, Penicillium patulum, and Streptomyces spp. such as S. lividans, S. venezuelae, or S. lavendulae, or a eukaryotic cell such as fungi, yeast or a plant cell, e.g., monocot and dicot cells, preferably cells that are regenerable.

Moreover, recombinant polypeptides having a particular activity may be prepared via “gene-shuffling”. See, for example, Crameri et al., 1998; Patten et al., 1997, U.S. Pat. Nos. 5,837,458, 5,834,252, 5,830,727, 5,811,238, 5,605,793.

For phagemids, upon infection of the host cell which contains a phagemid, single-stranded phagemid DNA is produced, packaged and extruded from the cell in the form of a transducing phage in a manner similar to other phage vectors. Thus, clonal amplification of mutant encoding nucleotide sequences carried by phagemids is accomplished by propagating the phagemids in a suitable host cell.

Following clonal amplification, the cloned donor pool of mutants is infected with a helper phage to obtain a mixture of phage particles containing either the helper phage genome or phagemids mutant alleles of the wild-type encoding nucleotide sequence.

Infection, or transfection, of host cells with helper phage is generally accomplished by methods well known in the art (see., e.g., Sambrook et al., supra; and Russell et al., 1986).

The helper phage may be any phage which can be used in combination with the cloning phage to produce an infective transducing phage. For example, if the cloning vector is a cosmid, the helper phage will necessarily be a λ phage. Preferably, the cloning vector is a phagemid and the helper phage is a filamentous phage, and preferably phage M13.

If desired after infecting the phagemid with helper phage and obtaining a mixture of phage particles, the transducing phage can be separated from helper phage based on size difference (Barnes et al., 1983), or other similarly effective technique.

The entire spectrum of cloned donor mutations can now be transduced into clonally amplified recipient cells into which has been transduced or transformed a pool of mutant encoding nucleotide sequences. Recipient cells which may be employed in the method disclosed and claimed herein may be, for example, E. coli, or other bacterial expression systems which are not recombination deficient. A recombination deficient cell is a cell in which recombinatorial events is greatly reduced, such as rec⁻ mutants of E. coli (see, Clark et al., 1965).

These transductants can now be selected for the desired expressed protein property or characteristic and, if necessary or desirable, amplified. Optionally, if the phagemids into which each pool of mutants is cloned are constructed to express different genetic markers, as described above, transductants may be selected by way of their expression of both donor and recipient plasmid markers.

The recombinants generated by the above-described methods can then be subjected to selection or screening by any appropriate method, for example, enzymatic or other biological activity.

The above cycle of amplification, infection, transduction, and recombination may be repeated any number of times using additional donor pools cloned on phagemids. As above, the phagemids into which each pool of mutants is cloned may be constructed to express a different marker gene. Each cycle could increase the number of distinct mutants by up to a factor of 10⁶. Thus, if the probability of occurrence of an inter-allelic recombination event in any individual cell is f (a parameter that is actually a function of the distance between the recombining mutations), the transduced culture from two pools of 10⁶ allelic mutants will express up to 10¹² distinct mutants in a population of 10¹²/f cells.

The invention will be further described by the following non-limiting examples.

### EXAMPLE 1

### Deletion of the desR Gene of the Desosamine Biosynthetic Gene Cluster

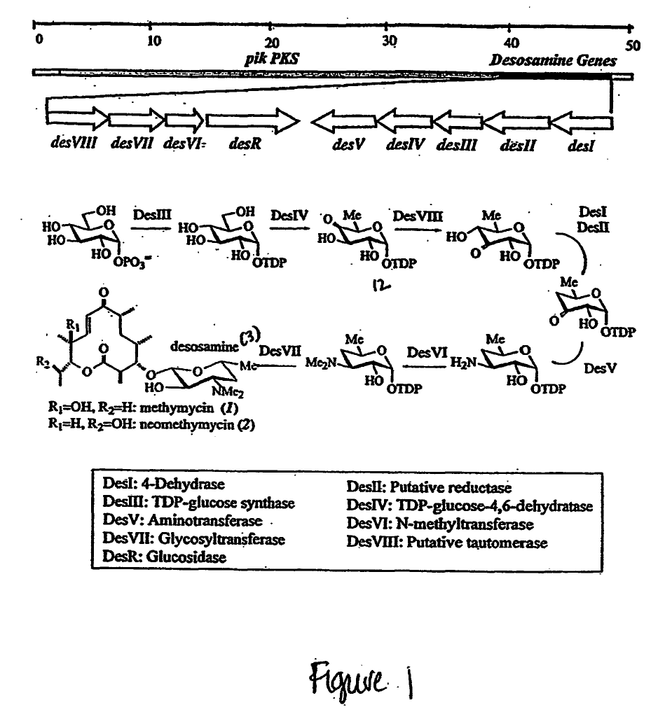

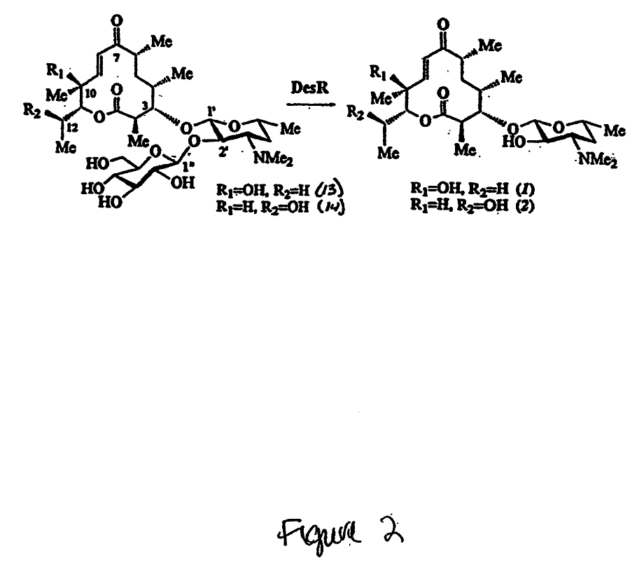

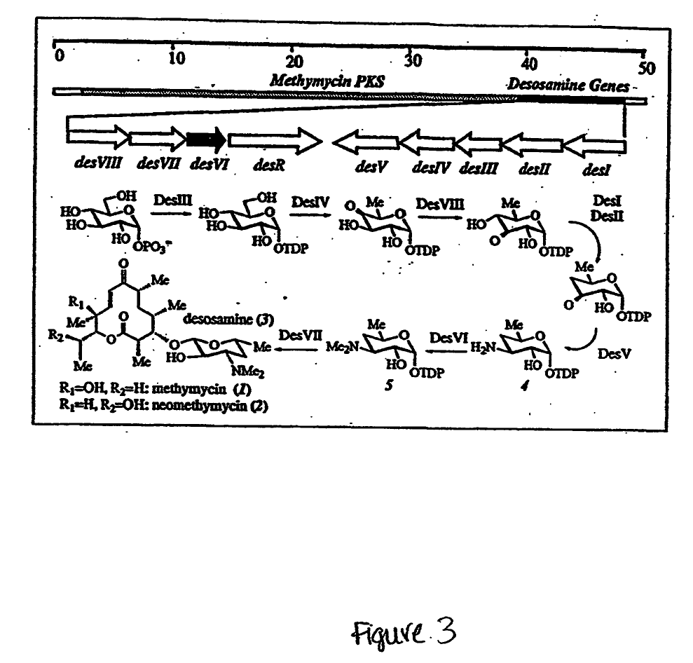

As some macrolides have more than one attached sugar moiety, the assignment of sugar biosynthetic genes to the appropriate sugar biosynthetic pathway can be quite difficult. Since methymycin (a compound of formula (1)) and neomethymycin (a compound of formula (2)) (FIG. 1) (Donin et al., 1953; Djerassi et al., 1956), two closely related macrolide antibiotics produced by Streptomyces venezuelae, contain desosamine as their sole sugar component, the organization of the sugar biosynthetic genes in the methymycin/neomethymycin gene cluster may be less complicated. Thus, this system was chosen for the study of the biosynthesis of desosamine, a N,N-dimethylamino-3,4,6-trideoxyhexose, which also exists in the erythromycin structure (Flinn et al., 1954).

To study the formation of this unusual sugar, a DNA library was constructed by partially digesting the genomic DNA of S. venezuelae (ATCC 15439) with Sau3A I into 35-40 kb fragments which were ligated into the cosmid vector pNJ1 (Tuan et al., 1990). The recombinant DNA was packaged into bacteriophage λ which was used to transfect E. coli DH5α. The resulting cosmid library was screened for desired clones using the tylA1 and tylA2 genes from the tylosin biosynthetic cluster as probes (Baltz et al., 1988; Merson-Davies et al., 1994). These two probes are specific for sugar biosynthetic genes whose products catalyze the first two steps universally followed by all unusual 6-deoxyhexoses studied thus far. The initial reaction involves conversion of glucose-1-phosphate to TDP-D-glucose by α-D-glucose-1-phosphate thymidylyltransferase (TylA1) and subsequently, TDP-D-glucose is transformed to TDP-4-keto-6-deoxy-D-glucose by TDP-D-glucose 4,6-dehydratase (TylA2). Three cosmids were found to contain genes homologous to tylA1 and tylA2. Further analysis of these cosmids led to the identification of nine open reading frames (ORFs) downstream of the PKS genes (FIG. 1). Based on sequence similarities to other sugar biosynthetic genes, especially those derived form the erythromycin cluster (Gaisser et al., 1997; Summers et al., 1997), eight of these nine ORFs are believed to be involved in the biosynthesis of TDP-D-desosamine. Interestingly, the ery cluster lacks homologs of the tylA1 and tylA2 genes that are responsible for the first two steps in desosamine pathway. It is possible that the erythromycin biosynthetic machinery may rely on a general cellular pool of TDP-4-keto-6-deoxy-D-glucose for mycarose and desosamine formation. Depicted in FIG. 1 is a biosynthetic pathway for TDP-D-desosamine.

Although eight of the nine ORFs have been assigned to desosamine formation, the presence of desR, which shows strong sequence homology to β-glucosidases (as high as 39% identity and 46% similarity) (Castle et al., 1998), within the desosamine gene cluster is puzzling. To investigate the function of DesR relative to the biosynthesis of methymycin/neomethymycin, a disruption plasmid (pBL1005) derived from pKC1139 (containing an apramycin resistance marker) (Bierman et al., 1992) was constructed in which a 1.0 kb NcoI/XhoI fragment of the desR gene was deleted and replaced by the thiostrepton resistance (tsr) gene (1.1 kb) (Bibb et al., 1985) via blunt-end ligation. This plasmid was used to transform E. coli S17-1; which serves as the donor strain to introduce the pBL1005 construct through conjugal transfer into the wild-type S. venezuelae (Bierman et al., 1992). The double crossover mutants in which chromosomal desR had been replaced with the disrupted gene were selected according to their thiostrepton-resistant and apramycin-sensitive characteristics. Southern blot hybridization analysis was used to confirm the gene replacement.

The desired mutant was first grown at 29° C. in seed medium for 48 hours, and then inoculated and grown in vegetative medium for another 48 hours (Cane et al., 1993). After the fermentation broth was centrifuged at 10,000 g to remove cellular debris and mycelia, the supernatant was adjusted to pH 9.5 with concentrated KOH, and extracted with an equivolume of chloroform (four times). The organic layer was dried over sodium sulfate and evaporated to dryness. The amber oil-like crude products were first subjected to flash chromatography on silica gel using a gradient of 0-40% methanol in chloroform, followed by HPLC purification on a C₁₈ column eluted isocratically with 45% acetonitrile in 57 mM ammonium acetate (pH 6.7). In addition to methymycin (a compound of formula (1)) and neomethymycin (a compound of formula (2)), two new products were isolated. The yield of a compound of formula (13) and a compound of formula (14) was each in the range of 5-10 mg/L of fermentation broth. However, a compound of formula (1) and a compound of formula (2) remained to be the major products. High-resolution FAB-MS revealed that both compounds have identical molecular compositions that differ from methymycin/neomethymycin by an extra hexose. The chemical nature of these two new compounds were elucidated to be C-2′β-glucosylated methymycin and neomethymycin (a compound of formula (13) and formula (14), respectively) by extensive spectral analysis.

The spectral data of (13): ¹H NMR (acetone-d₆) δ 6.56 (1H, d, J=16.0, 9-H), 6.46 (1H, d, J=16.0, 8-H), 4.67 (1H, dd, J=10.8, 2.0, 11-H), 4.39 (1H, d, J=7.5, 1′-H), 4.32 (1H, d, J=8.0, 1″-H), 3.99 (1H, dd, J=11.5, 2.5, 6″-H), 3.72 (1H, dd, J=11.5, 5.5, 6″-H), 3.56 (1H, m, 5′-H), 3.52 (1H, d, J=10.0, 3-H), 3.37 (1H, t, J=8.5, 3″-H), 3.33 (1H, m, 5″-H), 3.28 (1H, t, J=8.5, 4″-H), 3.23 (1H, dd, J=10.5, 7.5, 2′-H), 3.15 (1H, dd, J=8.5, 8.0, 2″-H), 3.10 (1H, m, 2-H), 2.75 (1H, 3′-H, buried under H₂O peak), 2.42 (1H, m, 6-H), 2.28 (6H, s, NMe₂), 1.95 (1H, m, 12-H), 1.9 (1H, m, 5-H), 1.82 (1H, m, 4′-H), 1.50 (1H, m, 12-H), 1.44 (3H, d, J=7.0, 2-Me), 1.4 (1H, m, 5-H), 1.34 (3H, s, 10-Me), 1.3 (1H, m, 4-H), 1.25 (1H, m, 4′-H), 1.20 (3H, d, J=6.0, 5′-Me), 1.15 (3H, d, J=7.0, 6-Me), 0.95 (3H, d, J=6.0, 4-Me), 0.86 (3H, t, J=7.5, 12-Me). High-resolution FAB-MS: calc for C₃₁H₅₄NO₁₂ (M+H)⁺ 632.3646, found 632.3686.

Spectral data of (14): ¹H NMR (acetone-d₆) δ 6.69 (1H, dd, J=16.0, 5.5 Hz, 9-H), 6.55 (1H, dd, J=16.0, 1.3, 8-H), 4.71 (1H, dd, J=9.0, 2.0, 11-H), 4.37 (1H, d, J=7.0, 1′-H), 4.31 (1H, d, J=8.0, 1″-H), 3.97 (1H, dd, J=11.5, 2.5, 6″-H), 3.81 (1H, dq, J=9.0, 6.0, 12-H), 3.72 (1H, dd, J=11.5, 5.0, 6″-H), 3.56 (1H, m, 5′-H), 3.50 (1H, bd, J=10.0, 3-H), 3.36 (1H, t, J=8.5, 3″-H), 3.32 (1H, m, 5″-H), 3.30 (1H, t, J=8.5, 4″-H), 3.23 (1H, dd, J=10.2, 7.0, 2′-H), 3.13, (1H, dd, J=8.5, 8.0, 2″-H), 3.09 (1H, m, 2-H), 3.08 (1H, m, 10-H), 2.77 (1H, ddd, J=12.5, 10.2, 4.5, 3′-H), 2.41 (1H, m, 6-H), 2.28 (6H, s, NMe₂), 1.89 (1H, t, J=13.0, 5-H), 1.83 (1H, ddd, J=12.5, 4.5, 1.5, 4′-H), 1.41 (3H, d, J=7.0, 2-Me), 1.3 (1H, m, 4-H), 1.25 (1H, m, 5-H), 1.2 (1H, m, 4′-H, 1.20 (3H, d, J=6.0, 5′-Me), 1.17 (6H, d, J=7.0, 6-Me, 10-Me), 1.12 (3H, d, J=6.0, 12-me), 0.96 (3H, d, J=6.0, 4-Me). ¹³C NMR (acetone-d₆) δ 204.1 (C-7), 175.8 (C-1), 148.2 (C-9), 126.7 (C-8), 108.3 (C-1″), 104.2 (C-1′), 85.1 (C-3), 83.0 (C-2′), 78.2 (C-3″), 78.1 (C-5″), 76.6 (C-2″), 76.4 (C-11), 71.8 (C-4″), 69.3 (C-5′), 66.1 (C-12), 66.0 (C-3′), 63.7 (C-6″), 46.2 (C-6), 44.4 (C-2), 40.8 (NMe₂), 36.4 (C-10), 34.7 (C-5), 34.0 (C-4′), 29.5 (C-4′), 21.5 (5′-Me), 21.5 (12-Me), 17.9 (6-Me), 17.7 (4-Me), 17.2 (2-Me), 9.9 (10-Me). High-resolution FAB-MS: calc for C₃₁H₅₄NO₁₂ (M+H)⁺ 632.3646, found 632.3648.

The coupling constant (d, J=8.0 Hz) of the anomeric hydrogen (1″-H) of the added glucose and the magnitude of the downfield shift (11.8 ppm) of C-2′ of desosamine are all consistent with the assigned C-2′β-configuration (Seo et al., 1978).

The antibiotic activity of a compound of formula (13) and (14) against Streptococcus pyogenes was examined by separately applying 20 μL of each sample (1.6 mM in MeOH) to sterilized filter paper discs which were placed onto the surface of S. pyogenes grown on Mueller-Hinton agar plates (Mangahas, 1996). After being grown overnight at 37° C., the plates of the controls (a compound of formula (1) and (2)) showed clearly visible inhibition zones. In contrast, no such clearings were discernible around the discs of a compound of formula (13) and (14). Evidently, β-glucosylation at C-2′ of desosamine in methymycin/neomethymycin renders these antibiotics inactive.

It should be noted that similar phenomena involving inactivation of macrolide antibiotics by glycosylation are known (Celmer et al., 1985; Kuo et al., 1989; Sasaki et al., 1996). For example, it was found that when erythromycin was given to Streptomyces lividans, which contains a macrolide glycosyltransferase (MgtA), the bacterium was able to defend itself by glycosylating the drug (Cundliffe, 1992; Jenkins et al., 1991). Such a macrolide glycosyltransferase activity has been detected in 15 out of a total of 32 actinomycete strains producing various polyketide antibiotics (Sasaki et al., 1996). Interestingly, the co-existence of a macrolide glycosyltransferase (OleD) capable of deactivating oleandomycin by glucosylation (Hernandez et al., 1993), and an extracellular β-glucosidase capable of removing the added glucose from the deactivated oleandomycin in Streptomyces antibioticus (Vilches et al., 1992) has led to the speculation of glycosylation as a possible self-resistance mechanism in S. antibioticus. Although the genes of the aforementioned glycosyltransferases have been cloned in a few cases, such as mgtA of S. lividans and oleD of S. anitibioticus, the whereabouts of macrolide β-glycosidase genes remain obscure. Interestingly, the recently released eryBI sequence, which is part of the erythromycin biosynthetic cluster, is highly homologous to desR (55% identity) (Gaisser et al., 1997).

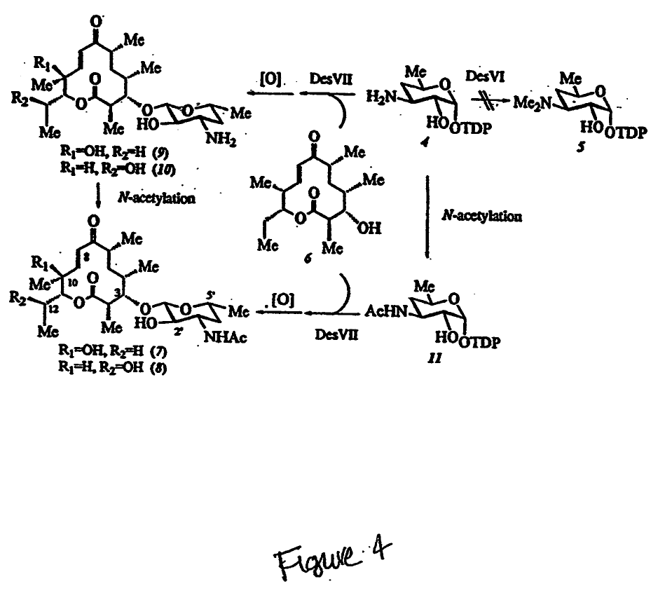

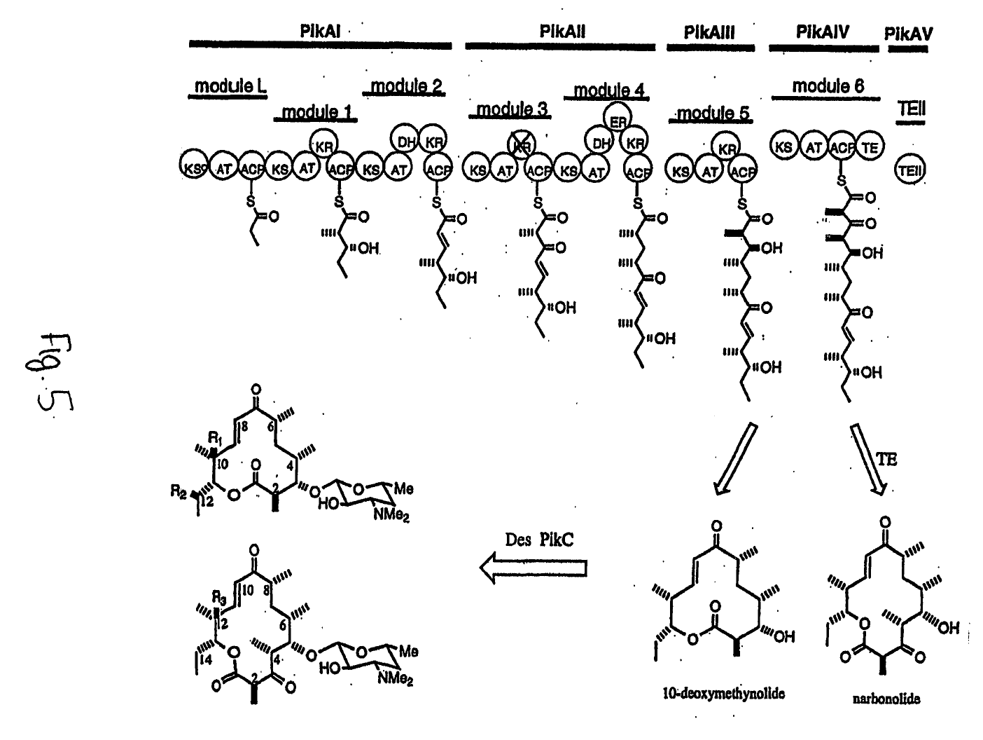

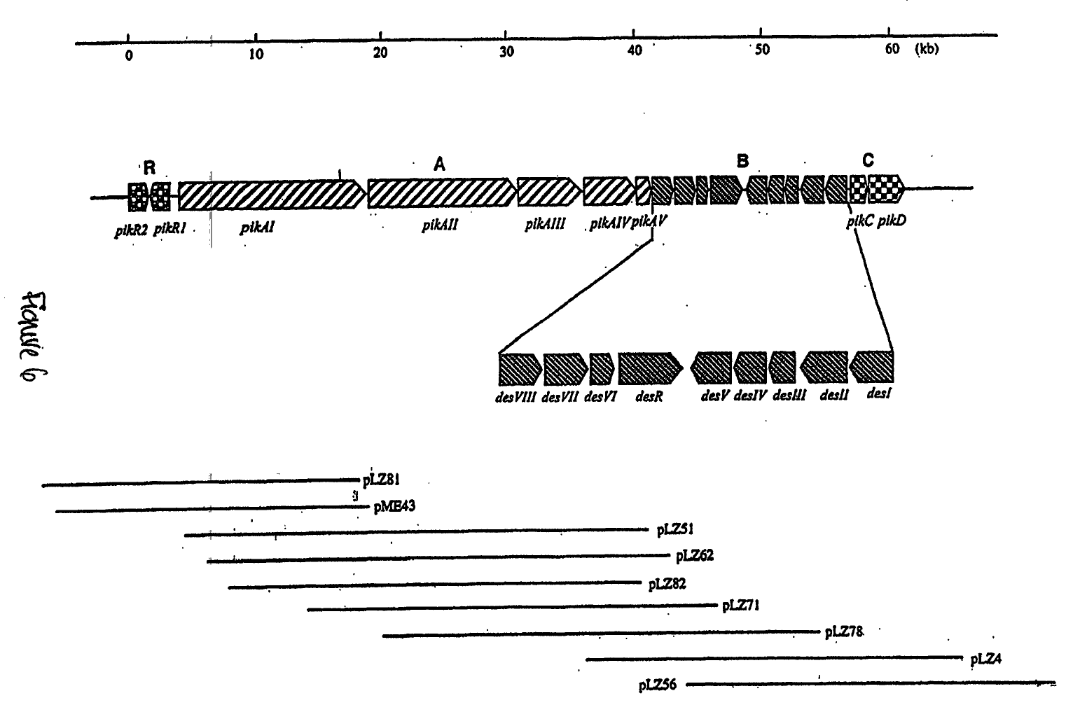

The discovery of desR, a macrolide β-glucosidase gene, within the desosamine gene cluster is thus significant, and the accumulation of deactivated compounds of formula (13) and (14) after desR disruption provides direct molecular evidence indicating that a similar self-defense mechanism via glycosylation/deglycosylation may also be operative in S. venezuelae. However, because a significant amount of methymycin and neomethymycin also exist in the fermentation broth of the mutant strain, glucosylation of desosamine may not be the primary self-resistance mechanism in S. venezuelae. Indeed, an rRNA methyltransferase gene found upstream from the PKS genes in this cluster may confer the primary self-resistance protection. Thus, these results are consistent with the fact that antibiotic producing organisms generally have more than one defensive option (Cundliffe, 1989). In light of this observation, it is conceivable that methymycin/neomethymycin may be produced in part as the inert diglycosides (a compound of formula (13) or (14)), and the macrolide β-glucosidase encoded by desR is responsible for transforming methymycin/neomethymycin from their dormant state to their active form. Supporting this idea, the translated desR gene has a leader sequence characteristic of secretory proteins (von Heijne, 1986; von Heijne, 1989). Thus, DesR may be transported through the cell membrane and hydrolyze the modified antibiotics extracellularly to activate them (FIG. 2).

Summary

Inspired by the complex assembly and the enzymology of aminodeoxy sugars that are frequently found as essential components of macrolide antibiotics, the entire desosamine biosynthetic gene cluster from the methymycin and neomethymycin producing strain Streptomyces venezuelae was cloned, sequenced, and mapped. Eight of the nine mapped genes were assigned to the biosynthesis of TDP-D-desosamine based on sequence similarities to those derived from the erythromycin cluster. The remaining gene, designated desR, showed strong sequence homology to β-glucosidases.

To investigate the function of the encoded protein (DesR), a disruption mutant was constructed in which a NcoI/XhoI fragment of the desR gene was deleted and replaced by the thiostrepton resistance (tsr) gene. In addition to methymycin and neomethymycin, two new products were isolated from the fermentation of the mutant strain. These two new compounds, which are biologically inactive, were found to be C-2′β-glucosylated methymycin and neomethymycin. Since the translated desR gene has a leader sequence characteristic of secretory proteins, the DesR protein may be an extracellular β-glucosidase capable of removing the added glucose from the modified antibiotics to activate them. Thus, the occurrence of desR within the desosamine gene cluster and the accumulation of deactivated glucosylated methymycin/neomethymycin upon disruption of desR provide strong molecular evidence suggesting that a self-resistance mechanism via glucosylation may be operative in S. venezuelae.

Thus, the desR gene can be used as a probe to identify homologs in other antibiotic biosynthetic pathways. Deletion of the corresponding macrolide glycosidase gene in other antibiotic biosynthetic pathways may lead to the accumulation of the glycosylated products which may be used as prodrugs with reduced cytotoxicity. Glycosylation also holds promise as a tool to regulate and/or minimize the potential toxicity associated with new macrolide antibiotics produced by genetically engineered microorganisms. Moreover, the availability of macrolide glycosidases, which can be used for the activation of newly formed antibiotics that have been deliberately deactivated by engineered glycosyltransferases, may be useful in the development of novel antibiotics using the combinatorial biosynthetic approach (Hopwood et al., 1990; Katz et al., 1993; Hutchinson et al., 1995; Carreras et al., 1997; Kramer et al., 1996; Khosla 25 et al., 1996; Jacobsen et al., 1997; Marsden et al., 1998).

### EXAMPLE 2

### Deletion of the DesVI Gene of the Desosamine Biosynthetic Gene Cluster

The emergence of pathogenic bacteria resistant to many commonly used antibiotics poses a serious threat to human health and has been the impetus of the present resurgent search for new antimicrobial agents (Box et al., 1997; Davies, 1996; Service, 1995). Since the first report on using genetic engineering techniques to create “hybrid” polyketides (Hopwood et al., 1995), the potential of manipulating the genes governing the biosynthesis of secondary metabolites to create new bioactive compounds, especially macrolide antibiotics, has received much attention (Kramer et al., 1996; Khosla et al., 1996). This class of clinically important drugs consists of two essential structural components: a polyketide aglycone and the appended deoxy sugars (Omura, 1984). The aglycone is synthesized via sequential condensations of acyl thioesters catalyzed by a highly organized multi-enzyme complex, polyketide synthase (PKS) (Hopwood et al., 1990; Katz, 1993; Hutchinson et al., 1995; Carreras et al., 1997). Recent advances in the understanding of the polyketide biosynthesis have allowed recombination of the PKS genes to construct an impressive array of novel skeletons (Kramer et al., 1996; Khosla et al., 1996; Hopwood et al., 1990; Katz, 1993; Hutchinson et al., 1995; Carreras et al., 1997; Epp et al., 1989; Donadio et al., 1993; Arisawa et al., 1994; Jacobsen et al., 1997; Marsden et al., 1998). Without the sugar components, however, these new compounds are usually biologically impotent. Hence, if one plans to make new macrolide antibiotics by a combinatorial biosynthetic approach, two immediate challenges must be overcome: assembling a repertoire of novel sugar structures and then having the capacity to couple these sugars to the structurally diverse macrolide aglycones.

Unfortunately, knowledge of the formation of the unusual sugars in these antibiotics remains limited (Liu et al., 1994; Kirschning et al., 1997; Johnson et al., 1998). Part of the reason for this comes from the fact that the sugar genes are generally scattered at both ends of the PKS genes. Such an organization within the macrolide biosynthetic gene cluster makes it difficult to distinguish the sugar genes from those encoding regulatory proteins or aglycone modification enzymes that are also interspersed in the same regions. The task can be made even more formidable if the macrolides contain multiple sugar components. In view of the “scattered” nature of the sugar biosynthetic genes, the antibiotic methymycin (a compound of formula (1) in FIG. 1) and its co-metabolite, neomethymycin (a compound of formula (2) in FIG. 1)), of Streptomyces venezuelae present themselves as an attractive system to study the formation of deoxy sugars (Donin et al., 1953; Djerassi et al., 1956). First, they carry D-desosamine (a compound of formula (3)) a prototypical aminodeoxy sugar that also exists in erythromycin. Second, since desosamine is the only sugar attached to the macrolactone of formula (1) and (2), identification of the sugar biosynthetic genes within the methymycin/neomethymycin gene cluster should be possible with much more certainty.

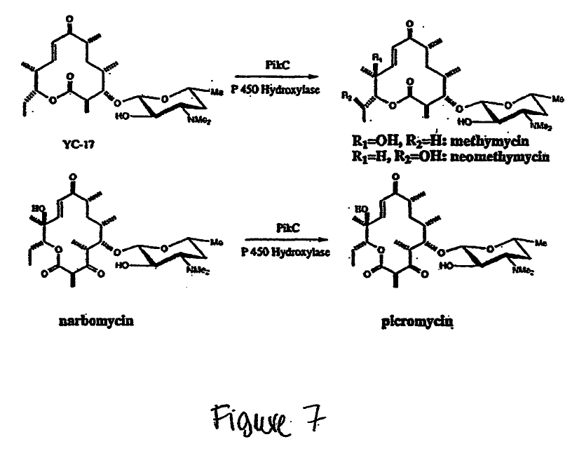

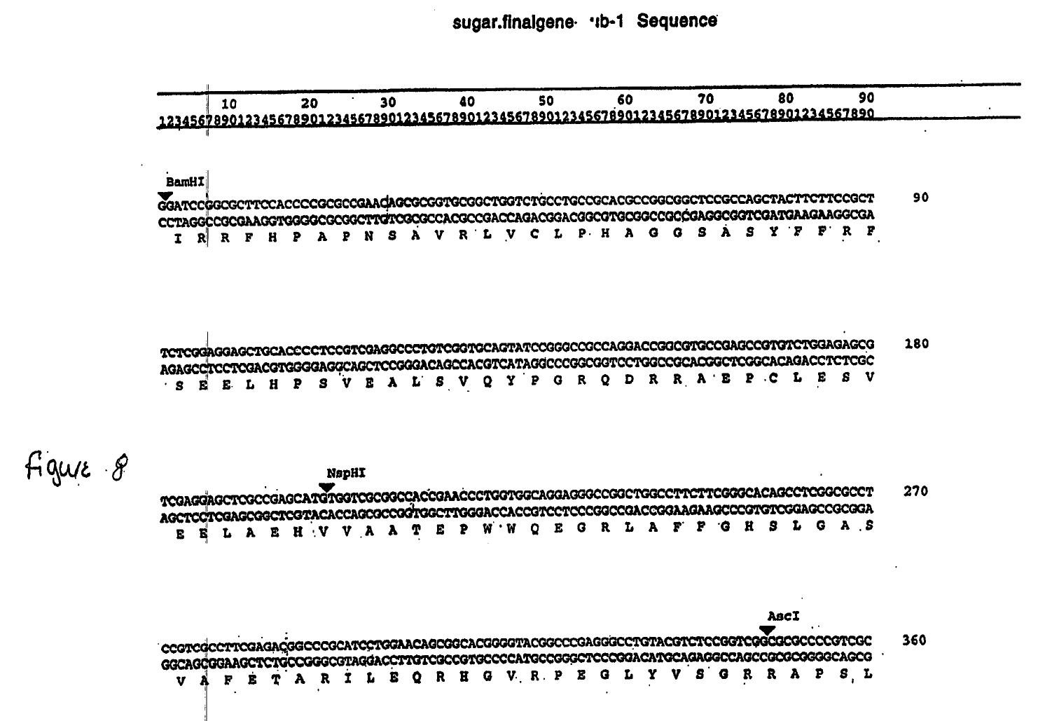

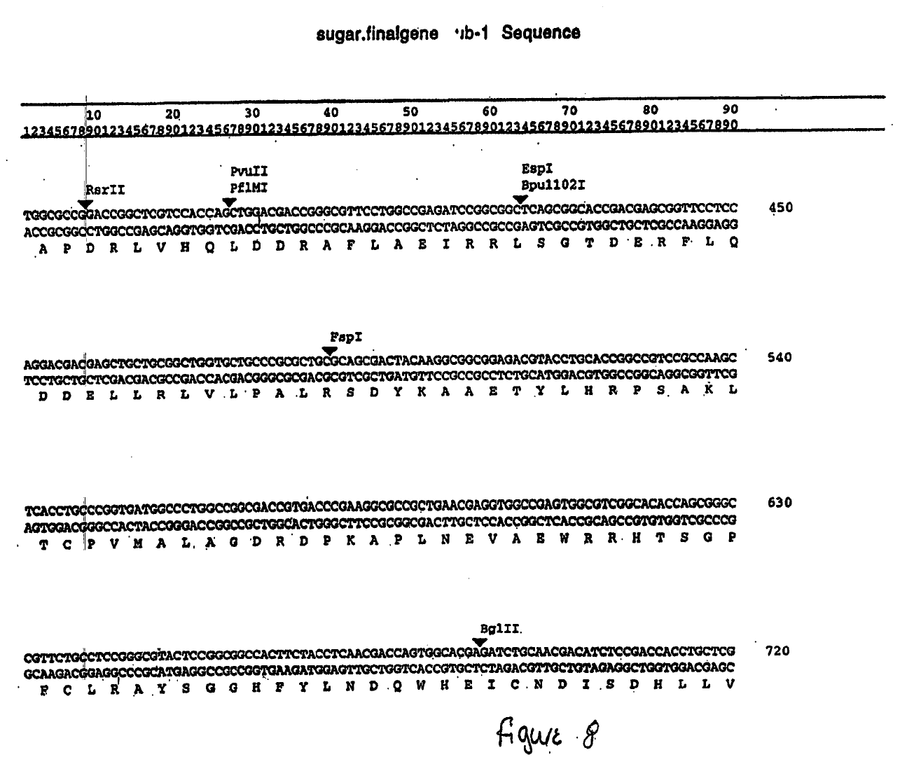

A 10 kb stretch of DNA downstream from the methymycin/neomethymycin gene cluster, which is about 60 kb in length, was found to harbor the entire desosamine biosynthetic gene cluster (FIG. 3). Among the nine open reading frames (ORFs) mapped in this segment, eight are likely to be involved in desosamine formation, while the remaining one, desR, encodes a macrolide β-glycosidase that may be involved in a self-resistance mechanism. Their identities, shown in FIG. 3, are assigned based on sequence similarities to other sugar biosynthetic genes (Gaisser et al., 1997; Summers et al., 1997). The proposed pathway is well founded on literature precedent and mechanistic intuition for the construction of aminodeoxy sugars (Liu et al., 1994; Kirschning et al., 1997; Johnson et al., 1998).

To determine whether new methymycin/neomethymycin analogues carrying modified sugars could be generated by altering the desosamine biosynthetic genes, the desVI gene, which has been predicted to encode the N-methyltransferase, was chosen as a target (Gaisser et al., 1997; Summers et al., 1997). The deduced desVI product is most closely related to that of eryCVI from the erythromycin producing strain Saccharopolyspora erythraea (70% identity), and also strongly resembles the predicted products of rdmD from the rhodomiycin cluster of Streptomyces purpurascens (Niemi et al., 1995), srmX from the spiromycin cluster of Streptomyces ambofaciens (Geistlich et al., 1992), and tylM1 from the tylosin cluster of Streptomyces fradiae (Gandecha et al., 1997). All of these enzymes contain the consensus sequence LLDV(I)ACGTG (SEQ ID NO:25) (Gaisser et al., 1997; Summers et al., 1997), near their N-terminus, which is part of the S-adenosylmethionine binding site (Ingrosso et al., 1989; Haydock et al., 1991).

The deletion of desVI should have little polar effect (Lin et al., 1984) on the expression of other desosamine biosynthetic genes because the ORF (desR) lying immediately downstream from desVI is not directly involved in desosamine formation, and those lying further downstream are transcribed in the opposite direction. Second, since N,N-dimethylation is almost certainly the last step in the desosamine biosynthetic pathway (Liu et al., 1994; Kirschning et al., 1997; Johnson et al., 1998; Gaisser et al., 1997; Summers et al., 1997), perturbing this step may lead to the accumulation of a compound of formula (4), which stands the best chance among all other intermediates of being recognized by the glycosyltransferase (DesVII) for successful linkage to the macrolactone of formula (6) (FIG. 2). Deletion and/or disruption of a single biosynthetic gene often affects the pathway at more than one specific step. In fact, disruption of eryCVI, the desVI equivalent in the erythromycin cluster, which has been predicted to encode a similar N-methylase to make desosamine in erythromycin (Gaisser et al., 1997; Summers et al., 1997), led to the accumulation of an intermediate devoid of the entire desosamine moiety (Summers et al., 1997).

A plasmid pBL3001, in which desVI was replaced by the thiostrepton gene (tsr) (Bibb et al., 1985), was constructed and introduced into wild type S. venezuelae by conjugal transfer using E. coli S17-1 (Bierman et al., 1992). Two identical double crossover mutants, KdesVI-21 and KdesVI-22 with phenotypes of thiostrepton resistance (Thio^{R}) and apamycin sensitivity (Apm^{S}) were obtained. Southern blot hybridization using tsr or a 1.1 kb HincII fragment from the desVII region further confirmed that the desVI gene was indeed replaced by tsr on the chromosome of these mutants. The KdesVI-21 mutant was first grown at 29° C. in seed medium (100 mL) for 48 hours, and then inoculated and grown in vegetative medium (3 L) for another 48 hours (Cane et al., 1993). The fermentation broth was centrifuged to remove the cellular debris and mycelia, and the supernatant was adjusted to pH 9.5 with concentrated KOH, followed by extraction with chloroform. No methymycin or neomethymycin was found; instead, the 10-deoxy-methynolide (6) (350 mg) (Lambalot et al., 1992) and two new macrolides containing an N-acetylated amino sugar, a compound of formula (7) (20 mg) and a compound of formula (8) (15 mg), were isolated. Their structures were determined by spectral analyses and high-resolution MS.

Spectral data of formula 7 are: ¹H NMR (CDCl₃) δ 6.62 (1H, d, J=16.0, H-9), 6.22 (1H, d, J=16.0, H-8), 5.75 (1H, d, J=7.5, N—H), 4.75 (1H, dd, J=10.8, 2.2, H-11), 4.28 (1H, d, J=7.5, H-1′), 3.95 (1H, m, H-3′), 3.64 (1H, d, J=10.5, H-3), 3.56 (1H, m, H-5′), 3.16 (1H, dd, J=10.0, 7.5, H-2′), 2.84 (1H, dq, J=10.5, 7.0, H-2), 2.55 (1H, m, H-6), 2.02 (3H, s, NAc), 1.95 (1H, m, H-12), 1.90 (1H, m, H-4′), 1.66 (1H, m, H-5), 1.50 (1H, m, H-12), 1.41 (3H, d, J=7.0, 2-Me), 1.40 (1H, m, H-5), 1.34 (3H, s, 10-Me), 1.25 (1H, m, H-4), 1.22 (1H, m, H-4′), 1.21 (3H, d, J=6.0, H-6′), 1.17 (3H, d, J=7.0, 6-Me), 1.01 (3H, d, J=6.5, 4-Me), 0.89 (3H, t, J=7.2, 12-Me); ¹³C NMR (CDCl₃) δ 204.3 (C-7), 175.1 (C-1), 171.8 (Me—C═O), 149.1 (C-9), 125.3 (C-8), 104.4 (C-1′), 85.4 (C-3), 76.3 (C-11), 75.4 (C-2′), 74.1 (C-10), 68.6 (C-5′), 51.9 (C-3′), 45.0 (C-6), 44.0 (C-2), 38.5 (C-4′), 33.8 (C-5), 33.3 (C-4), 23.1 (Me—C═O), 21.1 (C-12), 20.6 (C-6′), 19.2 (10-Me), 17.5 (6-Me), 17.2 (4-Me), 16.2 (2-Me), 10.6 (12-Me). High-resolution FABMS: calc for C₂₅H₄₃O₈N (M+H)⁺ 484.2910, found 484.2903.

Spectral data of formula 8 are: ¹H NMR (CDCl₃) δ 6.76 (1H, dd, J=16.0, 5.5, H-9), 6.44 (1H, dd, J=16.0, 1.5, H-8), 5.50 (1H, d, J=6.5, N—H), 4.80 (1H, dd, J=9.0, 2.0, H-11), 4.28 (1H, d, J=7.5, H-1′), 3.95 (1H, m, H-3′), 3.88 (1H, m, H-12), 3.62 (1H, d, J=11.0, H-3), 3.57 (1H, m, H-5′), 3.18 (1H, dd, J=10.0, 7.5, H-2′), 3.06 (1H, m, H-10), 2.86 (1H, dq, J=11.0, 7.0, H-2), 2.54 (1H, m, H-6), 2.04 (3H, s, NAc), 1.98 (1H, m, H-4′), 1.67 (1H, m, H-5), 1.40 (1H, m, H-5), 1.39 (3H, d, J=7.0, 2-Me), 1.25 (1H, m, H-4), 1.22 (1H, m, H-4′), 1.22 (3H, d, J=6.0, H-6′), 1.21 (3H, d, J=6.0, 6-Me), 1.19 (3H, d, J=7.0, 12-Me), 1.16 (3H, d, J=6.5, 10-Me), 1.01 (3H, d, J=6.5, 4-Me); ¹³C NMR (CDCl₃) δ 205.1 (C-7), 174.6 (C-1), 171.9 (Me—C═O), 147.2 (C-9), 126.2 (C-8), 104.4 (C-1′), 85.3 (C-3), 75.7 (C-11), 75.4 (C-2′), 68.7 (C-5′), 66.4 (C-12), 52.0 (C-3′), 45.1 (C-6), 43.8 (C-2), 38.6 (C4′), 35.4 (C-10), 34.1 (C-5), 33.4 (C4), 23.1 (Me—C═O), 21.0 (12-Me), 20.7 (C-6′), 17.7 (6-Me), 17.4 (4-Me), 16.1 (2-Me), 9.8 (10-Me). High-resolution FABMS: calc for C₂₅H₄₃O₈N (M+H)⁺ 484.2910, found 484.2892.

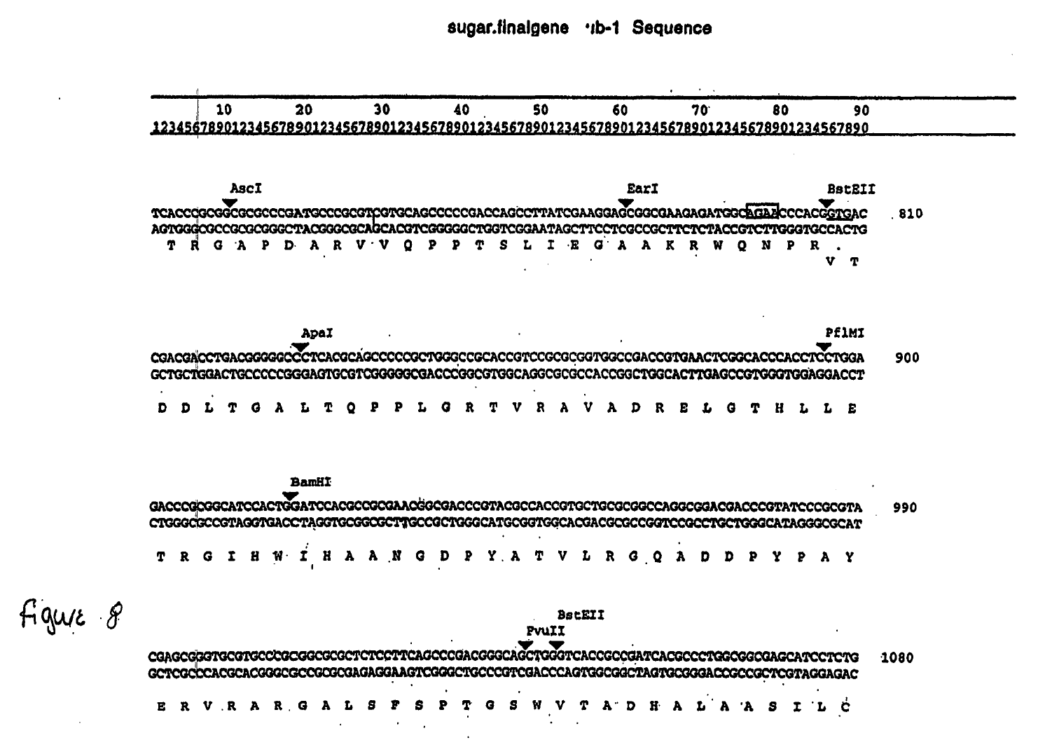

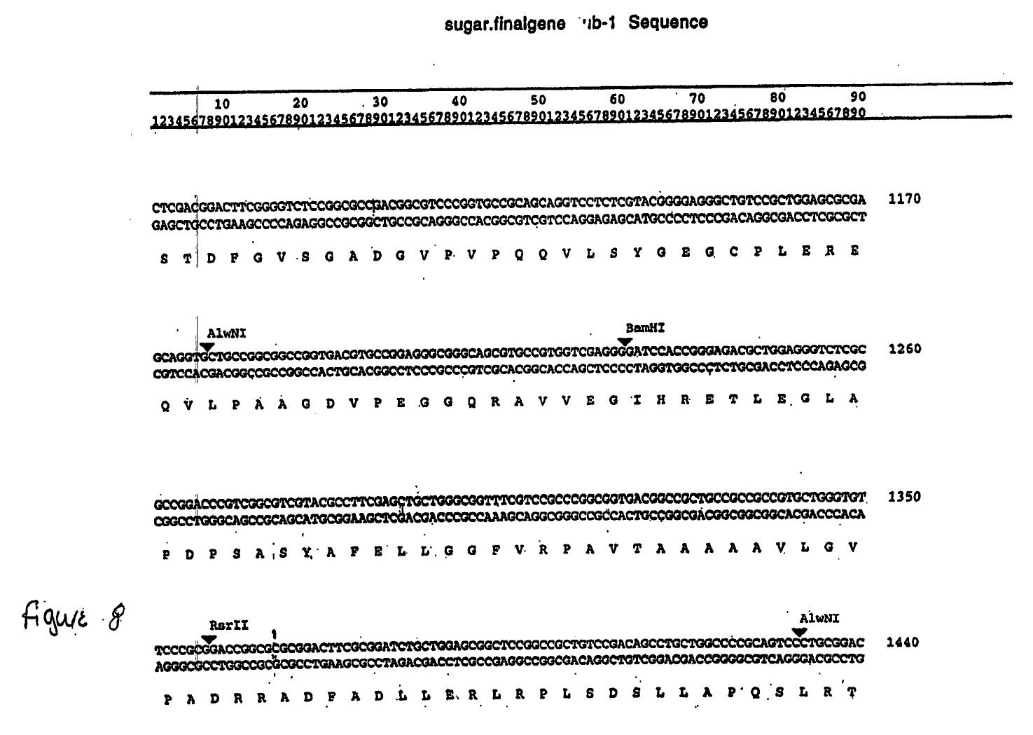

The fact that compounds of formula (7) and (8) bearing modified desosamine are produced by the desVI-deletion mutant is a thrilling discovery. However, this result is also somewhat surprising since the sugar component in the products is expected to be the aminodeoxy hexose (4). As illustrated in FIG. 4, it is possible that a compound of formula (7) and (8) are derived from the predicted compound of formula (9) and (10), respectively, by a post-synthetic nonspecific acetylation of the attached aminodeoxy sugar. It is also conceivable that N-acetylation of (4) occurs first, followed by coupling of the resulting sugar (11) to the 10-deoxymethynolide (6). Nevertheless, the lack of N-methylation of the sugar component in these new products provides convincing evidence sustaining the assignment of desVI as the N-methyltransferase gene. Most significantly, the production of a compound of formula (7) and (8) by the desVI-deletion mutant attests to the fact that the glycosyltransferase (DesVII) in methymycin/neomethymycin pathway is capable of recognizing and processing sugar substrates other than TDP-desosamine (5).

Since both compounds of formula (7) and (8) are new compounds synthesized in vivo by the S. venezuelae mutant strain, the observed N-acetylation might be a necessary step for self-protection (Cundliffe, 1989). In view of these results, the potential toxicity associated with new macrolide antibiotics produced by genetically engineered microorganisms can be minimized and newly formed antibiotics that have been deactivated (either deliberately or not) during production can be activated. Such an approach can be part of an overall strategy for the development of novel antibiotics using the combinatorial biosynthetic approach. Indeed, purified compounds of formula (7) and (8) are inactive against Streptococcus pyogenes grown on Mueller-Hinton agar plates (Mangahas, 1996), while the controls (a compound of formula (1) and (2)) show clearly visible inhibition zones.

It should be pointed out that a few glycosyltransferases involved in the biosynthesis of antibiotics have been shown to have relaxed specificity towards modified macrolactones (Jacobsen et al., 1997; Marsden et al., 1998; Weber et al., 1991). However, a similar relaxed specificity toward sugar substrates has only been reported for the daunorubicin glycosyltransferase, which is able to recognize a modified daunosamine and catalyze its coupling to the aglycone, ε-rhodomycinone (Madduri et al., 1998). Thus, the fact that the methymycin/neomethymycin glycosyltransferase can also tolerate structural variants of its sugar substrate indicates that at least some glycosyltransferases in antibiotic biosynthetic pathways may be useful to create biologically active hybrid natural products via genetic engineering.

Summary

The appended sugars in macrolide antibiotics are indispensable to the biological activities of these clinically important drugs. Therefore, the development of new antibiotics via a biological combinatorial approach requires detailed knowledge of the biosynthesis of these unusual sugars, as well as the ability to manipulate the biosynthetic genes to create novel sugars that can be incorporated into the final macrolide structures. A targeted deletion of the desVI gene of Streptomyces venezuelae, which has been predicted to encode an N-methyltransferase based on sequence comparison, was prepared to determine whether new methymycin/neomethymycin analogues bearing modified sugars can be generated by altering the desosamine biosynthetic genes. Growth of the S. venezuelae deletion mutant strain resulted in the accumulation of a methymycin/neomethymycin analogue carrying an N-acetylated aminodeoxy sugar. Isolation and characterization of these derivatives not only provide the first direct evidence confirming the identity of desVI as the N-methyltransferase gene, but also demonstrate the feasibility of preparing novel sugars by the gene deletion approach. Most significantly, the results also revealed that the glycosyltransferase of methymycin/neomethymycin exhibits a relaxed specificity towards its sugar substrates.

### EXAMPLE 3

### Cloning and Sequencing of the Met/Pik Biosynthetic Gene Cluster

Materials and Methods

Bacterial Strains and Media. E. coli DH5α was used as a cloning host. E. coli LE392 was the host for a cosmid library derived from S. venezuelae genomic DNA. LB medium was used in E. coli propagation. Streptomyces venezuelae ATCC 15439 was obtained as a freeze-dried pellet from ATCC. Media for vegetative growth and antibiotic production were used as described (Lambalot et al., 1992). Briefly, SGGP liquid medium was for propagation of S. venezuelae mycelia. Sporulation agar (SPA) was used for production of S. venezuelae spores. Methymycin production was conducted in either SCM or vegetative medium and pikromycin production was performed in Suzuki glucose-peptone medium.

Vectors, DNA Manipulation and Cosmid Library Construction. pUC119 was the routine cloning vector, and pNJ1 was the cosmid vector used for genomic DNA library construction. Plasmid vectors for gene disruption were either pGM160 (Muth et al., 1989) or pKC1139 (Bierman et al., 1992). Plasmid, cosmid, and genomic DNA preparation, restriction digestion, fragment isolation, and cloning were performed using standard procedures (Sambrook et al., 1989; Hopwood et al., 1985). The cosmid library was made according to instructions from the Packagene λ-packaging system (Promega).

DNA Sequencing and Analysis. An Exonuclease III (ExoIII) nested deletion series combined with PCR-based double stranded DNA sequencing was employed to sequence the pik cluster. The ExoIII procedure followed the Erase-a-Base protocol (Stratagene) and DNA sequencing reactions were performed using the Dye Primer Cycle Sequencing Ready Reaction Kit (Applied Biosystems). The nucleotide sequences were read from an ABI PRISM 377 sequencer on both DNA strands. DNA and deduced protein sequence analyses were performed using GeneWorks and GCG sequence analysis package. All analyses were performed using the specific program default parameters.

Gene Disruption. A replicative plasmid-mediated homologous recombination approach was developed to conduct gene disruption in S. venezuelae. Plasmids for insertional inactivation were constructed by cloning a kanamycin resistance marker into target genes, and plasmid for gene deletion/replacement was constructed by replacing the target gene with a kanamycin or thiostrepton resistance gene in the plasmid. Disruption plasmids were introduced into S. venezuelae by either PEG-mediated protoplast transformation (Hopwood et al., 1985) or RK2-mediated conjugation (Bierman et al., 1992). Then, spores from individual transformants or transconjugants were cultured on non-selective plates to induce recombination. The cycle was repeated three times to enhance the opportunity for recombination. Double crossovers yielding targeted gene disruption mutants were selected and screened using the appropriate combination of antibiotics and finally confirmed by Southern hybridization.

Antibiotic Extraction and Analysis. Methymycin, pikromycin, and related compounds were extracted following published procedures (Cane et al., 1993). Thin layer chromatography (TLC) was routinely used to detect methymycin, neomethymycin, narbomycin and pikromycin. Further purification was conducted using flash column chromatography and HPLC, and the purified compounds were analyzed by ¹H, ¹³C NMR spectroscopy and MS spectrometry.

Results

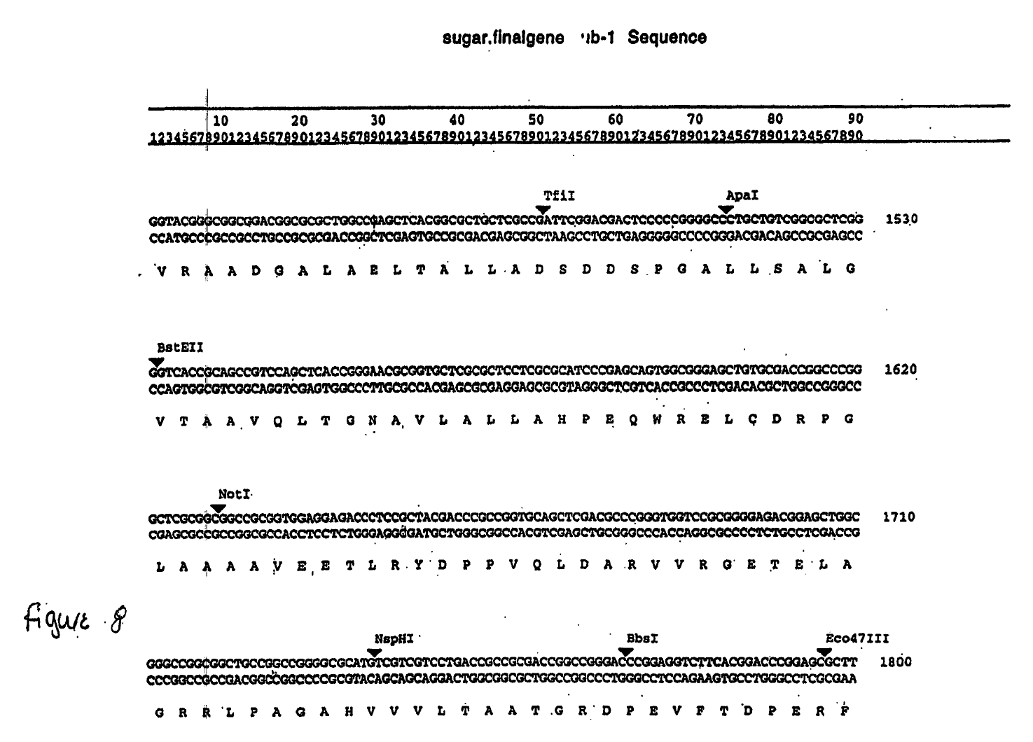

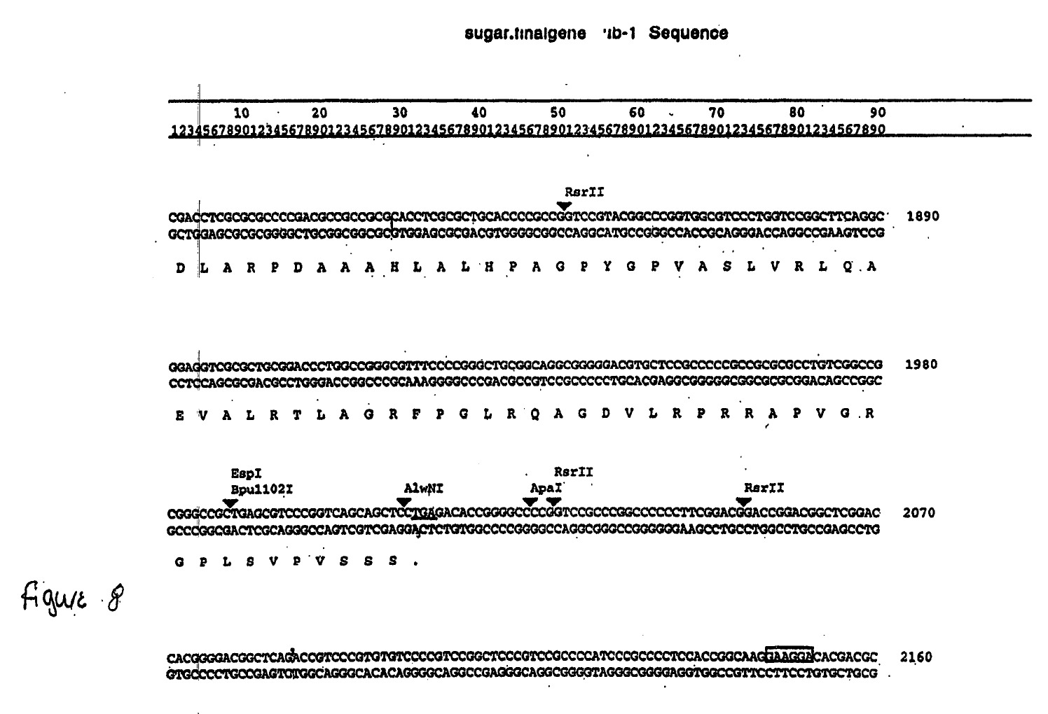

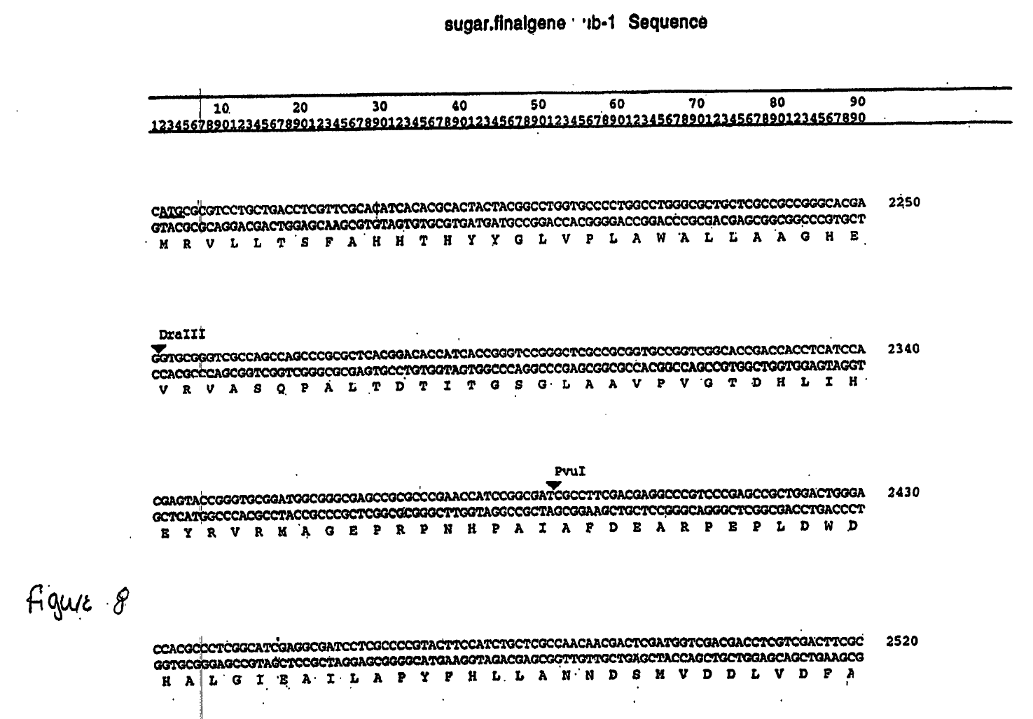

Cloning and Identification of the pik Cluster. Heterologous hybridization was used to identify genes for methymycin, neomethymycin, narbomycin and pikromycin biosynthesis in S. venezuelae. Initial Southern blot hybridization analysis using a type I PKS DNA probe revealed two multifunctional PKS clusters of uncharacterized function in the genome. Since these four antibiotics are all comprised of an identical desosamine residue, a tylAI α-D-glucose-1-phosphate thymidylyltransferase DNA probe (for mycaminose/mycorose/mycinose biosynthesis in the tylosin pathway) (Merson-Davies et al., 1994) was used to locate the corresponding biosynthetic gene cluster(s). This analysis established that only one of the PKS pathways contained a cluster of desosamine biosynthetic genes. Nine overlapping cosmid clones were isolated spanning over 80 kilobases (kb) on the bacterial chromosome that encompassed the entire gene cluster pik) for methymycin, neomethymycin, narbomycin and pikromycin biosynthesis (FIG. 5). Through subsequent gene disruption, the other PKS cluster (vep, devoid of linked desosamine biosynthetic genes) was found to play no role in production of methymycin, neomethymycin, narbomycin or pikromycin.

Nucleotide Sequence of the pik Cluster. The nucleotide sequence of the pik cluster was completely determined and shown to contain 18 open reading frames (ORFs) that span approximately 60 kb. Central to the cluster are four large ORFs, pikAI, pikAII, pikAIII, and pikAIV, encoding a multifunctional PKS (FIG. 5). Analysis of the six modules comprising the pik PKS indicated that it would specify production of narbonolide, the 14-membered ring aglycone precursor of narbomycin and pikromycin (FIG. 5).

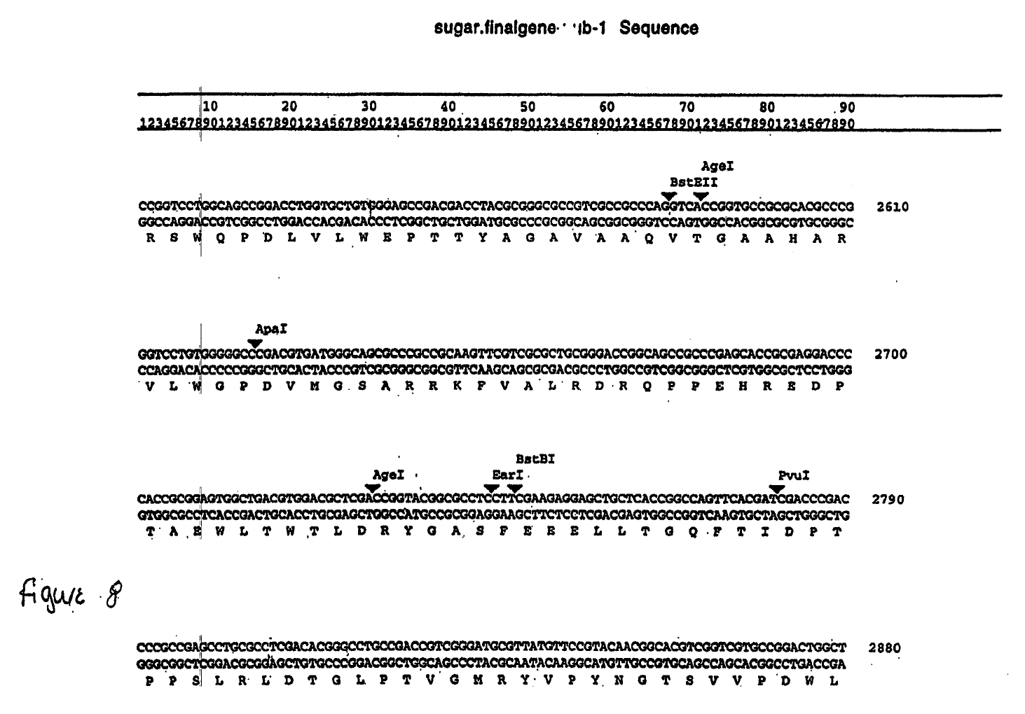

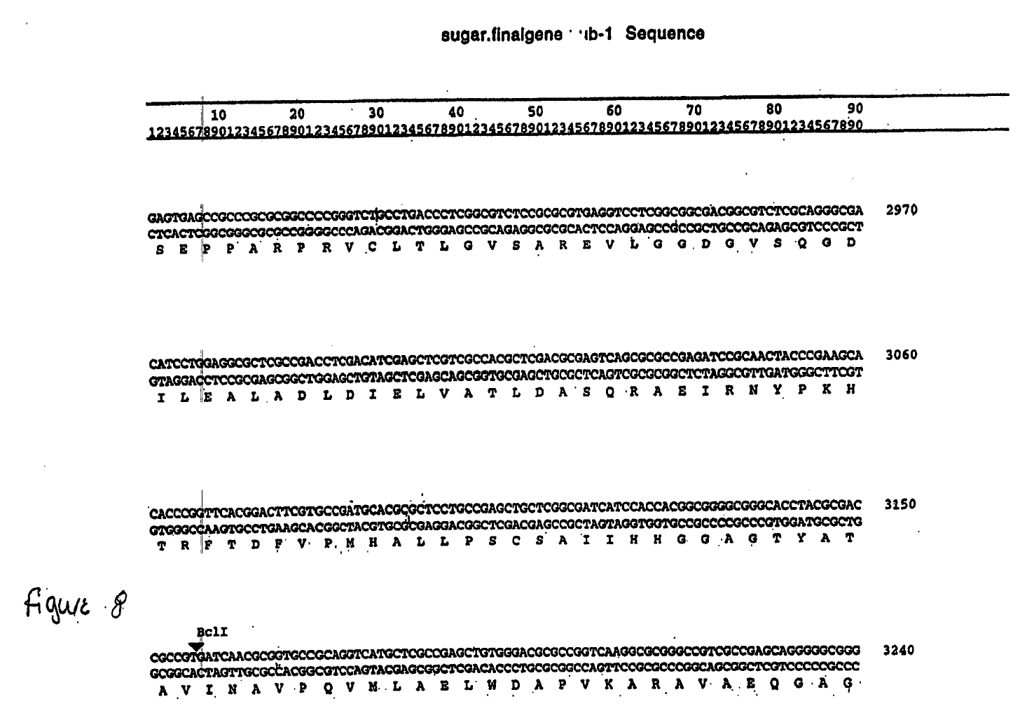

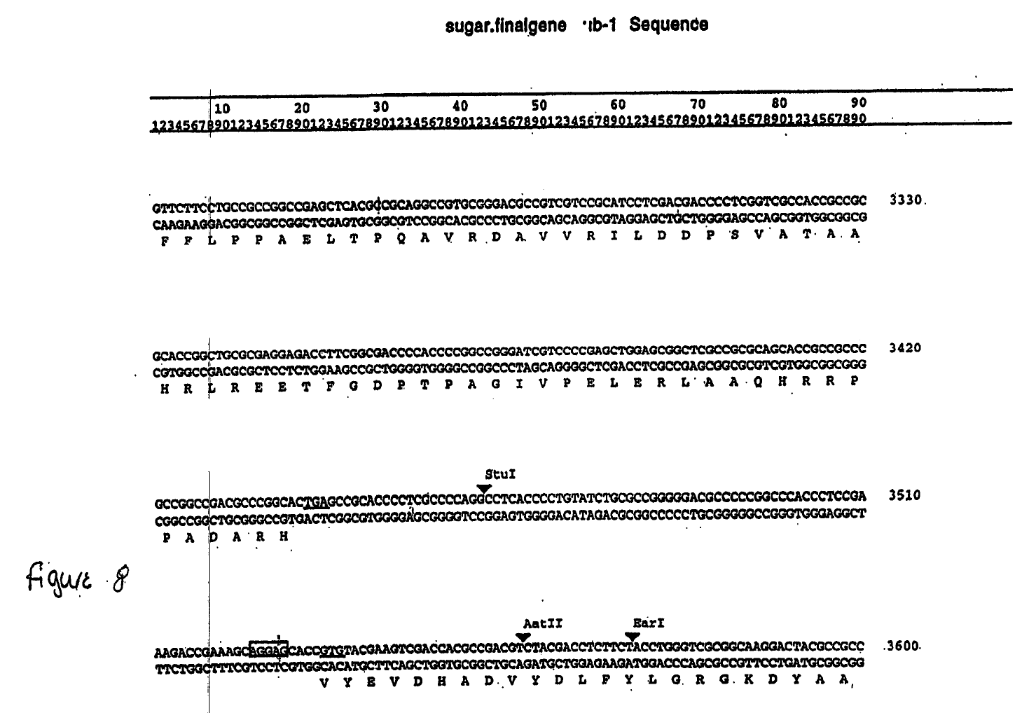

Initial analysis unveiled two significant architectural differences in the pik-A-encoded PKS. First, compared with eryA (Donadio et al., 1998) and oleA (Swan et al., 1994), two PKS clusters that produce 14-membered ring macrolides erythromycin and oleadomycin similar to pikromycin, the presence of separate ORFs, pikAIII and pikAIV, encoding Pik module 5 and Pik module 6 (as individual modules) as opposed to one bimodular protein as in eryAIII and oleAIII is striking. Secondly, the presence of a type II thioesterase immediately downstream of the type I PKS cluster is also unprecedented (FIG. 5). These two characteristics suggest that pikA may produce the 12-membered ring macrolactone 10-deoxymethynolide as well. Indeed, the domain organization of PikAI-AIII (module L-5) is consistent with the predicted biosynthesis of 10-deoxymethynolide except for the absence of a TE function at the C-terminus of Pik module 5 (PikAIII). The lack of a TE domain in PikAIII may be compensated by the type II TE (encoded by pikAV) immediately downstream of pikAIV. Consistent with the supposition that two distinct polyketide ring systems are assembled from the pik PKS, two macrolide-lincosamide-streptogramin B type resistant genes, pikR1 and pikR2, are found upstream of the pik PKS (FIG. 6), which presumably provide cellular self-protection for S. venezuelae.

The genetic locus for desosamine biosynthesis and glycosyl transfer are immediately downstream of pik4. Seven genes, desI, desII, desIII, desIV, desV, desVI, and desVIII, are responsible for the biosynthesis of the deoxysugar, and the eighth gene, desVII, encodes a glycosyltransferase that apparently catalyzes transfer of desosamine onto the alternate (12- and 14-membered ring) polyketide aglycones. The existence of only one set of desosamine genes indicates that DesVIII can accept both 10-deoxymethynolide and narbonolide as substrates (Jacobsen et al., 1997). The largest ORF in the des locus, desR, encodes a β-glycosidase that is involved in a drug inactivation-reactivation cycle for bacterial self-protection.

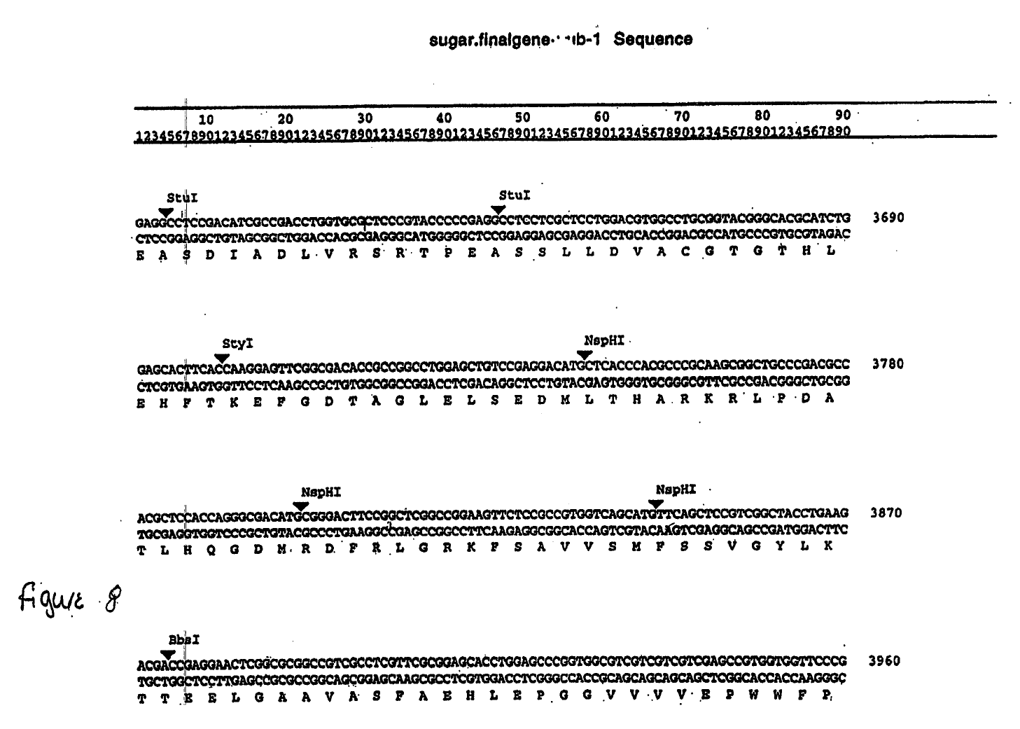

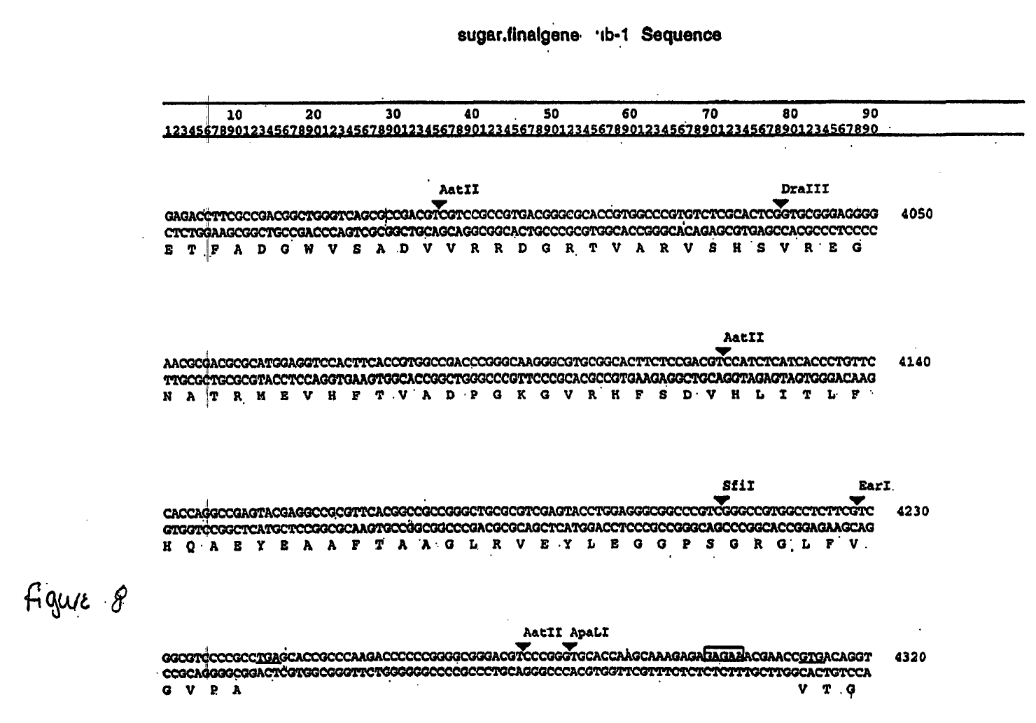

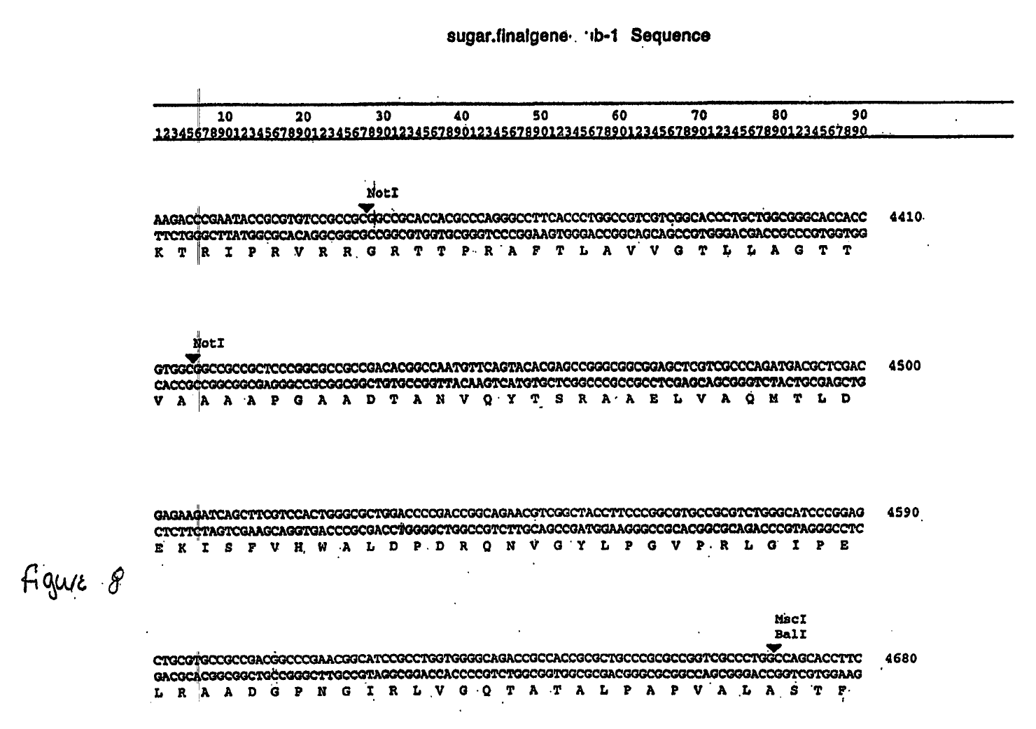

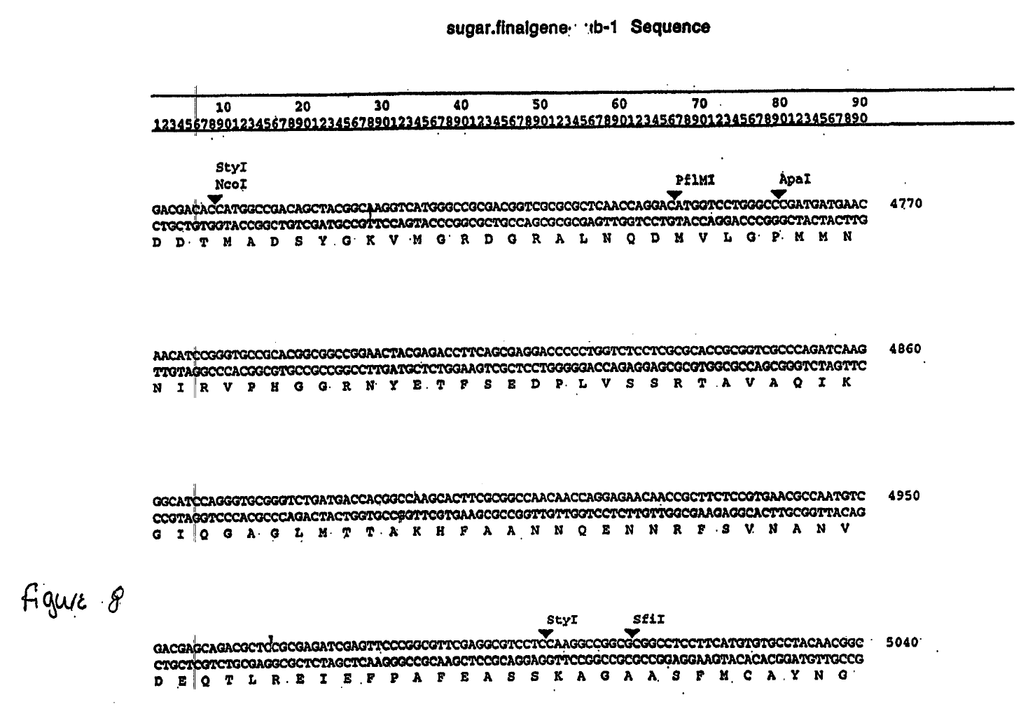

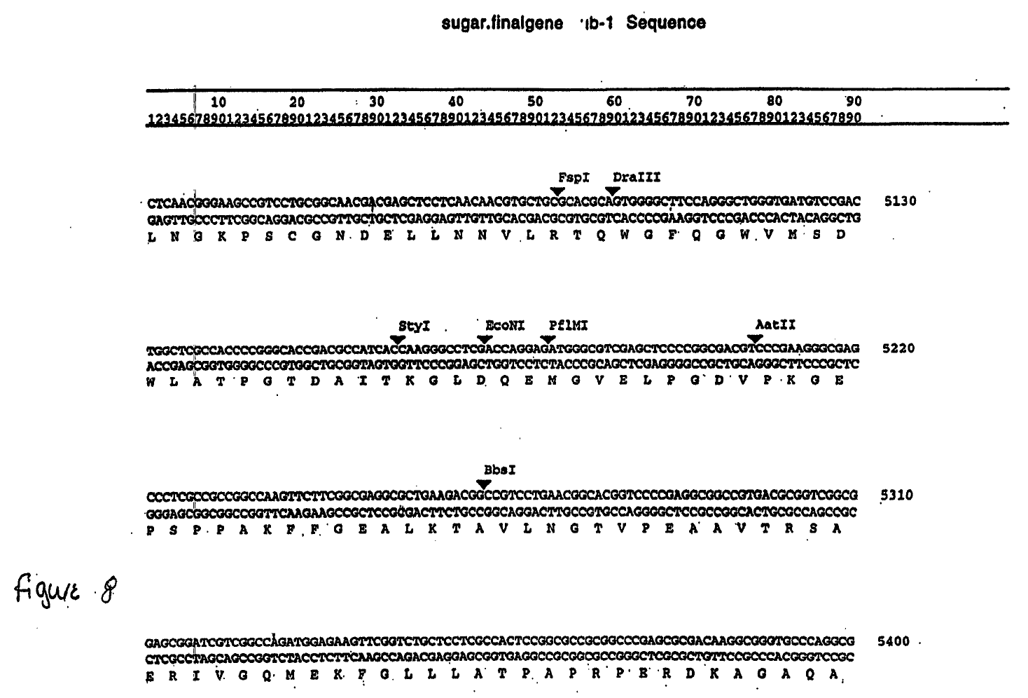

Just downstream of the des locus is a gene (pikC) encoding a cytochrome P450 hydroxylase similar to eryF (Andersen et al., 1992), and eryK (Stassi et al., 1993), PikC, and a gene (pikD) encoding a putative regulator protein, PikD (FIG. 5). Interestingly, PikC is the only P450 hydroxylase identified in the entire pik cluster, suggesting that the enzyme can accept both 12- and 14-membered ring macrolide substrates and, more remarkably, it is active on both C-10 and C-12 of the YC-17 (12-membered ring intermediate) to produce methymycin and neomethymycin (FIG. 7). PikD is a putative regulatory protein similar to ORFH in the rapamycin gene cluster (Schwecke et al., 1995).

The combined functionality coded by the eighteen genes in the pik cluster predicts biosynthesis of methymycin, neomethymycin, narbomycin and pikromycin (Table 1). Flanking the pik cluster locus are genes presumably involved in primary metabolism and genes that may be involved in both primary and secondary metabolism. An S-adenosyl-methionine synthase gene is located downstream of pikD that may help to provide the methyl group in desosamine synthesis. A threonine dehydratase gene was identified upstream of pikR1 that may provide precursors for polyketide biosynthesis. It is not apparent that any of these genes are dedicated to antibiotic biosynthesis and they are not directly linked to the pik cluster.

Deduced function of ORFs in the pik cluster

| | Amino acids, | Proposed function or | | | | | | |
|---|---|---|---|---|---|---|---|---|
| Polypeptide (ORF) | no. | sequence similarity detected | | | | | | |
| PikAI | 4,613 | PKS | | | | | | |
| Loading module | | KS^{Q} | AT(P) | | | | ACP | |
| Module 1 | | KS | AT(P) | | | KR | ACP | |
| Module 2 | | KS | AT(A) | DH | | KR | ACP | |
| PikAII | 3,739 | PKS | | | | | | |
| Module 3 | | KS | AT(P) | | | KR⁰ | ACP | |
| Module 4 | | KS | AT(P) | DH | ER | KR | ACP | |
| PikAIII | 1,562 | PKS | | | | | | |
| Module 5 | | KS | AT(P) | | | KR | ACP | |
| PikAIV | 1,346 | PKS | | | | | | |
| Module 6 | | KS | AT(P) | | | | ACP | TE |

| | | |
|---|---|---|
| PikAV | 281 | Thioesterase II (TEII) |
| DesI | 415 | 4-Dehydrase |
| DesII | 485 | Reductase |
| DesIII | 292 | α-D-Glucose-1-phosphate thymidylyltransferase |
| DesIV | 337 | TDP-glucose 4,6-dehydratase |
| DesV | 379 | Transaminase |
| DesVI | 237 | N,N-dimethyltransferase |
| DesVII | 426 | Glycosyl transferase |
| DesVIII | 402 | Tautomerase |
| DesR | 809 | β-Glucosidase (involved in resistance |
| | | mechanism) |
| PikC | 418 | P450 hydroxylase |
| PikD | 945? | Putative regulator |
| PikR1 | 336 | rRNA methyltransferase (mls resistance) |
| PikR2 | 288? | rRNA methyltransferase (mls resistance) |

AT(A), acyltransferase incorporating an acetate extender unit;
AT(P), acyltransferase incorporating a propionate extender unit.
KR⁰, an inactive KR.
Enzymes of uncertain function are denoted with a question mark.

TABLE 2
Summary of mutational analyses of the pik cluster

| | | | Antibiotic production/ | |
|---|---|---|---|---|
| | Type of | Target | Intermediate accumulation | |
| Mutant | mutation | gene | Met & neomethymycin | Pikromycin |
| AX903 | Insertion | pikAI | No/No | No/No |
| LZ3001 | Deletion/ | desVI | No/10- | No/narbonolide |
| | replacement | | deoxymethynolide | |
| LZ4001 | Deletion/ | desV | No/10- | No/narbonolide |
| | replacement | | deoxymethynolide | |
| AX905 | Deletion/ | pikAV | <5%/No | <5%/No |
| | replacement | | | |
| AX906 | Insertion | pikC | No/YC-17 | No/narbomycin |

Mutational Analysis of the pik Cluster. Extensive disruption of genes in the pik cluster were carried out to address the role of key enzymes in antibiotic production (Table 2). First, PikAI, the first putative enzyme involved in the biosynthesis of 10-deoxymethynolide and narbonolide was inactivated by insertional mutagenesis. The resulting mutant, AX903, produced neither methymycin or neomethymycin, nor narbomycin or pikromycin, indicating that pikA encodes a PKS required for both 12- and 14-membered ring macrolactone formation.

Second, deletion of both desVI and des V abolished methymycin, neomethymycin, narbomycin and pikromycin production, and the resulting mutants, LZ3001 and LZ4001, accumulate 10-deoxymethynolide and narbonolide in their culture broth, indicating that enzymes for desosamine synthesis and transfer are also shared by the 12- and 14-membered ring macrolides.

In order to understand the mechanism of polyketide chain termination at PikAIII (PIKAIII (module 5) is presumed to be the termination point in construction of 10-deoxymethynolide), the pik TEII gene, pikAV, was deleted. The deletion/replacement mutant, AX905, produces less than 5% of methymycin, neomethymycin, and less than 5% of pikromycin compared to wild type S. venezuelae. This abrogation in product formation occurs without significant accumulation of the expected aglycone intermediates, suggesting that pik TEII is involved in the termination of 12- as well as 14-membered ring macrolides at PikAIII and PikAIV, respectively. Although the polar effects may influence the observed phenotype in AX905, this has been ruled out after the consideration of mutant LZ3001, in which mutation in an enzyme downstream of pikAV accumulated 10-deoxymethynolide and narbonolide. The fact that mutant AX905 failed to accumulate these intermediates suggested that the polyketide chains were not efficiently released from this PKS protein in the absence of Pik TEII. Therefore, Pik TEII plays a crucial role in polyketide chain release and cyclization, and it presumably provides the mechanism for alternative termination in pik polyketide biosynthesis.

Finally, disruption of pikC confirmed that PikC is the sole enzyme catalyzing hydroxylation of both YC-17 (at C-10 and C-12) and narbomycin (at C-12). The relaxed substrate specificity of PikC and its regional specificity at C-10 and C-12 provide another layer of metabolite diversity in the pik-encoded biosynthetic system.

Discussion

The work described herein has established that methymycin, neomethymycin, narbomycin and pikromycin biosynthesis is encoded by the pik cluster in S. venezuelae. Three key enzymes as well as the unique architecture of the cluster enable this relatively compact system to produce multiple macrolide antibiotics. Foremost, the presence of pik module 5 and 6 as separate proteins, pikAIII and PikAIV, and the activity of pik TEII enable the bacterium to terminate the polyketide chain at two different points of assembly, thereby producing two macrolactones of different ring size. Second, DesVII, the glycosyltransferase in the pik cluster, can accept both 12- and 14-membered ring macrolactones as substrates. Finally, PikC, the P450 hydroxylase, has a remarkable substrate and regiochemical specificity that introduces another layer of diversity into the system.

It is interesting to consider that pikA evolved in a line analogous to eryA and oleA since each of these PKSs specify the synthesis of 14-membered ring macrolactones. Therefore, pik may have acquired the capacity to generate methymycin when a mutation in the primordial pikAII-pikAIV linker region caused splitting of Pik module 5 and 6 into two separate gene products. This notion is raised by two features of the nucleotide sequence. First, the intergenic region between pikAIII and pikAIV, which is 105 bp, may be the remanent of an intramodular linker peptide of 35 amino acids. Moreover, the potential for independently regulated expression of pikAIV is implied by the presence of a 100 nucleotide region at the 5′ end of the gene that is relatively AT-rich (62% as comparing 74% G+C content in coding region). Thus, as the mutation in an original ORF encoding the bimodular multifunctional protein (PikAIII-PikAIV) occurred, so too may have evolved a mechanism for regulated synthesis of the new gene product (PikAIV).

The role of Pik TEII in alternative termination of polyketide chain elongation intermediates provides a unique aspect of diversity generation in natural product biosynthesis. Engineered polyketides of different chain length are typically generated by moving the TE catalytic domain to alternate positions in a modular PKS (Cortes et al., 1995). Repositioning of the TE domain necessarily abolishes production of the original full-length polyketide so only one macrolide is produced each time. In contrast to the fixed-position TE domain, the independent Pik TEII polypeptide presumably has the flexibility to catalyze termination at different stages of polyketide assembly, therefore enabling the system to produce multiple products of variant chain length. Combinatorial biology technologies can now exploit this system for generating molecular diversity through construction of novel PKS systems with TEIIs for simultaneous production of several new molecules as opposed to the TE domains alone that limit catalysis to a single termination step.

It is noteworthy that sequences similar to Pik TEII are found in almost all known polyketide and non-ribosomal polypeptide biosynthetic systems (Narahiel et al., 1997). Currently, the pik TEII is the first to be characterized in a modular PKS. However, recent work on a TEII gene in the lipopeptide surfactin biosynthetic cluster (Schneider et al., 1998) demonstrated that srf-TEII plays an important role in polypeptide chain release, and may suggest that srf-TEII reacts at multiple stages in peptide assembly as well (Marahiel et al., 1997).

The enzymes involved in post-polyketide assembly of 10-deoxymethynolide and narbonolide are particularly intriguing, especially the glycosyltransferase, Des VII, and P450 hydroxylase, PikC. Both have the remarkable ability to accept substrates with significant structural variability. Moreover, disruption of desVI demonstrated that DesVII also tolerates variations in deoxysugar structure. Likewise, PikC has recently been shown to convert YC-17 to methymycin/neomethymycin and narbomycin to pikromycin in vitro.

Targeted gene disruption of ORF1 abolished both pikromycin and methymycin production, indicating that the single cluster is responsible for biosynthesis of both antibiotics. Deletion of the TE2 gene substantially reduced methymycin and pikromycin production, which demonstrates that TE2, in contrast to the position-fixed TE1 domain, has the capacity to release polyketide chain at different points during the assembly process, thereby producing polyketides of different chain length.

The results described above were unexpected in that it was surprising that one PKS cluster produces two macrolides which differ in the number of atoms in their ring structure, that module 5 and module 6 of the PKS are in ORFs that are separated by a spacer region, that PikAIII lacked TE, that there was a Type II thioesterase, that TEI domain was not separate, and that 2 resistance genes were identified which may be specific for either a 12- or 14-membered ring.

With eighteen genes spanning less than 60 kb of DNA capable of producing four active macrolide antibiotics, the pik cluster represents the least complex yet most versatile modular PKS system so far investigated. This simplicity provides the basis for a compelling expression system in which novel active ketoside products are engineered and produced with considerable facility for discovery of a diverse range of new biologically active compounds.

Summary

Complex polyketide synthesis follows a processive reaction mechanism, and each module within a PKS harbors a string of three to six enzymatic domains that catalyze reactions in nearly linear order as described in particular detail for the erythromycin-producing PKS (Katz, 1997; Khosla, 1997; Staunton et al. 1997). The combined set of PKS modules and catalytic domains along with genes that encode enzymes for post-polyketide tailoring (e.g., glycosyl transferases, hydroxylases) typically limits a biosynthetic system to the generation of a single polyketide product.

Combinatorial biology involves the genetic manipulation of multistep biosynthetic pathways to create molecular diversity in natural products for use in novel drug discovery. PKSs represent one of the most amenable systems for combinatorial technologies because of their inherent genetic organization and ability to produce polyketide metabolites, a large group of natural products generated by bacteria (primarily actinomycetes and myxobacteria) and fungi with diverse structures and biological activities. Complex polyketides are produced by multifunctional PKSs involving a mechanism similar to long-chain fatty acid synthesis in animals (Hopwood et al., 1990). Pioneering studies (Cortes et al., 1990; Donadio et al., 1991) on the erythromycin PKS in Saccharopolyspora erythraea revealed a modular organization. Characterization of this multidomain protein system, followed by molecular analysis of rapamycin (Aparicio et al., 1996), FK506 (Motamedi et al., 1997), soraphen A (Schupp et al., 1995), niddamycin (Kakavas et al., 1997), and rifamycin (August et al., 1998) PKSs, demonstrated a co-linear relationship between modular structure of a multifunctional bacterial PKS and the structure of its polyketide product.

In a survey of microbial systems capable of generating unusual metabolite structural variability, Streptomyces venezuelae ATCC 15439 is notable in its ability to produce two distinct groups of macrolide antibiotics. Methymycin and neomethymycin are derived from the 12-membered ring macrolactone 10-deoxymethynolide, while narbomycin and pikromycin are derived from the 14-membered ring macrolactone, narbonolide. The cloning and characterization of the biosynthetic gene cluster for these antibiotics reveals the key role of a type II thioesterase in forming a metabolic branch through which polyketides of different chain length are generated by the pikromycin multifunctional polyketide synthase (PKS). Immediately downstream of the PKS genes (pikA) are a set of genes for desosamine (des) biosynthesis and macrolide ring hydroxylation. The glycosyl transferase (encoded by desVIII) has the remarkable ability to catalyze glycosylation of both the 12- and 14-membered ring macrolactones. Moreover, the pikC-encoded P450 hydroxylase provides yet another layer of structural variability by introducing regiochemical diversity into the macrolide ring systems.

### EXAMPLE 4

### A desV Deletion Mutant Yields D-Quinovose

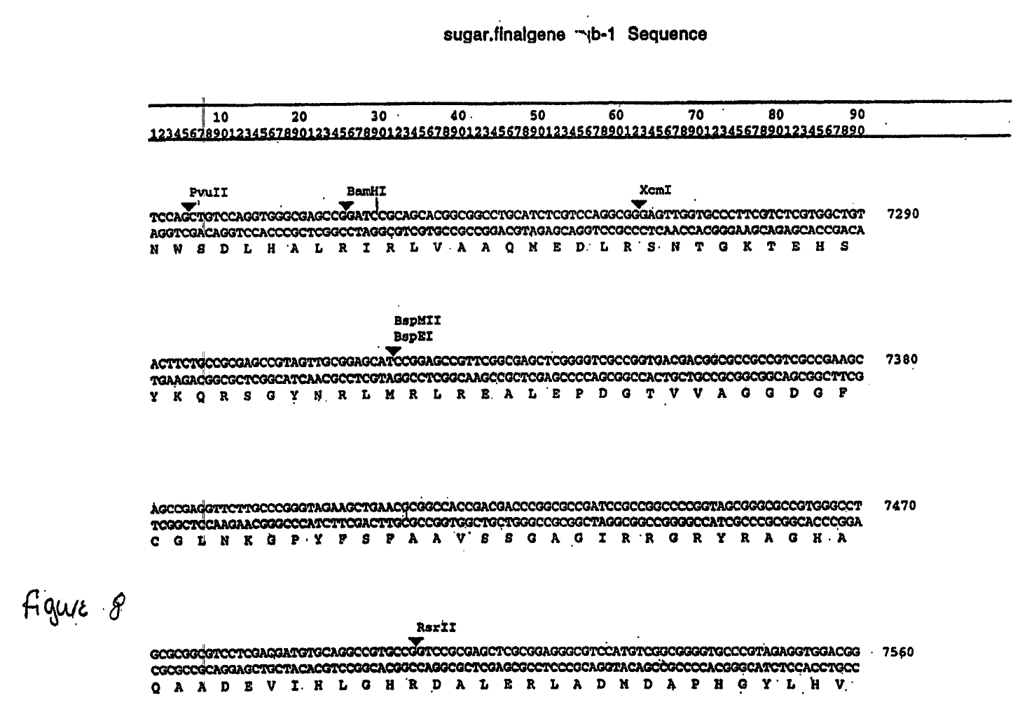

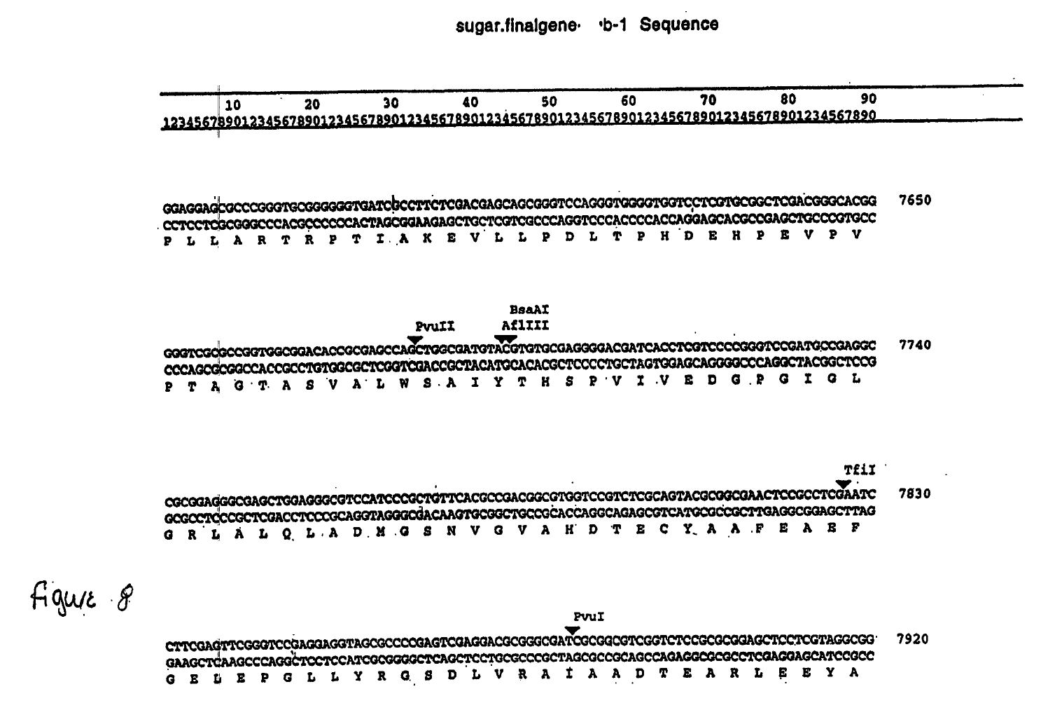

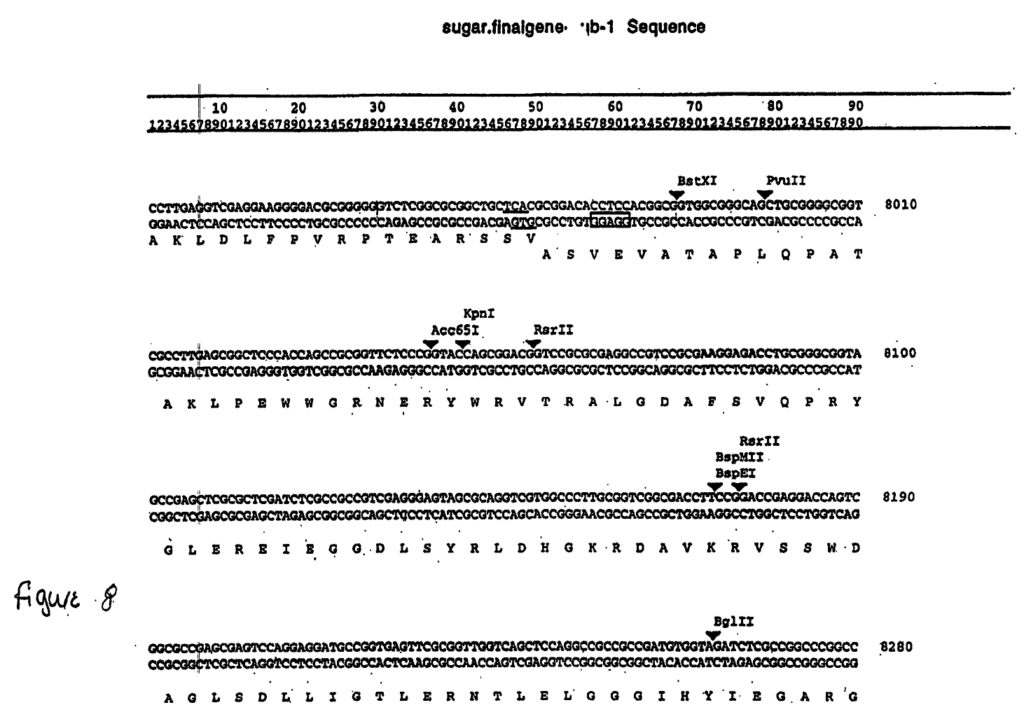

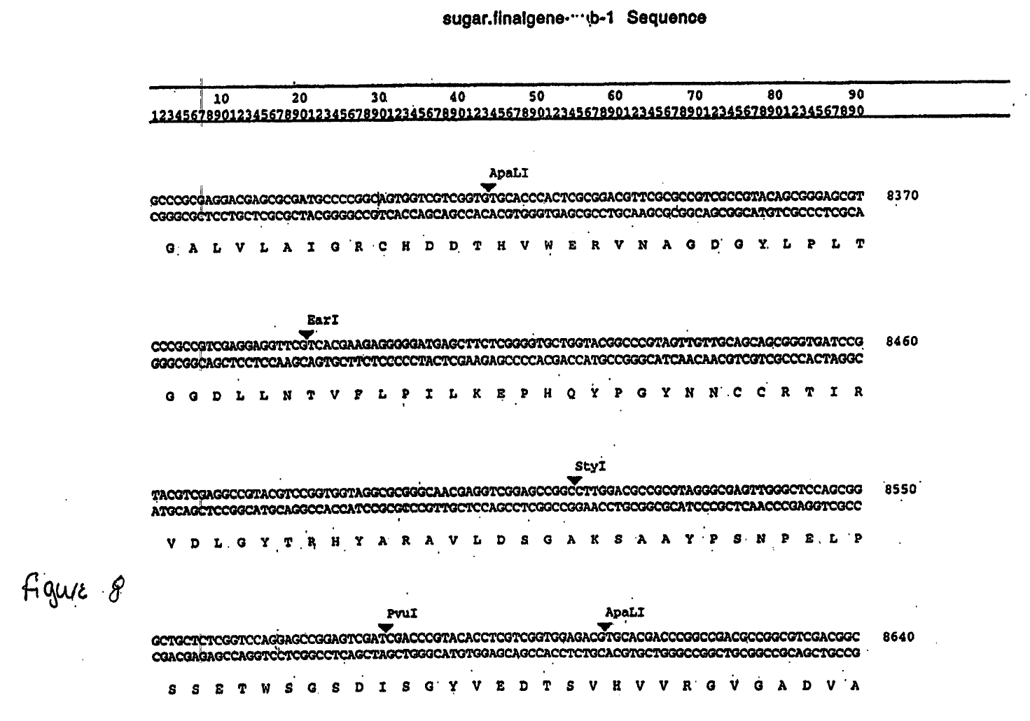

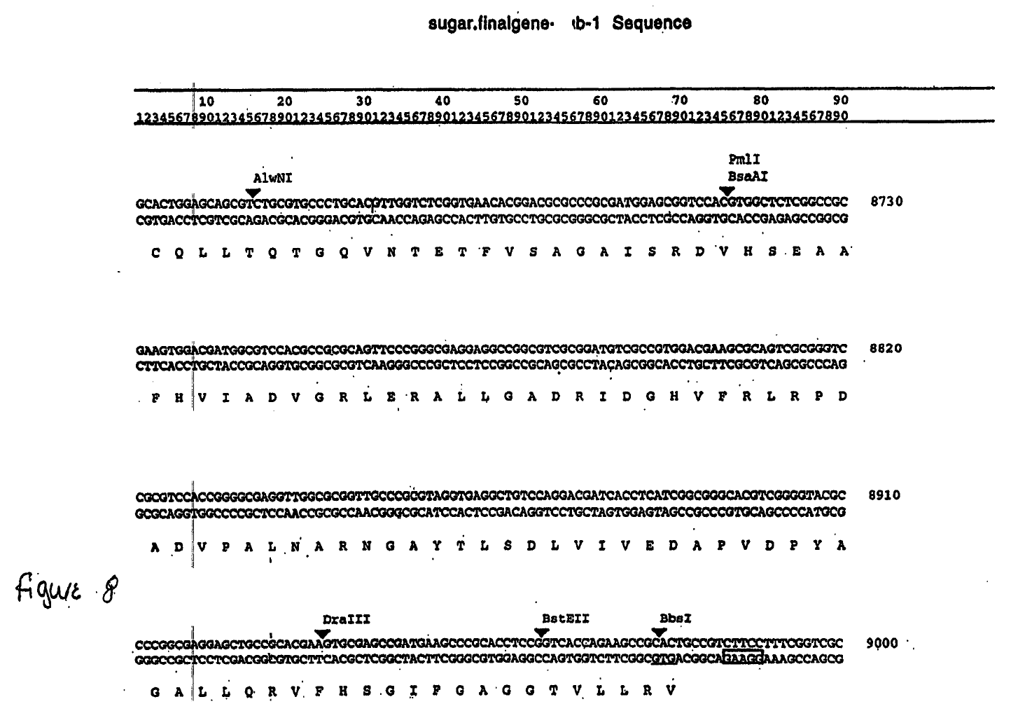

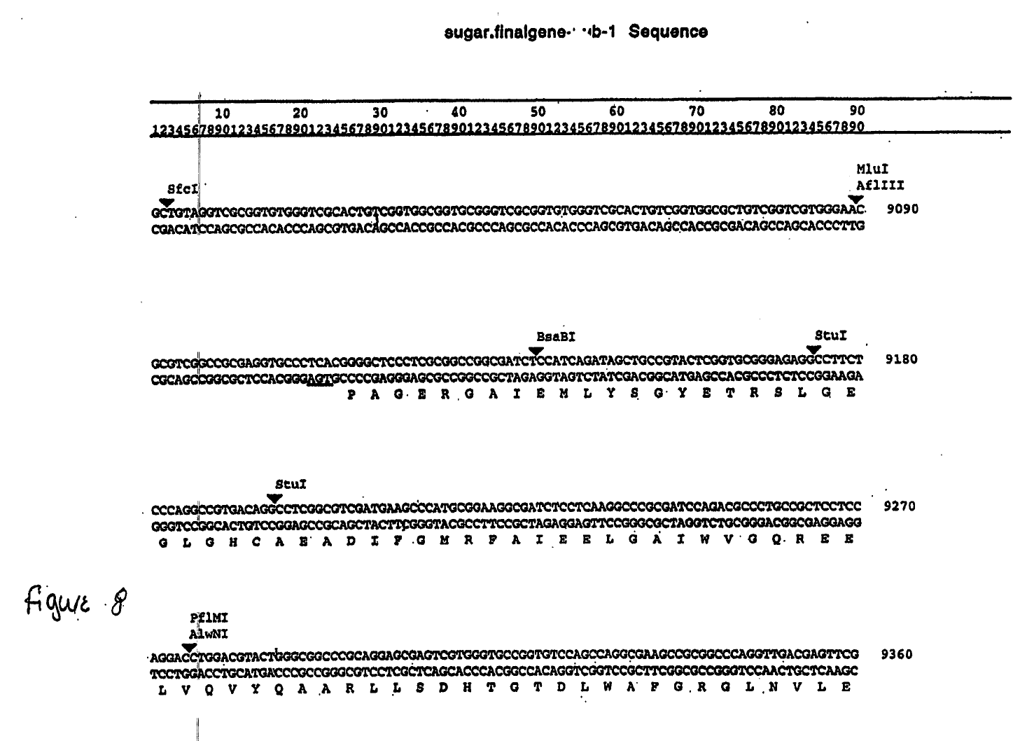

A mutant of S. venezuelae (KdesV-41) was constructed that had the desV gene disrupted (Zhao et al., J. Am. Chem. Soc., 120, 12159 (1998)). Since desV encodes the 3-aminotransferase that catalyzes the conversion of the 3-keto sugar 17 (FIG. 11) to the corresponding amino sugar 4, deletion of this gene should prevent C-3 transamination, resulting in the accumulation of 17. It was expected that if the glycosyltransferase (DesVII) of this pathway is capable of recognizing and processing the keto sugar intermediate 17, the macrolide product(s) produced by the KdesV-41 mutant should have an attached 3-keto sugar. Surprisingly, the two products isolated were the methymycin/neomethymycin analogues 18 and 19, each carrying a 4,6-dideoxyhexose (FIG. 12). While this result demonstrated a relaxed specificity for the glycosyltransferase toward its sugar substrate, it also indicated the existence of a pathway-independent reductase in S. venezuelae that can stereospecifically reduce the C-3 keto group of the sugar metabolite.

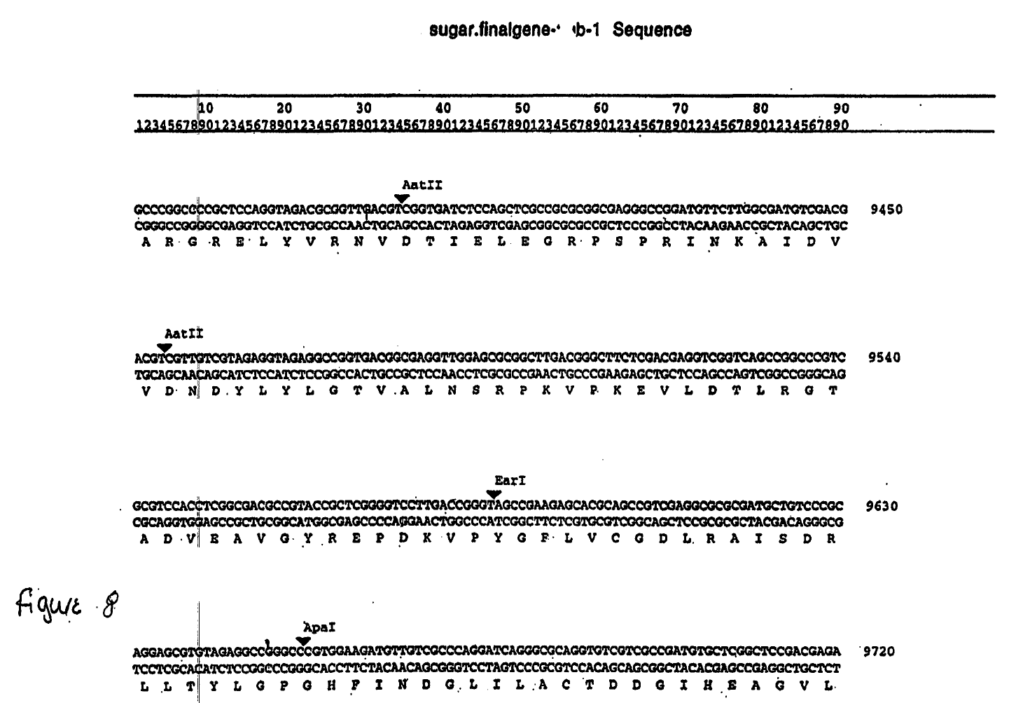

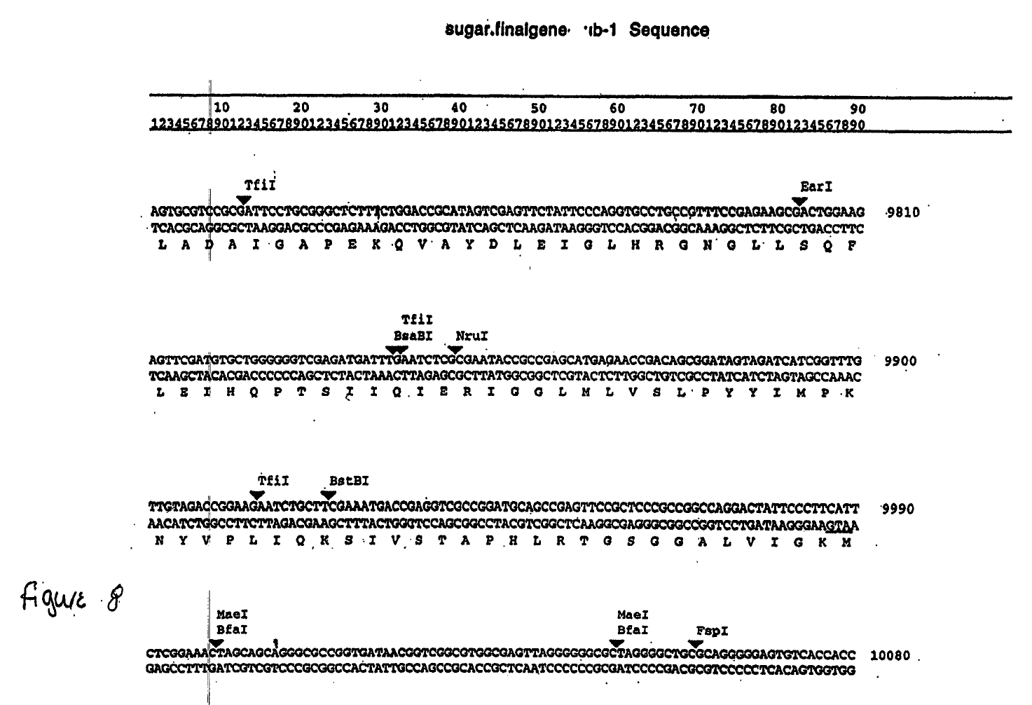

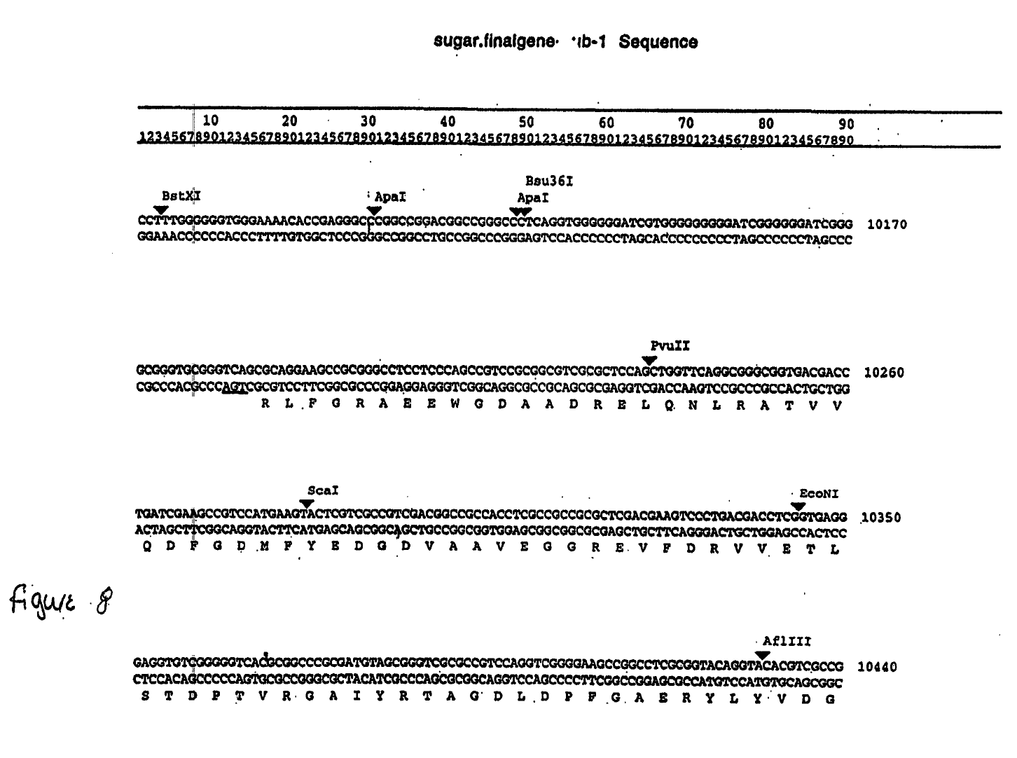

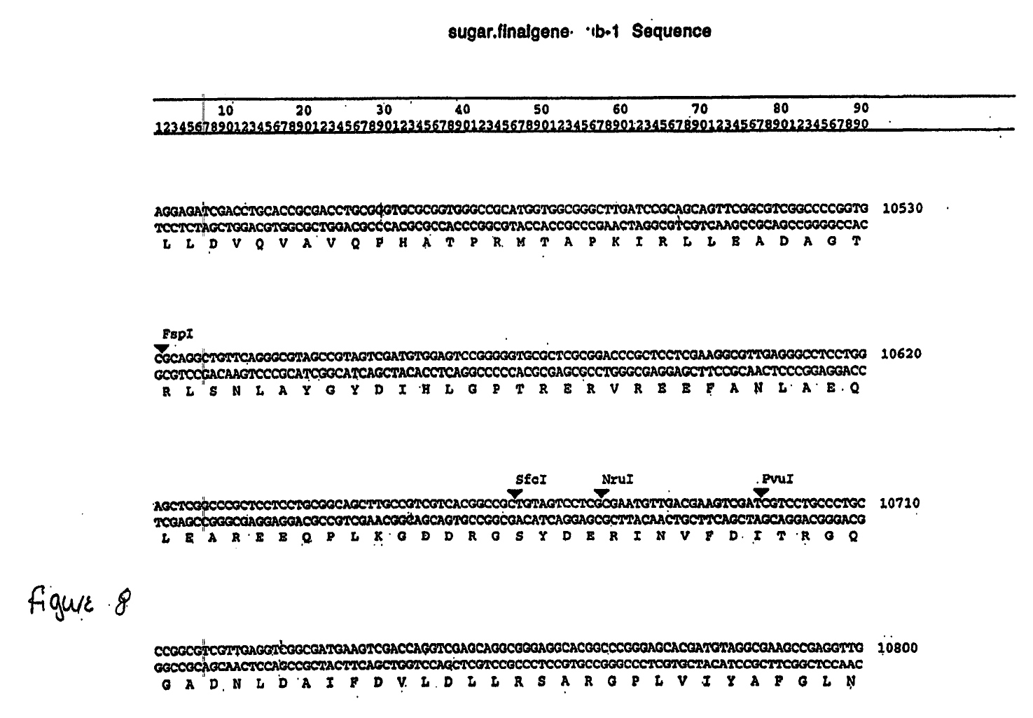

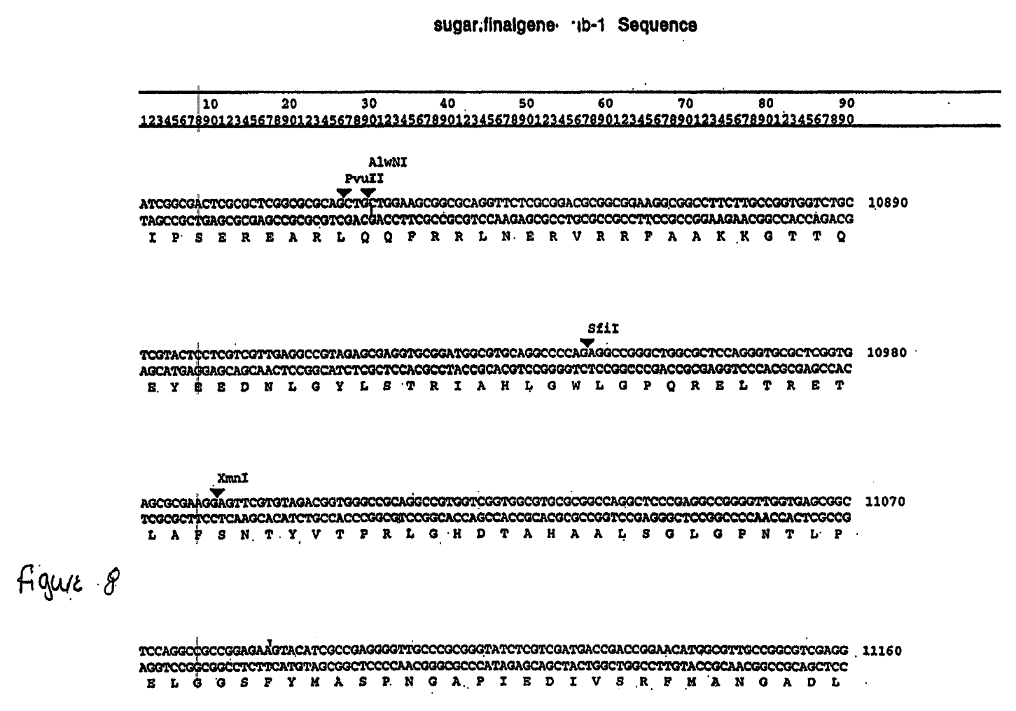

To explore the possibility of generating a mutant capable of synthesizing new macrolides of this class containing an engineered sugar, the desI gene, which has been proposed to encode the dehydrase responsible for the C-4 deoxygenation in the biosynthesis of desosamine, was altered with the prediction that it would lead to the incorporation of D-quinovose (22; FIG. 13), also known as 6-deoxy-D-glucose, into the final product(s). The rationale was based on the following: (1) Desosamine biosynthesis will be “terminated” at the C-4 deoxygenation step due to desI deletion and, thus, should result in the accumulation of 3-keto-6-deoxyhexose 16 (FIG. 11). (2) By taking advantage of the existence of a 3-ketohexose reductase in S. venezuelae, the sugar intermediate 15 is expected to be reduced stereospecifically to D-quinovose (22). (3) The glycosyltransferase (DesVII), with its relaxed specificity toward the sugar substrate, should catalyze the coupling of 22 to the macrolactones to give new macrolides 20 and 21 containing the engineered sugar D-quinovose (FIG. 13).

A disruption plasmid, pDesI-K, derived from pKC1139 that contains an apramycin resistant marker, was constructed in which desI was replaced by the neomycin resistance gene, which also confers resistance to kanamycin. This construct was then introduced into wild type S. venezuelae by conjugal transfer using Escherichia coli S17-1 as the donor strain (Bierman et al., 1992). Several double crossover mutants were identified on the basis of their phenotypes of kanamycin resistant (Kan^{R}) and apramycin sensitive (Apr^{S}). One mutant, KdesI-80, was selected and grown at 29° C. in seed medium (100 mL) for 48 hours and then inoculated and grown in vegetative medium (5 L) for another 48 hours (Cane et al., 1993). The fermentation broth was centrifuged to remove cellular debris and mycelia, and the supernatant was adjusted to pH 9.5 with concentrated potassium hydroxide solution. The resulting solution was extracted with chloroform, and the pooled organic extracts were dried over sodium sulfate and evaporated to dryness. The yellow oil was subjected to flash chromatography on silica gel using a gradient of 0-12% methanol in chloroform, and the isolated products were further purified by HPLC using a C₁₈ column eluted isocratically with 50% acetonitrile in water. As expected, no methymycin or neomethymycin was detected; instead, 10-deoxymethynolide 23 was found as the major product (approximately 600 mg). Significant quantities of methynolide 24 (approximately 40 mg) and neomethynolide 25 (approximately 2 mg) were also isolated (FIG. 13). A new macrolide 15 containing D-quinovose (3.2 mg) was produced by this mutant. Its structure was fully established by spectral analyses. Spectral data (J values are in hertz) for 15: ¹H NMR (CDCl₃) δ 6.76 (1H, dd, J=16.0, 5.5, 9-H), 6.43 (1H, d, J=16.0, 8-H), 4.97 (1H, ddd, J=8.4, 5.9, 2.5, 11-H), 4.29 (1H, d, J=8.0, 1′-H),3.62 (1H, d, J=10.5, 3-H), 3.49 (1H, t, J=9.0, 3′-H), 3.36 (1H, dd, J=9.0, 8.0, 2′-H), 3.32 (1H, dq, J=8.5, 5.5, 5′-H), 3.23 (1H, dd, J=9.0, 8.5, 4′-H), 2.82 (1H, dq, J=10.5, 7.0, 2-H), 2.64 (1H, m, 10-H), 2.55 (1H, m, 6-H), 1.70 (1H, m, 12a-H), 1.66 (1H, bt, J=12.5, 5b-H), 1.56 (1H, m, 12b-H), 1.40 (1H, dd, J=12.5, 4.5, 5a-H), 1.35 (3H, d, J=7.0, 2-Me), 1.31 (3H, d, J=5.5, 5′-Me), 1.24 (1H, bdd, J=10.0, 4.5, 4-H), 1.21 (3H, d, J=7.0, 6-Me), 1.11 (3H, d, J=6.5, 10-Me), 1.00 (3H, d, J=7.0, 4-Me), 0.92 (3H, t, J=7.5, 12-Me); ¹³C NMR (CDCl₃) δ 205.0 (C-7), 174.7 (C-1), 146.9 (C-9), 125.9 (C-8), 102.9 (C-1′), 85.4 (C-3′), 76.5 (C-3′), 75.5 (C-4′), 74.7 (C-2′), 73.9 (C-11), 71.6 (C-5′), 45.0 (C-6), 43.9 (C-2), 37.9 (C-10), 34.1 (C-5), 33.4 (C-4), 25.2 (C-12), 17.7 (6-Me), 17.5 (5′-Me), 17.4 (4-ME), 16.2 (2-Me), 10.3 (12-Me), 9.6 (10-Me); high-resolution FAB-MS calculated for C₂₃H₃₈O₈ (M+H)⁺ 443.2644, found 443.2661.

The fact that macrolide 15 containing D-quinovose is indeed produced by the desI mutant is significant. First, the formation of quinovose as predicted further corroborates the presence of a pathway-independent reductase in S. venezuelae that reduces the 3-keto sugars. Interestingly, this reductase is able to act on the 4,6-dideoxy sugar 17 as well as the 6-deoxy sugar 16, suggesting that it is oblivious to the presence of a hydroxyl group at C-4. However, it is not clear at this point whether the reduction occurs on the free sugar or after it is appended to the aglycone. Second, the retention of the 4-OH in quinovose as a result of desI deletion provides strong evidence supporting the assigned role of desI to encode a C-4 dehydrase. Moreover, the results again show that the glycosyltransferase (DesVII) of this pathway can recognize alternative sugar substrates whose structures are considerably different from the original amino sugar substrate desosamine. While the incorporation of quinovose is important, another noteworthy, albeit unexpected, result was the fact that the aglycone of the isolated macrolide 15 was 10-deoxy-methynolide 23 instead of methynolide 24 and neomethynolide 25. It is possible that the cytochrome P450 hydroxylase (PikC), which catalyzes the hydroxylation of 10-deoxy-methynolide at either its C-10 or C-12 position (Xue et al., 1998), is sensitive to structural variations in the appended sugar. It could be argued that the presence of the 4-OH group in the sugar moiety is somehow responsible for decreasing or preventing hydroxylation of the macrolide.

Thus, the results demonstrate the feasibility of combining pathway-dependent genetic manipulations and pathway-independent enzymatic reactions to engineer a sugar of designed structure. It is conceivable that the pathway-independent enzymes could also be used in concert with the natural biosynthetic machinery to generate further structural diversity, which can provide an array of random compounds.

### EXAMPLE 5

### Engineering a Hybrid Macrolide

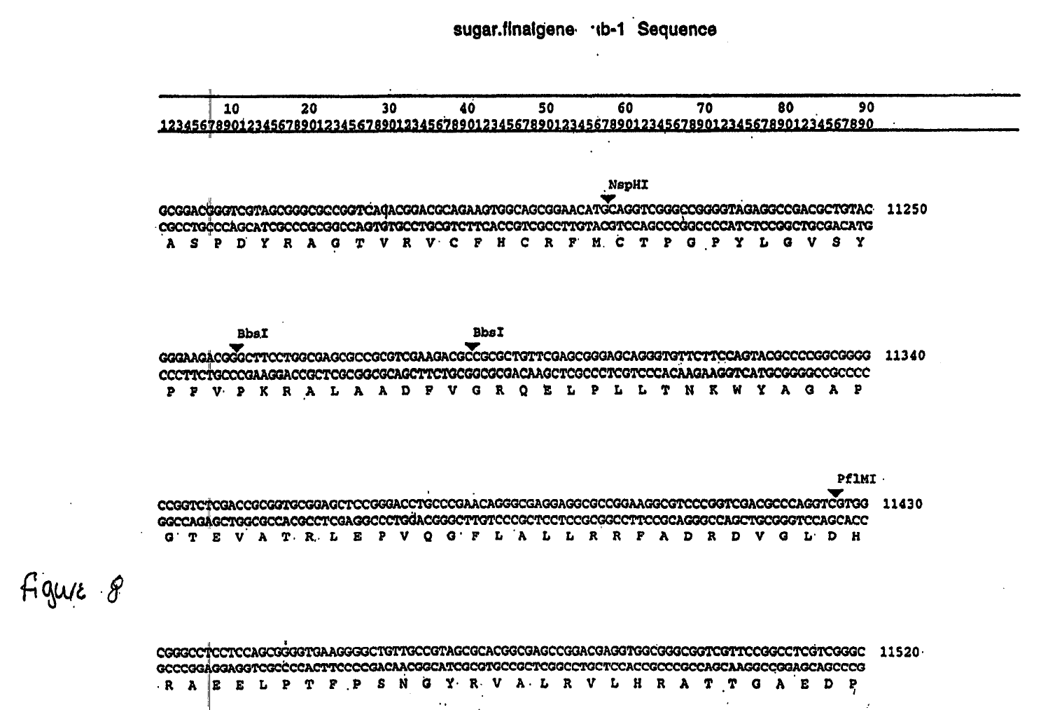

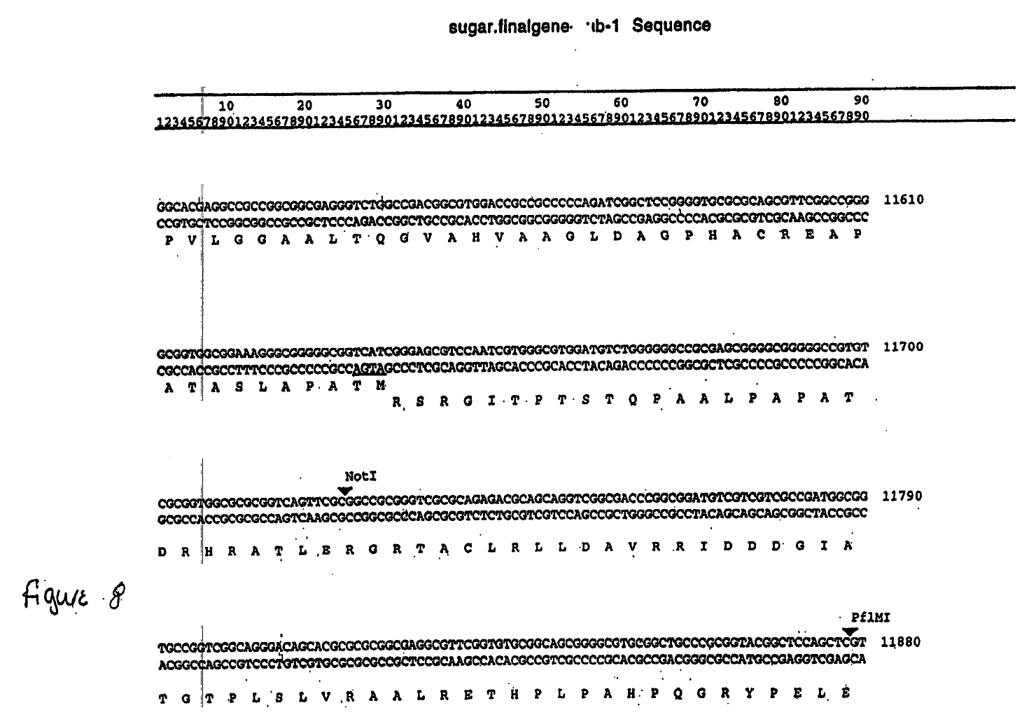

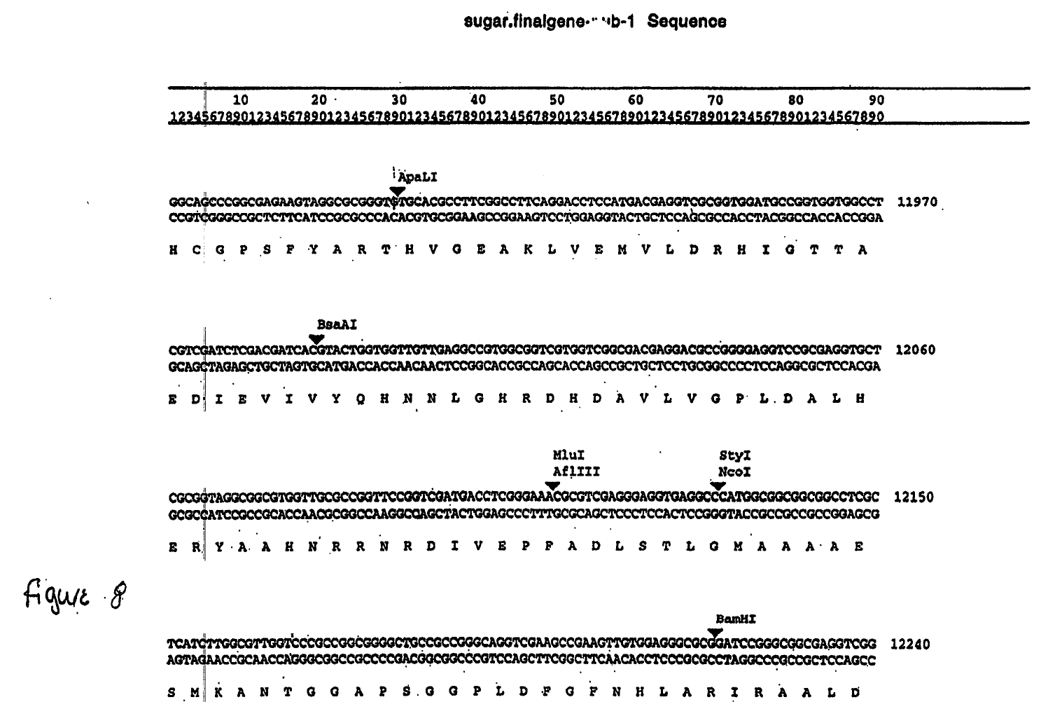

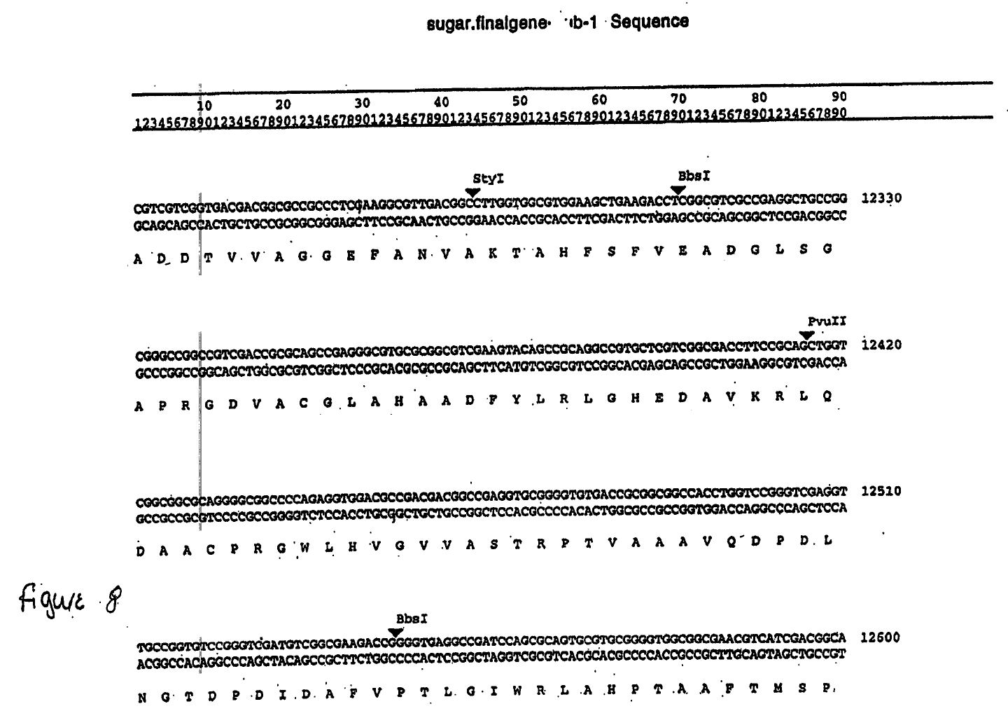

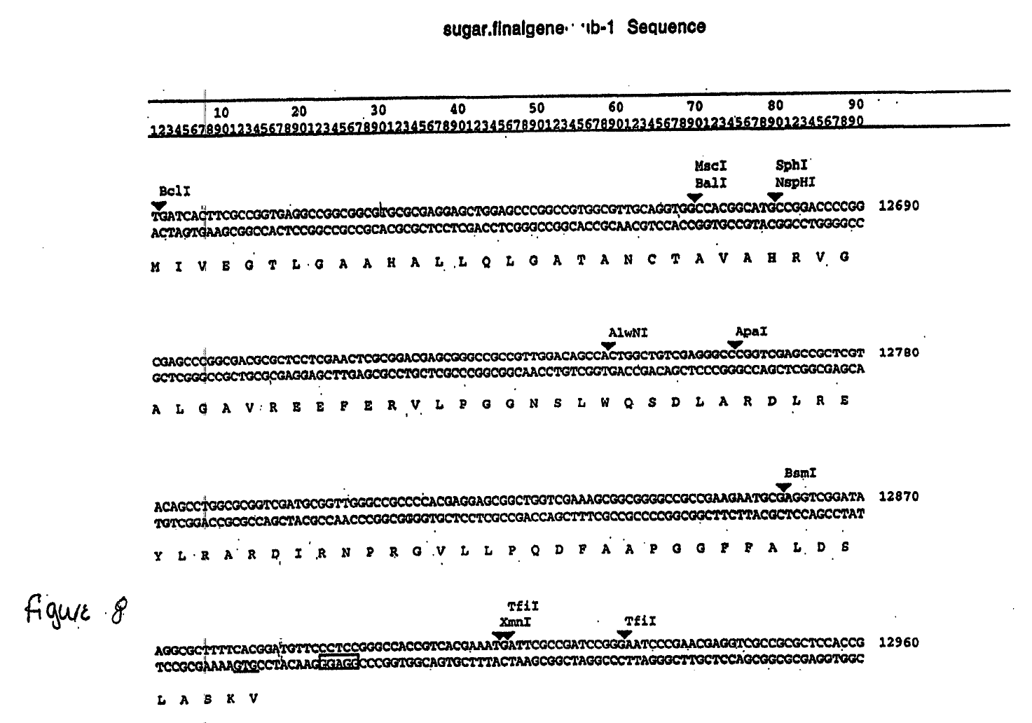

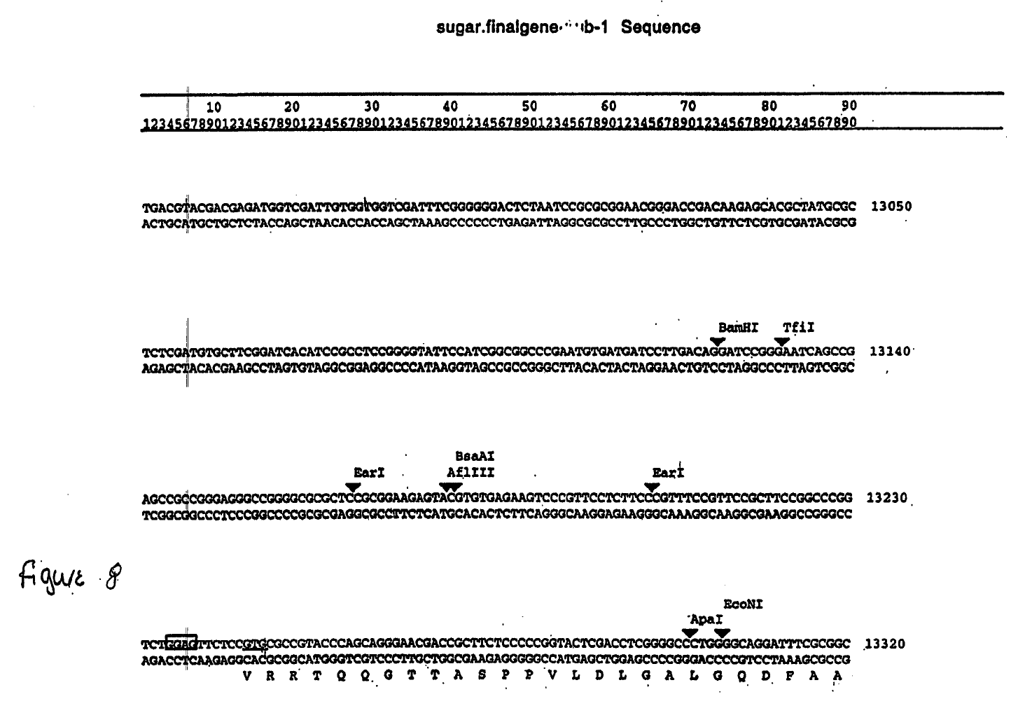

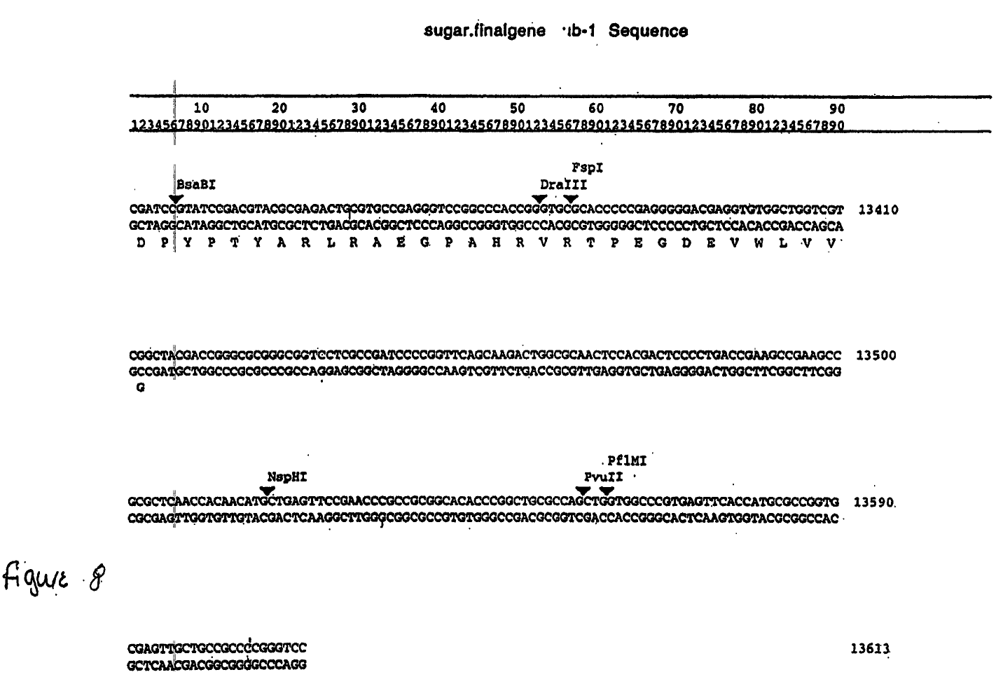

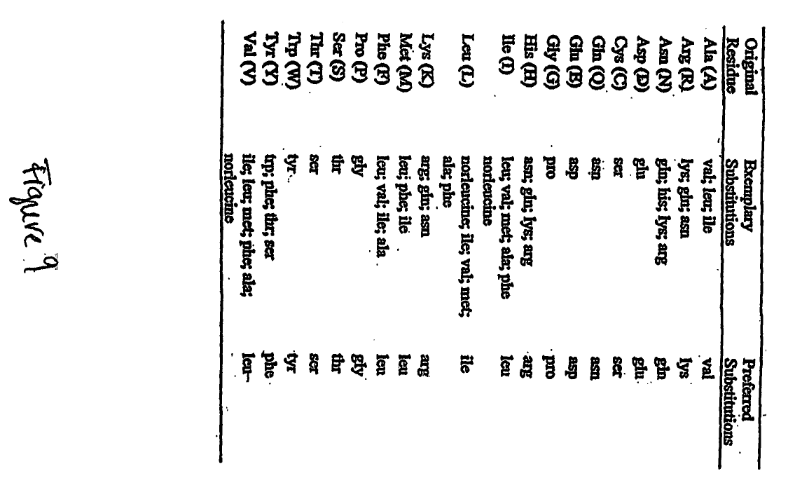

To alter the saccharide structure of a macrolide, the Streptomyces venezuelae met/pik gene cluster was selected as the parent system and a gene from the calicheamicin biosynthetic gene cluster (from Micromonospora echinospora spp. Calichensis) as the foreign gene. The parent cluster encodes the biosynthetic enzymes for methymycin, neomethymycin, pikromycin, and narbomycin, of which all are macrolides containing desosamine as the sole sugar component for antibiotic activity (Xue et al., 1998; Zhao et al., 1998) Eight open reading frames (desI-desVIII) in this cluster have been assigned as genes involved in desosamine biosynthesis (FIG. 15). The antitumor agent calicheamicin (26) contains four unique sugars crucial to tight DNA binding (Kₐ about 10⁶-10⁸), one of which (29) is derived from 4-amino-4,6-dideoxyglucose (28) and is part of the unusually restricted N—O connection between sugars A and B (FIG. 16) (Ding et al., 1991; Drak et al., 1991; Walker et al., 1991; Ellestad et al.; Borders et al., 1995). Compound 28 is anticipated to be derived from the corresponding 4-ketosugar 27 via a transamination reaction, and recent work has led to the assignment of a gene (calH) as encoding a C-4 aminotransferase (FIG. 16) (Alhert et al.). Interestingly, the proposed substrate for CalH, 27, is also an intermediate in the desosamine pathway and is expected to exist in a tautomerase (DesIII)-mediated equilibrium with the substrate for DesI (Chen et al., 1999). Thus, it is conceivable that 27 might accumulate in a desI or desVIII disruption/deletion S. venezuelae mutant strain. Heterologous expression of calH in this mutant may reconstitute a hybrid pathway towards new methymycin/pikromycin derivatives which carry the 4-amino-4,6-dideoxy glucose derived from 26.

To test this, the 1.2 kb calH gene was amplified by polymerase chain reaction (PCR) from pJST1192_{Kpn7.0Kb}, a subclone containing a 7.0 kb KpnI fragment of cosmid 13a (Thorson et al., 1999). The amplified gene was cloned into the EcoRI/XbaI sites of the expression vector pDHS617, which contains an apramycin resistance marker. pDHS617 is derived from pOJ446 (Bierman et al., 1992), and a promoter sequence from met/pik (Xue et al., 1998). The resulting plasmid, pLZ-C242, was introduced by conjugal transfer using Escherichia coli S 17-1 (Bierman et al., 1992) into a previously constructed S. venezuelae mutant (Kdes1) (Borisova et al., 1999) in which desI was replaced by the neomycin resistance gene that also confers resistance to kanamycin. The pLZ-C242 containing S. venezuelae-KdesI colonies were identified on the basis of their resistance to apramycin antibiotic (Apr^{R}). One of the engineered strains, KdesI/calH-1, was first grown in 100 mL of seed medium at 29° C. for 48 hours and then inoculated and grown in vegetative medium (5 L) for another 48 hours (Cane et al., 1993). The fermentation broth was centrifuged to remove the cellular debris and mycelia, and the supernatant was adjusted to pH 9.5 with concentrated KOH followed by chloroform extraction. The crude products (700 mg) were subjected to flash chromatography on silica gel using a gradient of 0-20% methanol in chloroform. A major product, 10-deoxymethynolide, and a mixture of two minor macrolide compounds were obtained. The two macrolides were further purified by HPLC on a C₁₈ column using an isocratic mobile phase of acetonitrile/H₂O (1:1). They were later identified as 31 (11.0 mg) and 32 (1.5 mg) by spectral analyses. The spectral data of 31 is: ¹H NMR (500 MHz, CDCl₃) δ 6.75 (1H, dd, J=16.0, 5.5, 9-H), 6.44 (1H, dd, J=16.0, 1.2, 8-H), 5.34 (1H, d, J=8.0, N—H), 4.96 (1H, m, 11-H), 4.27 (1H, d, J=7.5, 1′-H), 3.66 (1H, dd, J=9.5, 8.0, 4′-H), 3.60 (1H, d, J=10.5, 3-H), 3.50 (1H, t, J=9.5, 3′-H), 3.4 (1H, m, 5′-H), 3.4 (1H, m, 2′-H), 2.84 (1H, dq, J=10.5, 7.5, 2-H), 2.64 (1H, m, 10-H), 2.53 (1H, m, 6-H), 2.06 (3H, s, Me—C═O), 1.7 (1H, m, 12-H), 1.66 (1H, m, 5-H), 1.56 (1H, m, 12-H), 1.4 (1H, m, 5-H), 1.36 (3H, d, J=7.5, 2-Me), 1.25 (3H, d, J=6.5, 5′-Me), 1.24 (1H, m, 4-H), 1.21 (3H, d, J=7.5, 6-Me), 1.10 (3H, d, J=6.5, 10-Me), 0.99 (3H, d, J=6.0, 4-Me), 0.91 (3H, t, J=7.2, 12-Me). ¹³C NMR (125 MHz, CDCl₃) δ 205.3 (C-7), 175.1 (C-1), 171.9 (Me—C═O), 147.1 (C-9), 126.1 (C-8), 103.0 (C-1′), 85.8 (C-3), 75.8 (C-5′), 75.8 (C-3′), 74.1 (C-11), 70.8 (C-2′), 57.6 (C-4′), 45.3 (C-6), 44.0 (C-2), 38.1 (C-10), 34.2 (C-5), 33.6 (C-4), 25.4 (C-12), 23.7 (Me—C═O), 18.1 (C-6′), 17.9 (6-Me), 17.6 (4-Me), 16.4 (2-Me), 10.5 (12-Me), 9.8 (10-Me). High-resolution FAB-MS calcd for C₂₅H₄₂NO₈ (M+H⁺) 484.2910, found 484.2903. The spectral data of 32 is: ¹H NMR (500 MHz, CDCl₃) δ 6.69 (1H, dd, J=16.0, 6.0, 11-H), 6.09 (1H, dd, J=16.0, 1.5, 10-H), 5.35 (1H, d, J=8.5, N—H), 4.96 (1H, m, 13-H), 4.36 (1H, d, J=7.5, 1′-H), 4.19 (1H, m, 5-H), 3.83 (1H, q, J=6.5, 2-H), 3.68 (1H, dt, J=10.0, 8.5, 4′-H), 3.52 (1H, t, J=8.5, 3′-H), 3.50 (1H, m, 5′-H), 3.42 (1H, t, J=7.5, 2′-H), 2.92 (1H, dq, J=7.0, 5.0, 4-H), 2.81 (1H, m, 8-H), 2.73 (1H, m, 12-H), 2.06 (3H, s, Me—C═O), 1.8 (1H, m, 6-H), 1.6 (1H, m, 14-H), 1.55 (1H, m, 7-H), 1.37 (3H, d, J=6.5, 2-Me), 1.32 (3H, d, J=7.0, 4-Me), 1.3 (1H, m, H-14), 1.27 (3H, d, J=6.5, 5′-Me), 1.25 (1H, m, 7-H), 1.12 (3H, d, J=6.0, 8-Me), 1.11 (3H, d, J=6.5, 12-Me), 1.07 (3H, d, J=6.0, 6-Me), 0.91 (3H, t, J=7.2, 14-Me). High-resolution FAB-MS calcd for C₂₈H₄₆NO₉ (M+H⁺) 540.3172, found 540.3203.

The observed production of macrolides 31 and 32 by the KdesI/calH-1 has vast implications. First, the appended hexose (33), which indeed carries the predicted amino group at C-4, provides indisputable support for the calH gene assignment as encoding the TDP-6-deoxy-D-glycero-L-threo-4-hexulose 4-aminotransferase of the calicheamicin pathway. Second, the successful expression of the CalH protein in S. venezuelae by the newly constructed expression vector highlights the potential of using this system to express other foreign genes in this strain, a prerequisite for developing more elaborate combinatorial biosynthetic strategies. Moreover, this result also reveals that the glycosyltransferase (DesVII) of this pathway can recognize alternative sugar substrates (such as 28) whose structures are considerably different from the original amino sugar substrate, TDP-D-desosamine. While the sugar component in the products is expected to be the aminodeoxy hexose 28, the 4-amino group of the attached sugar component in 31 and 32 is N-acetylated. It is not clear at this point whether the acetylation occurs on the free sugar or after it is appended to the aglycone. Since both 31 and 32 are new compounds synthesized in vivo by the S. venezuelae mutant strain, the observed N-acetylation might be a necessary step for self-protection (Cundliffe, 1989; Cundlife, 1992; McManus, 1997). Indeed, purified 31 and 32 are inactive against Streptococcus pyogenes grown on Mueller-Hinton agar plates (Managahas, 1996), while the controls (methymycin and pikromycin) show clearly visible inhibition zones.

Another noteworthy, albeit unexpected result was the fact that the aglycone of the isolated macrolide 31 was 10-deoxymethynolide instead of methymycin and neomethymycin analogues that are hydroxylated. Interestingly, the aglycone of 32 was the 14-membered narbonolide that is also devoid of hydroxylation. It is possible that the cytochrome P450 hydroxylase (PikC), which catalyzes the hydroxylation of 10-deoxymethynolide and narbonolide (Xue et al., 1998) is sensitive to structural variations on the appended sugar. Indeed, no aglycone hydroxylation was discernible when 31 and 32 were incubated with purified PikC in vitro. A similar observation was also noted in the case where desosamine was replaced by quinovose (Example 4). It could be argued that the presence of a substituent (either hydroxyl or amino group) at C-4 in the sugar moiety is responsible, at least in part, for decreasing or preventing hydroxylation of the macrolide.

In conclusion, the results show that non-natural secondary metabolite glycosylation patterns can be engineered through a rational selection of heterologous gene combinations. This demonstrated ability to engage foreign enzymes in concert with the natural biosynthetic machinery offers a tremendous potential to generate further structural diversity. By extending the present study, the construction of diverse nucleotide sugar glycosylation precursor pools may soon substantially enhance current novel drug discovery through combinatorial biosynthesis efforts.

### EXAMPLE 6

### Engineering a Hybrid Sugar Biosynthetic Pathway

The 6-deoxy-4-hexulose 33 in the desosamine pathway has also been suggested as a biosynthetic intermediate for TDP-L-dihydrostreptose (35), the precursor of streptose (36) found in the antibiotic streptomycin (37) of Streptomyces griseus (FIG. 16) (Ortmann et al., 1974; Wahl et al., 1975; Maier et al. 1975; Wahl et al. 1979). With the tentative assignment of genes in the streptomycin cluster (Pisowotzki et al., 1991; Distler et al. 1992), a biosynthetic pathway for TDP-L-dihydrostreptose has been postulated. As illustrated in FIG. 16, the strM gene may encode a 3,5-epimerase responsible for the conversion of 33 to 34, while the product of strL gene is speculated to catalyze the ring contraction of 34 to give 35 (Pisowotzki et al., 1991; Distler et al. 1992). Since the proposed substrate for StrM, 33, is also an intermediate in the desosamine pathway, heterologous expression of StrM, StrL, or StrM/StrL in the S. venezuelae desI-mutant in which 33 accumulates, may reconstitute hybrid pathways toward new methymycin/pikromycin derivatives carrying an L-pyranose or an L-furanose.

In these experiments, the strM (0.8 kb) and strL (1.0 kb) genes were separately amplified by polymerase chain reaction (PCR) from the genomic DNA of S. griseus. The amplified strM gene was cloned into the EcoRI/NsiI sites of the expression vector pDHS702 (Xue et al., 2000), which contains a thiostrepton resistance marker. The strL gene was cloned into the EcoRI/XbaI sites of the vector pDHS617, which has an apramycin resistance marker. Each plasmid was transformed into Escherichia coli S 17-1 (Bierman et al., 1992) and then introduced separately by conjugal transfer into the previously constructed mutant S. venezuelae KdesI. The resulting strains, KdesI/strM and KdesI/strL, were identified on the basis of their resistance to the corresponding antibiotics. Using the same strategy, the strL-containing plasmid was further engineered into the KdesI/strM mutant to produce the recombinant strain KdesI/strM/strL, which confers resistance to both apramycin and thiostrepton. One such strain, KdesI/strM/strL-8, was chosen to grow in 150 mL of seed medium at 29° C. for 48 hours, and then inoculated and grown in vegetative medium (6 L) for another 48 hours (Cane et al., 1993). The fermentation broth was centrifuged, and the supernatant was extracted with chloroform. After concentration, the residual yellow oil (1.5 g) was subjected to flash chromatography on silica gel using 10% methanol in chloroform as eluent. The crude products were further purified by HPLC on a C₁₈ column eluted with a linear gradient of 0-50% acetonitrile in water over 20 minutes to yield four new macrolide derivatives, 38 (31.1 mg), 39 (6.3 mg), 40 (3.0 mg), and 41 (3.9 mg).

Spectral analysis of these compounds revealed that 38-40 are 12-membered macrolide derivatives, while 41 is a 14-membered macrolide. Spectral data of 38: ¹H NMR (500 MFz, acetone-d₆, J in hertz) 0.86 (3H, t, J=7.5, 12-Me), 1.00 (3H, d, J=6.5, 4-Me), 1.19 (3H, d, J=7.0, 6-Me), 1.20 (3H, d, J=5.0, 5′-Me), 1.18-1.30 (1H, m, 4-H), 1.28 (3H, d, J=6.5, 2-Me), 1.27-1.37 (1H, m, 5-H), 1.36 (3H, s, 10-Me), 1.50 (1H, ddq, J=10.8, 14.3, 7.1, 12-H), 1.83 (1H, t, J=13.5, 5-H), 1.99 (1H, ddq, J=2.0, 14.0, 7.3, 12-H), 2.44-2.54 (1H, m, 6-H), 2.84 (1H, dq, J=10.0, 6.8, 2-H), 3.42 (1H, t, J=9.3, 4′-H), 3.49 (1H, d, J=10.5, 3-H), 3.58 (1H, dd, J=3.5, 9.5, 3′-H), 3.70 (1H, dq, J=9.3, 6.2, 5′-H), 3.94 (1H, brs, 2′-H), 4.70 (1H, dd, J=2.0, 10.5, 11-H), 4.72 (1H, brs, 1′-H), 6.53 (1H, d, J =16.0, 8-H), 6.57 (1H, d, J=16.0, 9-H); ¹³C NMR (126 MHz, acetone-d₆) 11.2 (C-13), 16.8 (2-Me), 17.4 (6-Me), 17.8 (4-Me), 17.9 (C-6′), 19.3 (10-Me), 21.7 (C-12), 34.2 (C-4), 34.4 (C-5), 44.8 (C-2), 46.2 (C-6), 70.0 (C-5′), 72.1 (C-2′), 72.5 (C-3′), 73.4 (C-4′), 74.4 (C-10), 77.2 (C-11), 89.0 (C-3), 104.4 (C-1′), 125.7 (C-8), 150.8 (C-9), 175.4 (C-1), 203.6 (C-7); high-resolution FAB-MS calcd for C₂₃H₃₈O₉Na (M+Na)⁺ 481.2414, found 481.2444. Spectral data of 39: ¹H NMR (500 MHz, acetone-d₆, J in hertz) 1.00 (3H, d, J=6.5, 4-Me), 1.12 (3H, d, J=6.5, 12-Me), 1.17 (3H, d, J=7.0, 10-Me), 1.199 (3H, d, J=7.5, 6-Me), 1.200 (3H, d, J=6.0, 5′-Me), 1.22-1.33 (1H, m, 4-H), 1.25 (3H, d, J=7.0, 2-Me), 1.27-1.39 (1H, m, 5-H), 1.78 (1H, t, J=13.3, 5-H 2.42-2.51 (1H, m, 6-H), 2.77-2.85 (1H, m, 2-H), 3.06-3.13 (1H, m, 10-H), 3.42 (1H, t, J=9.5, 4′-H), 3.48 (1H, d, J=10.5, 3-H), 3.58 (1H, brd, J=9.0, 3 ′-H), 3.70 (1H, dq, J=9.4, 6.3, 5′-H), 3.79-3.87 (1H, m, 12-H), 3.93 (1H, brs, 2′-H), 4.71 (1H, s, 1′-H), 4.75 (1H, dd, J=2.0, 9.5, 11-H), 6.59 (1H, d, J=15.5, 8-H), 6.69 (1H, dd, J=5.3, 15.8, 9-H); ¹³C NMR (126 MHz, acetone-d₆) 9.9 (10-Me), 16.4 (2-Me), 17.5 (6-Me), 17.8 (4-Me), 17.9 (C-6′), 21.4 (13-Me), 34.2 (C-4), 34.5 (C-5), 36.3 (C-10), 44.5 (C-2), 46.1 (C-6), 66.1 (C-12), 70.0 (C-5′), 72.2 (C-2′), 72.5 (C-3′), 73.4 (C-4′), 76.8 (C-11), 89.1 (C-3), 104.4 (C-1′), 126.7 (C-8), 148.2 (C-9), 175.0 (C-1), 203.8 (C-7); high-resolution FAB-MS calcd for C₂₃H₃₈O₉Na (M+Na)⁺ 481.2414, found 481.2426. Spectral data of 40: ¹H NMR (500 Mz, acetone-d₆, J in hertz) 0.89 (3H, t, J=7.3, 12-Me), 1.00 (3H, d, J=7.0, 4-Me), 1.13 (3H, d, J=7.0, 10-Me), 1.196 (3H, d, J=7.0, 6-Me), 1.197 (3H, d, J=6.0, 5′-Me), 1.17-1.27 (1H, m, 4-H), 1.25 (3H, d, J=7.0, 2-Me), 1.30-1.37 (1H, m, 5-H), 1.57-1.73 (2H, m, 12-Hs), 1.79 (1H, t, J=12.8, 5-H), 2.42-2.50 (1H, m, 6-H), 2.69-2.74 (1H, m, 10-H), 2.75-2.83 (1H, m, 2-H), 3.41 (1H, t, J=9.3, 4′-H), 3.47 (1H, d, J=10.0, 3-H), 3.57 (1h, dd, J=3.0, 9.5, 3′-H), 3.70 (1H, dq, J=9.3, 6.3, 5′-H), 3.94 (1H, brs, 2′-H), 4.71 (1H, brs, 1′-H), 4.96 (1H, ddd, J=2.3, 5.3, 9.3, 11-H), 6.58 (1H, dd, J=1.3, 16.3, 8-H), 6.70 (1H, dd, J=5.3, 16.3, 9-H); ¹³C NMR (126 MHz, acetone-d₆) 9.8 (10-Me), 10.8 (C-13), 16.7 (2-Me), 17.5 (6-Me), 17.8 (4-Me), 17.9 (C-6′), 25.9 (C-12), 34.3 (C-4), 34.5 (C-5), 38.9 (C-10), 44.7 (C-2), 46.0 (C-6), 70.0 (C-5′), 72.2 (C-2′), 72.5 (C-3′), 73.5 (C-4′), 74.6 (C-11), 89.2 (C-3), 104.4 (C-1′), 126.5 (C-8), 147.8 (C-9), 175.4 (C-1), 203.9 (C-7); high resolution FAB-MS calcd for C₂₃H₃₉O₈(M+H)⁺ 443.2645, found 443.2620. Spectral data of 41: ¹H NMR (500 MHz, acetone-d₆, J in hertz) 0.89 (3H, t, J=7.3, 16-Me), 1.01 (3H, d, J=7.0, 6-Me), 1.11 (3H, d, J=6.0, 12-Me), 1.13 (3H, d, J=7.0, 8-Me), 1.16-1.19 (1H, m, 7-H), 1.22 (3H, d, J =6.5, 5′-Me), 1.277 (3H, d, J=7.0, 4-Me), 1.284 (3H, d, J=7.0, 2-Me), 1.49-1.74 (4H, m, 6-H, 7-H, 14-Hs), 2.69-2.78 (2H, m, 8-H, 12-H), 2.97-3.04 (1H, m, 4-H), 3.41 (1H, t, J=9.5, 4′-H), 3.60 (1H, dd, J=3.3, 9.3, 3′-H), 3.70 (1H, dq, J=9.4, 6.3, 5′-H), 3.85 (1H, brs, 2′-H), 3.95 (1H, brs, 5-H), 4.08 (1H, q, J =7.0, 2-H), 4.80 (1H, brs, 1′-H), 4.91 (1H, dt, J =9.3, 3.3, 13-H), 6.16 (1H, brd, J=15.5, 10-H), 6.69 (1H, dd, J=5.0, 15.5, 11-H); high-resolution FAB-MS calcd for C₂₆H₄₃O₉ (M+H)⁺ 499.2907, found 499.2924.

Interestingly, the stereochemistry of the linkage between the aglycone and the appended sugar in 38-41 was established to be (J_{1′2′}=0 Hz), which is distinct from the glycosidic linkage (d, J_{1′2′}=6.5-7.5 Hz) found in the wild type structures. While these new compounds all carry an identical 6-deoxyhexose, the NMR data could not distinguish whether the appended sugar is L-rhamnose (42) or its enantiomer, 6-deoxy-D-mannose (43). In order to unambiguously identify the newly incorporated sugar, 38 was treated with dimethoxypropane followed by derivatization with (S) or (R)-MTAP chloride to generate the corresponding Mosher's esters (44 and 45). Since the orientation of the phenyl ring of MTAP is different in these two diastereomers, the protons adjacent to MTAP will experience differential shielding depending on their spatial relationship with respect to the anisotropic cone of the aryl group (Ohtani et al., 1991; Ferreiro et al., 1991). On the basis of this well-documented phenomenon, the absolute stereochemistry of the chiral center (C-4′) can be deduced from the difference in the chemical shifts measured as =_{(S)-MTPA ester}−_{(R)-MTPA ester}. As shown in the bottom of FIG. 16, positive values were observed for 1′-H, 2′-H, 3′-H, 4′-H, and the two methyl signals of the acetonide group, while negative values were recorded for 5′-H and 5′-Me. These finding are indicative of an S configuration at C-4′, allowing the attached sugar in 3841 to be assigned as -L-rhamnose.

With the identification of L-rhamnose (42) as the sugar component of metabolites 38-41 produced by the engineered KdesI/strM/strL strain, the role of StrM as a 3,5-epimerase converting 33 to 34 is clearly confirmed. The corresponding methymycin/pikromycin derivatives carrying a -linked D-quinovose (47, 6-deoxy-D-glucose) were produced by the KdesI/strL strain (FIG. 16). These quinovose-containing macrolides were also found as metabolites of the host strain, S. venezuelae KdesI and the KdesI/strM strain. Since the substrate of StrL is expected to be 34, in the absence of StrM to catalyze the necessary D-/L-conversion of 33 to provide 34, it is not surprising that both KdesI/strL and KdesI strains produce the same macrolide compounds as observed. The fact that no new macrolide products were found in the broth of the KdesI/strM strain may be attributed to the instability of 34 in vivo, or the inability of the glycosyltransferase DesVII to process 34 as a substrate. Apparently, the host strain of S. venezuelae KdesI contains a pathway-independent D-hexulose reductase that can reduce 33 to TDP-D-quinovose (46), but lacks an L-hexulose reductase of its own to reduce 34. The StrM catalyzed epimerization is expected to be reversible. Thus, in the presence of a D-hexulose reductase, the equilibrium between 33 and 34 in the KdesI/strM strain will be shifted toward 33, which after reduction gives quinovose as observed in the product. Since L-rhamnose is formed only in the strL-containing strain, one can conclude that, in addition to its putative function as dihydrostreptose synthase, StrL could also serve as a sugar reductase capable of reducing an L-6-deoxy-4-hexulose such as 34 to TDP-L-rhamnose (48).

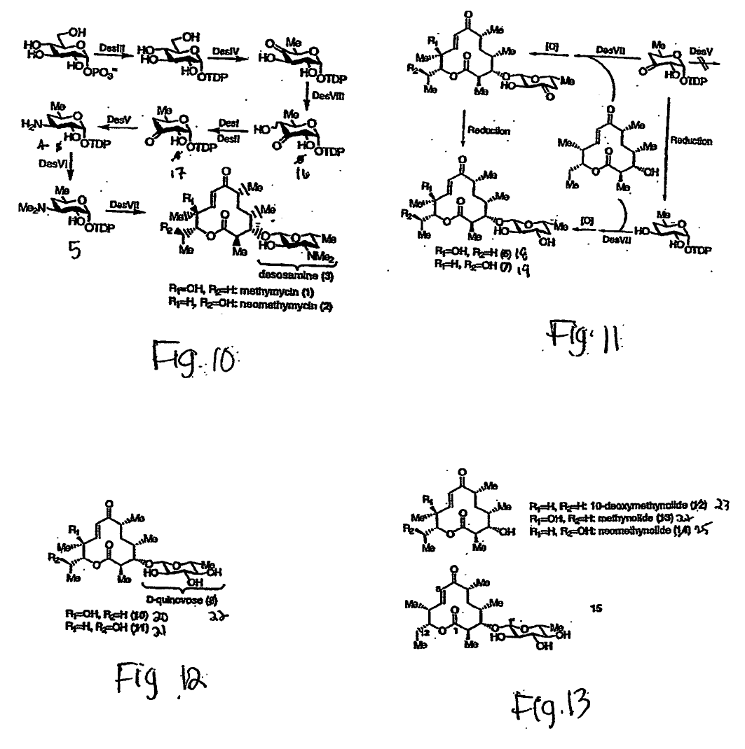

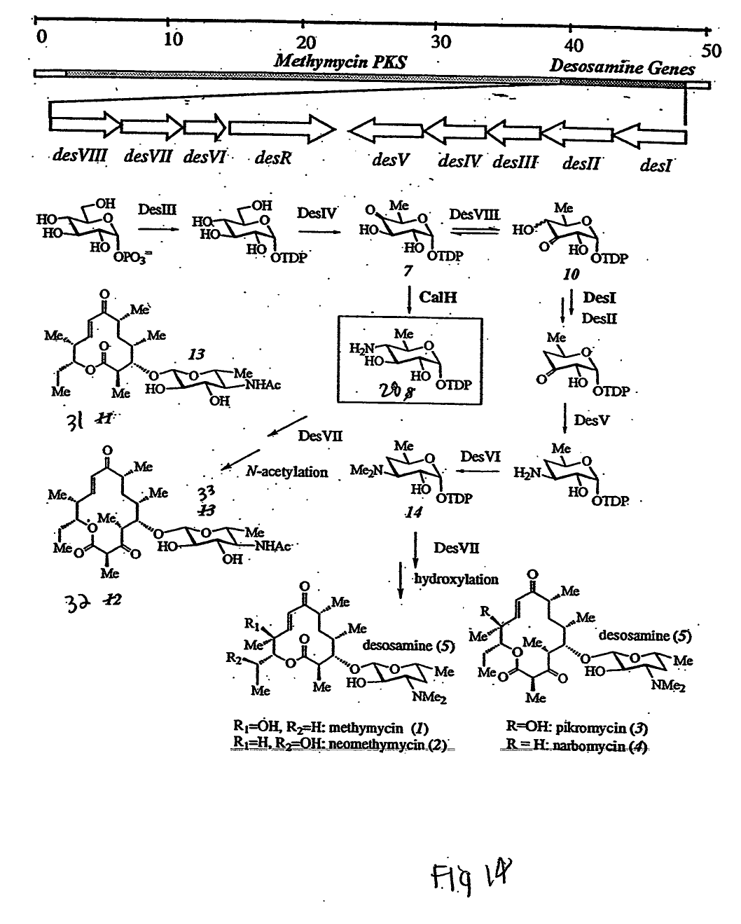

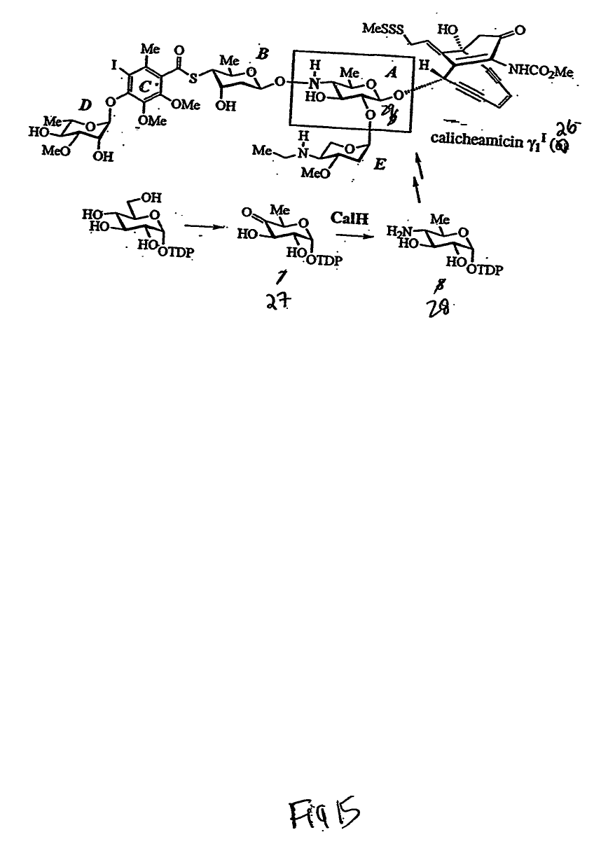

It should be noted that the mechanism of the ring contraction step in the dihydrostreptose pathway is remarkably similar to that proposed for the biosynthesis of UDP-D-apiose (50), which is derived from UDP-D-glucuronic acid (49) catalyzed by apiose synthase (FIG. 18) (Kelleher et al., 1972; Gebb et al., 1975; Watson et al., 1975; Matem et al., 1977; Wahl et al., 1978). This synthase has been assigned to have dual functions, possessing both 4-hexulose reductase and ring-contraction activities, since UDP-D-xylose (51) is a byproduct of the catalysis of apiose synthase (Kelleher et al., 1972; Gebb et al., 1975; Watson et al., 1975; Matem et al., 1977; Wahl et al., 1978). Thus, the fact that StrL resembles apiose synthase having hexulose reductase activity lends strong credence for an analogous role of StrL as the catalyst for the ring contraction step in the dihydrostreptose pathway. The failure to detect the incorporation of 35 into the macrolide structures may simply reflect the limitation of DesVII to accommodate a furanose in its active site.

The results described here present a rare example of a glycosyltransferase that recognizes both D- and L-sugar as substrates (Wohlert et al., 1998). The established versatility of this glycosyltransferase (DesVH) on substrate selection highlights its potential as a catalyst in the construction of new macrolides carrying a broad range of modified sugars, a prerequisite for developing more exquisite combinatorial biosynthetic strategies for new antibiotics. This work once again demonstrates the feasibility of engineering secondary metabolite glycosylation through a rational selection of gene combinations.

### EXAMPLE 7

### Synthesis of TDP-4-amino-4,6 dideoxy-D-glucose by DesII

Carbohydrates are the focus of growing attention among biological molecules in recent years due to the increased appreciation of their vital roles in many physiological processes (Weymouth-Wilson et al., 1997). As components of many glycoconjugates, sugars, particularly the deoxysugars, contribute to a diverse repertoire of biological activities. Since modifying the structure of the appended sugars holds promise for varying or enhancing the biological activities of the parent glycoconjugates, there is considerable and continuing effort to explore how these unusual sugars are made in the producing organisms (Hallis et al., 1999; He et al., 2000). Such striving has led to the discovery of several elegant strategies evolved in nature for breaking the C—O bond of a hexose sugar. Thus, presently it can be concluded that a sequence of α,β-dehydration followed by a hydride reduction is the mechanism for β-deoxygenation of a ketosugar precursor (Draeger et al., 1999; Chen et al., 1999) whereas a collaborative catalysis by a pyridoxamnine 5′-phosphate (PMP)-dependent [2Fe-2S]-containing enzyme (E₁) and an NADH-dependent iron-sulfur flavoprotein reductase (E₃) is require for α-deoxygenation of a ketosugar substrate (Thorson et al., 1993; Johnson et al., 1996; Chang et al., 2000).

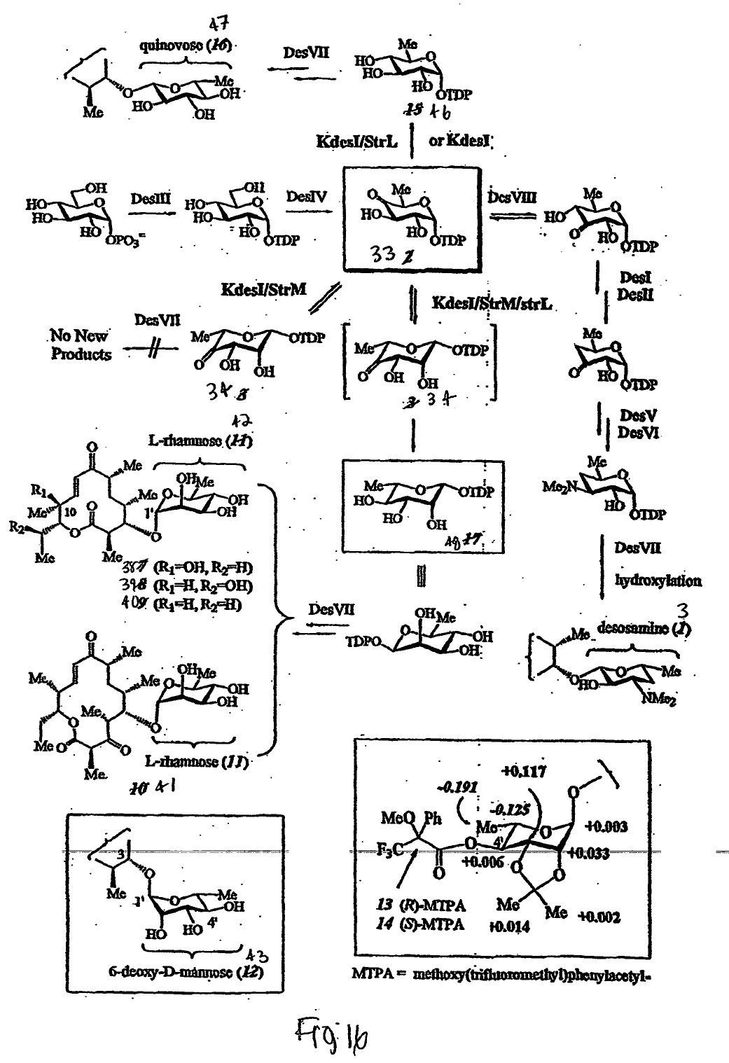

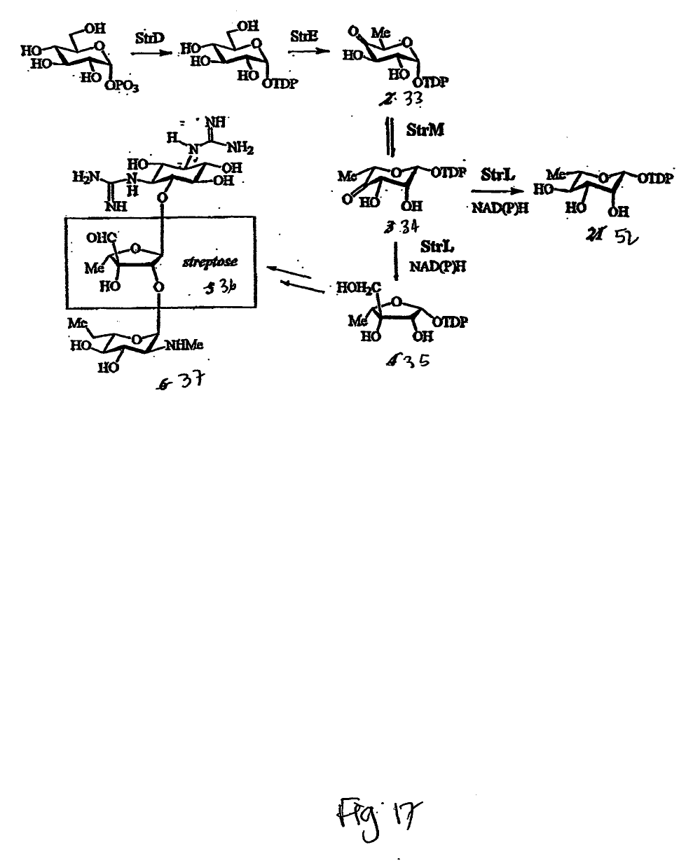

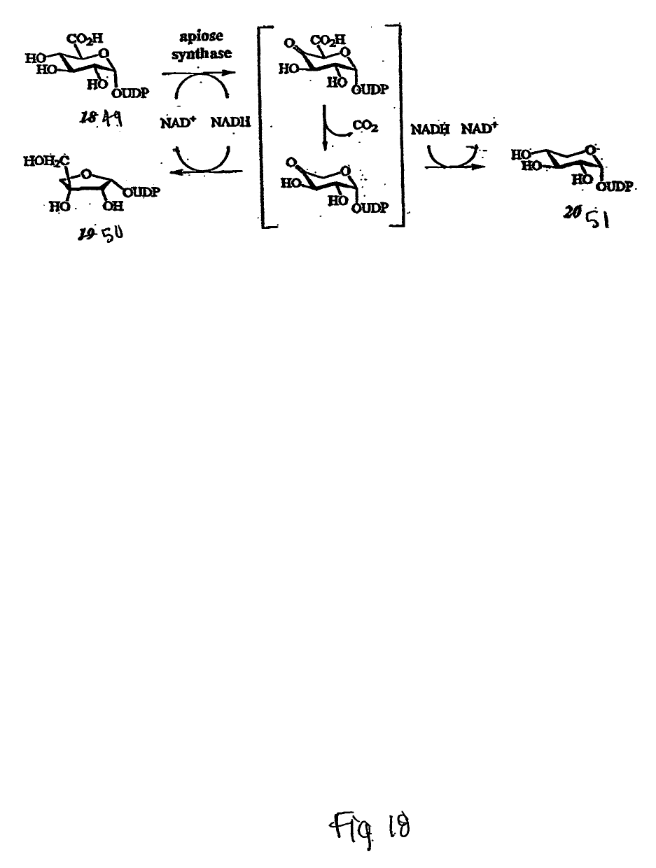

While the mechanisms of C—O bond cleavage at C-2, C-3, and C-6 of a hexose have been fully established (Hallis et al., 1999; He et al., 2000), little is known about the mode of C—O bond scission at C-4 in making 4-deoxygenated sugars. Genetic studies on the biosynthesis of D-desosamine (1 in FIG. 19), a 3-(dimethylamino)-3,4,6-trideoxyhexose found in a number of antibiotics, resulted in the identification of the entire desosamine biosynthetic gene cluster from Streptomyces venezuelae (Scheme 1, FIG. 19) (Zhao et al., 1998; Xue et al., 1998), which produces methymycin (2), neomethymycin (3), pikromycin (4), and narbomycin (5). From this, eight open reading frames (desI-desVIII) within this cluster are suggested to be involved in desosamine biosynthesis including desI and desII that are assigned to be associated with the C-4 deoxygenation step (Zhao et al., 1998; and see Gaisser et al., 1997; Summers et al., 1997, which relate to the DesI/DesII equivalents in the erythromycin gene cluster, i.e, EryCIV/EryCV). Since the translated sequence of desI shows high homology to B₆-dependent enzymes and is 24% identical to that of E₁, and the translated desII sequence contains a conserved motif of CXXXCXXC (SEQ ID NO:50) characteristic for a [4Fe-4S] center (Ruzicka et al., 2000), the C-4 deoxygenation has been postulated to follow a path similar to that catalyzed by E₁ and E₃ (Zhao et al., 1998; and see Gaisser et al., 1997; Summers et al., 1997). As illustrated in Scheme 1 (FIG. 19), the reaction may be initiated by a tautomerization step presumably catalyzed by DesVIII to convert 6, a common precursor for 6-deoxyhexoses, to 3-keto-6-deoxyhexose 7. DesI and DesII may then effect the removal of 4-OH from 7 to give the 3-keto-4,6-dideoxyhexose product (8) which has earlier been confirmed as the substrate of the next enzyme in the pathway, DesV (Zhao et al., 1998). This proposal is supported by the fact that 4-OH is retained in the appended sugar (D-quinovose, 9) of the modified methymycin and pikromycin derivatives produced by the desI deleted mutant (Borisova et al., 1999). To learn more about this C—O bond cleavage event, targeted disruption of the desII gene and functional analyses of the DesI enzyme were conducted.

To confirm whether DesII is a part of the C-4 deoxygenation machinery, a S. venezuelae mutant was generated in which the desII gene was replaced by the kanamycin resistance gene through homologous recombination of a plasmid containing the appropriate insert with the wild-type S. venezuelae chromosome (Bierman et al., 1992). This mutant strain was isolated and used for fermentation as previously described (Zhao et al., 1998; Cnae et al., 1993). It should be pointed out that E₁-catalyzed dehydration is a reversible reaction with equilibrium favoring the reverse direction (Weigel et al., 1992), and the reduction by E₃ is essential to drive the overall reaction to completion. Hence, if C-4 deoxygenation follows a path similar to E₁/E₃ catalysis and DesII is an E₃-equivalent, disruption of the desII gene is expected to give a mutant with a phenotype that is identical to the desI mutant. Indeed, no wild-type antibiotics were found in the fermentation broth (6 L) of desII deleted mutant; instead, two macrolides containing an N-acetylated 4-aminosugar, 11 (2.4 mg) and 12 (1 mg), were obtained (see Zhao et al., 1998). Compounds 11 and 12 are likely derived from the coupling of 10 and the respective aglycons, followed by N-acetylation (Scheme 1). However, it is also possible that N-acetylation of 10 occurs prior to its coupling to the aglycons. Regardless of the sequence of the events, the production of 11 and 12 clearly indicates that 10 must be accumulated in the desII-deleted mutant of S. venezuelae.

The above results provide a hint that DesI is a 4-aminotransferase, and 4-amination is the initial step of 4-deoxygenation. To verify the catalytic function of DesI, the desI gene was amplified by PCR and cloned into the pET-28b(+) expression vector (Novagen) with a His₆-tag at the N-terminus. The produced DesI protein was purified to near homogeneity by a Ni-NTA column (Qiagen) followed by FPLC on a MonoQ column. As judged by SDS-PAGE, the subunit Mᵣ of DesI was estimated to be 45 kDa, which agrees well with the calculated molecular mass of 45 765 Da (plus the His₆ tag). Further analysis by size exclusion chromatography revealed a Mᵣ of 95.6 kDa for DesI. Therefore, DesI exists as a homodimer in solution. The UV-vis spectrum of purified DesI is transparent above 300 nm; however, that of the more concentrated sample shows the presence of trace amount of PLP.

Interestingly, when the putative substrate, TDP-3-keto-deoxy-D-glucose (7) was incubated with the purified DesI in the presence of L-glutamate, no consumption of 7 and no new product were discemable by HPLC analysis. On the contrary, when TDP-4-amino-4-keto-6-deoxy-D-glucose (6) was incubated with DesI under identical conditions, consumption of 6 (retention time=4.52 min) and the formation of a new product (retention=3.87 min) were observed. This new compound was purified by FPLC on a MonoQ column and characterized as the TDP-4-amino-4,6-dideoxy-D-glucose (10). These results firmly establish that DesI only recognizes 4-hexulose 6 as the substrate and will not processes 3-hexulose 7. These findings corroborate well with the desII gene disruption results. As a PLP-dependent 4-aminotransferase, a κ_{cat} value of 56.2±3.1 min⁻¹ and a K_{M} value of 130±4 μM for the sugar substrate 6 were also determined for DesI.

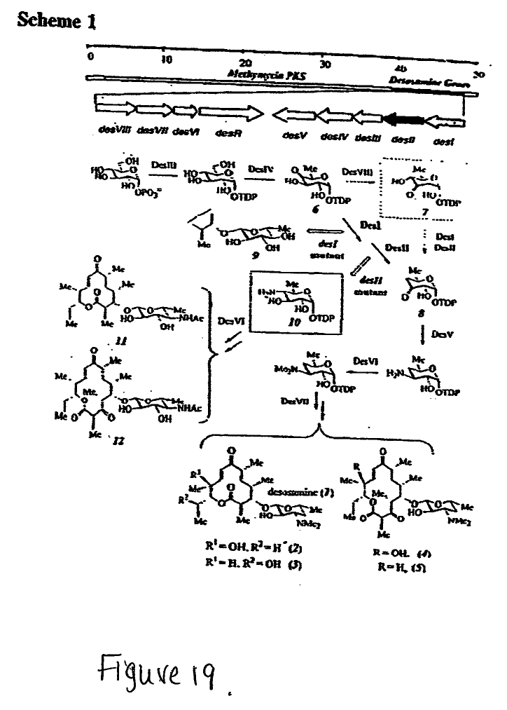

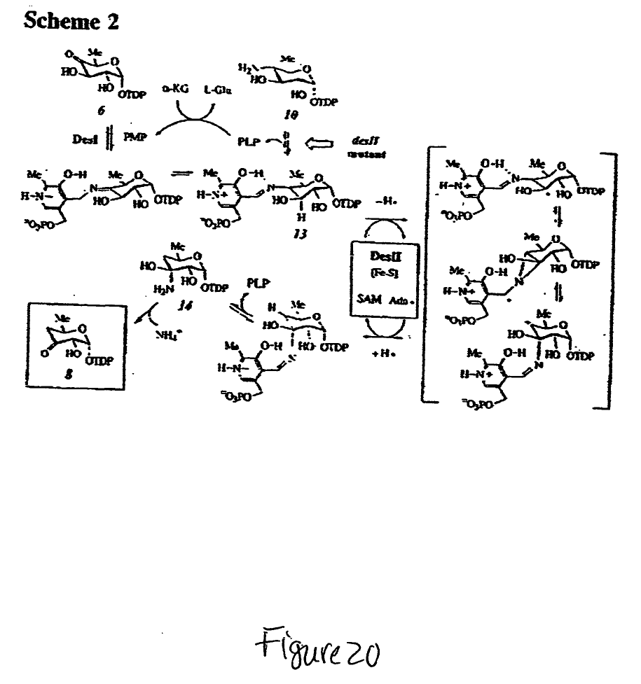

The fact that DesI, in the absence of DesII, catalyzes a transamination reaction on 6 to generate a 4-aminosugar product 10 calls for the modification of the previously proposed biosynthetic pathway for TDP-D-desosamine (Scheme 1, FIG. 19). Clearly, the tautomerization of 6 to 7 is no longer a necessary step in the desosamine pathway. Furthermore, this implies that the mechanism of C-4 deoxygenation cannot be similar to that of the C-3 deoxygenation catalyzed by E₁/E₃ (Hallis et al., 1999; He et al., 2000;Thorson et al., 1993; Johnson et al., 1996; Chang et al., 2000). Considering that DesI/DesII catalysis is initiated by the incorporation of a nitrogen functional group at C-4 (such as 13), a 1,2-nitrogen shift from C-4 to C-3 to generate an aminal intermediate (such as 14) may be the key step of C-4 deoxygenation. As illustrated in Scheme 2, elimination of either a water or an ammonium molecule from C-3 of 14 will generate the 3-keto-4,6-dideoxysugar product (8). There are enzymes capable of promoting 1,2-amino shift. The two best studied examples are ethanolamine ammonia lyase, and adenosylcobalamin (AdoCbl)-dependent enzyme that catalyzes the degradation of ethanolamine to ammonia and acetaldehyde (Babior et al., 1982; LoBrutto et al., 2001; Frey et al., 2000), and lysine 2,3-aminomutase which catalyzes the interconversion of L-lysine and L-⊖-lysine via 1,2-migration of the amino group (Babior et al., 1982; LoBrutto et al., 2001; Frey et al., 2000). The latter enzyme from Clostridium subterminale SB4 contains an iron-sulfur center and is PLP-as well as S-adenosylmethionine (SAM)-dependent. Both reactions are believed to involve a putative 5′-deoxyadenosyl radical which is generated by a reductive cleavage of SAM in lysine 2,3-aminomutase, or a homolytic cleavage of the Co—C bond of adenosylcob(III)alamin in ethanolamine ammonia lyase. This adenosyl radical then abstracts a hydrogen atom from the substrate to initiate the isomerization. Since DesI is a PLP enzyme and DesII has recently been identified as a member of radical SAM superfamily by sequence analyses (Sofia et al., 2001), the DesI and DesII enzymes may work together to catalyze a 1,2-amino migration analogous to that of lysine 2,3-aminomutase (see Scheme 2, FIG. 20) to achieve C-4 deoxygenation. (It is also possible that DesII may act alone by abstracting a 3-H• directly from 10 to generate a radical intermediate which, after deprotonation of OH, is converted to a ketyl equivalent. Subsequence βelimination of 4-amino group followerd by a H^{.} return and tautomerization can also afford 8).

There is no doubt that this study has furnished compelling evidence indicating a new pathway for the biosynthesis of desosamine. These results also allow the postulation of a new mechanism for C-4 deoxygenation,. A comparison of this new mechanism with that of C-3 deoxygenation clearly shows that nature has evolved diverse and elaborate strategies to pursue the removal of an α-OH from a ketohexose precursor in the biosynthesis unusual sugars. Taken together, studies conducted on the biosynthesis of deoxyhexoses have infused refreshing mechanistic insights into the general routes of biological deoxygenations. These findings are a good testament to the evolutionary diversity of biological C—O bond cleavage events (Johnson et al., 1999).

References

Andersen, J. R., Hutchinson, C. R. J. Bacteriol., 174:725-735 (1992).

Aparicio, J. F., Molnar, I., Schwecke, T., Konig, A., Haydock, S. F., Khaw, L. E., Staunton, J., Leadlay, P. F. Gene, 169:9-16 (1996).

Arisawa, A., Kawamura, N., Takeda, K., Tsunekawa, H., Okamura, K., Okamoto, R. Appl. Environ. Microbiol., 60:2657-2660 (1994).

August, P. R., Tang, L., Yoon, Y. J., Ning, S., Muller, R., Yu, T. W., Taylor, M., Hoffmann, D., Kim, C. G., Zhang, X., Hutchinson, C. R. & Floss, H. G. Chem. Biol., 5:69-79 (1998).

Babior, B. M., In B₁₂ Dolphin, D., Ed.; Wiley: New York, 1982; Vol. 2, Chapter 10.

Baltz, R. H., Seno, E. T. Annu. Rev. Microbiol., 42:547-574 (1988).

Barnes et al. Methods Enzymol. 101:98-122 (1983).

Bibb, M. J., Bibb, M. J., Ward, J. M., Cohen, S. N. Mol. Gen. Genet., 199:26-36 (1985).

Bierman, M., Logan, R., O'Brien, K., Seno, G., Nagaraja, R., Schoner, B. E. Gene, 116:43-49(1992).

Borisova, S. A., Zhao, L., Sherman, D. H., Liu, H.-w., Org. Lett., 1, 133-136 (1999).

Box, R. P. Clin. Infect. Dis., 24:S151 (1997).

Cane, D. E., Larnbalot, R. H., Prabhakaran, P. C., Ott, W. R. J. Am. Chem. Soc., 115:522-526 (1993).

Cane, D. E., Walsh, C. T., Khosla, C., Science, 282. 63-68 (1998).

Carreras, C. W., Pieper, R., Khosla, C. In Bioorganic Chemistry Deoxysugars, Polyketides & Related Classes: Synthesis, Biosynthesis, Enzymes, Rohr, J. (ed.), Springer:Berlin, 85-126 (1997).

Castle, L. A., Smith, K. D., Morris, R. O. J. Bacteriol., 174:1478-1486 (1992).

Celmer, W. D., Nagel, A. A., Wadlow, J. W., Tatematsu, H., Ikenaga, S., Nakanishi, S. Abstracts of Papers of 24th Intersci. Conf. on Antimicrob. Agents Chemother., No. 1142, Washington, D. C. (1985).

Chang et al., J. Am. Chem. Soc., 122:4239-4240 (2000).

Chen, H., Yeung, S.-M., Que, N. L. S., Müller, T., Schmidt, R. R., Liu, H.-w., J. Am. Chem. Soc., 121:8124(1999).

Clark et al. Proc. Natl. Acad. Sci. USA 53:451-459 (1965).

Cortes, J. Haydock, S. F., Roberts, G. A., Bevitt, D. J., Leadlay, P. F. Nature, 348:176-8 (1990).

Cortes, J., Wiesmann, K. E., Roberts, G. A., Brown, M. J., Staunton, J., Leadlay, P. F. Science, 268:1487-9 (1995).

Crameri et al., Nature, 391 288 (1998).

Cundliffe, E. C. Annu. Rev. Microbiol., 43:207-233 (1989).

Cundliffe, E. Antimicrob. Agents Chemother., 36:348-352 (1992).

Cundliffe, E., Self-Protection Mechanisms in Antibiotic Producers; Cundliffe, E., Ed., Wiley: Chichester, 199-214 (1992).

Dalbie-McFarland et al., Proc. Natl. Acad. Sci. USA 79:6409 (1982).

Davies, J. Nature, 383:219-220 (1996).

Dayhoff, M. O., in Atlas of Protein Sequence and Structure, 1972, volume 5, National Biomedical Research Foundation, pp.101-110, and Supplement 2 to this volume, pp. 1-10.

Decker, H., Haag, S., Udvamoki, G., Rohr, J., Angew. Chem. Int. Ed. (1995).

Denis, F., Brzezinski, R. Gene, 111:115-118 (1992).

Ding, W.-d., Ellestad, G. A., J. Am. Chem. Soc., 113. 6617 (1991).

Distler, J., Mansouri, K., Mayer, G., Stockmann, M., Piepersberg, W., Gene, 115, 105-111 (1992).

Djerassi, C., Zderic, J. A. J. Am. Chem. Soc., 78:6390-6395 (1956).

Donadio, S., McAlpine, J. B., Sheldon, P. J., Jackson, M., Katz, L. Proc. Natl. Acad. Sci. U.S.A., 90:7119-7123 (1993).

Donadio, S., Staver, M. J., McAlpine, J. B., Swanson, S. J., Katz, L. Science, 252:675-9 (1991).

Donadio, S., Katz, L. Gene, 111:51-60 (1992).

Donin, M. N., Pagano, J., Dutcher, J. D., McKee, C. M. Antibiotics Annu. 1:179-185 (1953-1954).

Draeger et al., J. Am. Chem. Soc., 121:2611-2612 (1999).

Drak, J., Iawasawa, S., Danishefsky, S., Crothers, D., Proc. Natl. Acad. Sci, 88, 7464 (1991).

Ellestad, G. A., Ding, W.-D., Zein, N., Townsend, C. A., In Calicheamicins: DNA-Cleaving Properties of Calicheamicin, Borders, D. B., Doyle, T. W., Eds.; Marcel Dekker: New York, 137-160 (1995).

Epp, J., Huber, M. L. B., Tuner, J. R., Goodson, T., Schoner, B. E. Gene, 85:293-301 (1989).

Ferreiro, M. J., Latypov, S. K., Quinoa, E., Riguera, R., J. Org. Chem., 65, 2658-2666 (1991).

Flinn, E. H., Sigal, M. V., Jr., Wiley, P. F., Gerzon, K. J. Am. Chem. Soc., 76:3121-3131 (1954).

Frey et al., Arch. Biochem. Biophys. 382: 6-14 (2000).

Frey, P. A., In Comprehensive Chemistry of Natural Products; Barton, D., Nakanishi, K., Meth-Cohn, O., Eds.; Pergamon: New York, 1999; Vol.5, pp. 205-223.

Gaisser, S., Bohm, G. A., Cortés, J., Leadlay, P. F. Mol. Gen. Genet., 256:239-251 (1997).

Gaisser, S., Bohm, G. A., Doumith, M., Raynal, M.-C., Dhillon, N., Cortés, J., Leadlay, P. F., Mol. Gen. Genet., 258 78-88 (1998).

Gandecha, A. R., Large, S. L., Cundliffe, E. Gene, 184:197-203 (1997).

Gebb, C., Baron, D., Grisebach, H., Eur. J. Biochem., 54, 493-498 (1975).

Geisselsoder et al. BioTechniques 5:786 (1987).

Geistlich, M., Losick, R., Turner, J. R., Rao, R. N. Mol. Microbiol., 6:2019-2029 (1992).

Gokhale, R. S., Hunziker, D., Cane, D. E., Khosla, C. Chem. Biol., 6:117-125 (1999).

Hallis et al., Acc. Chem. Res., 3: 579-588 (1999).

Haydock, S. F., Dowson, J. A., Dhillon, N.,. Roberts, G. A., Cortés, J., Leadlay, P. F. Mol. Gen. Genet., 230:120-128 (1991).

He et al., Chem. Rev., 100: 4615-4661 (2000).

Hernandez, C., Olano, C., Mendez, C., Salas J. A. Gene, 134:139-140 (1993).

Hopwood, D. A., Sherman, D. H. Annu. Rev. Genet., 24:37-66 (1990).

Hopwood, D. A., Malpartida, F., Kieser, H. M., Ikeda, H., Duncan, J., Fujii, I., Rudd, B. A., Floss, H. G., Omura, S. Nature, 314:642-644 (1985).

Hopwood, D. A., Bibb, M. J., Chater, K. J., Kieser, T., Bruton, C. J., Kieser, H. M., Lydiate, D. J., Smith, C. P., Ward, J. M., Schrempf, H., Genetic Manipulation of Streptomyces: A Laboratory Manual (The John Innes Foundation) (1985).

Hori et al., Chem. Comm., 304 (1971).

Hutchinson, C. R., Fujii, I. Annu. Rev. Microbiol., 49:201-238 (1995).

Hutchinson, C. R., Curr. Op. Micro., 1, 319-329 (1998).

Ingrosso, D., Fowler, A. V., Bleibaum, J., Clarke, S. J. Biol. Chem., 264:20130-20139 (1989).

Jacobsen, J. R., Hutchinson, C. R., Cane, D. E., Khosla, C. Science, 277:367-369 (1997).

Jenksius, G., Cundliffe, E. Gene, 108, 55-62 (1991).

Johnson et al., Biochemistry, 35:15846-15856 (1996).

Johnson et al., In The Biology-Chemistry Interface: A Tribute to Koji Nakanishi; Cooper, R., Snyder, J. D., Eds.; Marcel Dekker: New York; 1999; pp 351-396.

Kakavas, S. J., Katz, L., Stassi, D. J. Bacteriol., 179:7515-22 (1997).

Kao, C. M., Luo, G. L., Katz, L., Cane, D. E., Khosla, C. J. Am. Chem. Soc., 117:9105-9106 (1995).

Katz, L., Donadio, S. Annu. Rev. Microbiol., 47:875-912 (1993).

Katz, L., Chem. Rev., 97:2557-2575 (1997).

Kelleher, W. J., Baron, D., Ortmann, R., Grisebach, H., FEBS Lett., 22, 203-204 (1972).

Khosla, C., Chem. Rev., 97:2577-2590 (1997).

Khosla, C., Zawada, R. J. Trends Biotechnol., 14:335-341 (1996).

Kirschning, A., Bechthold, A. F.-W., Rohr, J. In Bioorganic Chemistry Deoxysugars, Polyketides & Related Classes: Synthesis, Biosynthesis, Enzymes, Rohr, J. (ed.), Springer:Berlin 1-84 (1997).

Kirschning et al., Top. Curr. Chem., 188:1-84 (1997).

Kramer, P. J., Khosla, C. Annu. N.Y. Acad. Sci., 799:32-45 (1996).

Kunkel, T. A. Proc. Natl. Acad. Sci. USA 82:448 (1985).

Kuo, M.-S., Chirby, D. G., Argoudelis, A. D., Cialdella, J. I., Coats, J. H., Marshall, V. P. Antimicrob. Agents Chemother., 33:2089-2091 (1989).

Lambalot, R. H., Cane, D. E. J. Antibiot., 45:1981-1982 (1992).

Lin, E. C. C., Goldstein, R., Syvanen, M. Bacteria, Plasmids, and Phages, An Introduction to Molecular Biology, Harvard University Press:Cambridge, p. 123 (1984).

Liu, H.-w., Thorson, J. S. Annu. Rev. Microbiol., 48:223-256 (1994).

LoBrutto et al., Biochemistry,, 40:9-14 (2001).

Lydiate, D. J., Malpartida, F., Hopwood, D. A. Gene, 35:223-235 (1985).

Madduri, K., Kennedy, J., Rivola, G., Inventi-Solari, A., Filippini, S., Zanuso, G., Colombo, A. L., Gewain, K. M., Occi, J. L., MacNeil, D. J., Hutchinson, C. R. Nature Biotech.. 16:69-74 (1998).

Maier, S., Matern, U., Grisebach, H., FEBS Lett., 49, 317-319 (1975).

Mangahas, F. R. MS Thesis, University of Minnesota, 1996.

Marahiel, M. A., Stachelhaus, T., Mootz, H. D., Chem. Rev. 97:2651-2673 (1997).

Marsden, A. F. A., Wilkinson, B., Cortés, J., Dunster, N. J., Staunton, J., Leadlay, P. F. Science, 279:199-201 (1998).

Matern, U., Grisebach, H., Eur. J. Biochem., 74 303-312 (1977).

McDaniel, R., Thamchaipenet, A., Gustafsson, C., Fu, H., Betlach, M., Betlach, M., Ashley, G., Proc. Natl. Acad. Sci. USA, 96, 1846-1851 (1999).

McManus, M. C., Am. J. Health-Syst. Pharm., 54 1420-1433 (1997).

Merson-Davies, L. A., Cundliffe, E. Mol. Microbiol., 13:349-355 (1994).

Merson-Davies, L. A., Cundliffe, E. Mol. Microbiol., 13:347-355 (1994).

Monaco and Lavin, Trends in Biotech., 12, 280 (1994).

Motamedi, H., Cai, S. J., Shafiee, A., Elliston, K. O. Eur. J. Biochem., 244:74-80 (1997).

Muller et al. Liebigs Ann., 1907-1914 (1997).

Muth, G., Nubhaumer, B., Wohlleben, W., Puhler, A. Mol. Gene. Genet., 219:341-348 (1989).

Needleman and Wunsch J. Mol. Biol. 48: 443 (1970).

Niemi, J., Mantsala, P. J. Bacteriol., 177:2942-2945 (1995).

Ohtani, I., Kusumi, T., Kashman, Y., Kakisawa, H., J. Am. Chem. Soc., 113, 4092-4096 (1991).

Omura, S. (ed.) Macrolide Antibiotics, Chemistry, Biology, and Practice, Academic Press:New York (1984).

Omuras et al., J. Antibio., 29, 316 (1971).

Ortmann, P., Matern, U., Grisebach, H., Stadler, P., Sinnwell, V., Paulsen, H., Eur. J. Biochem., 43, 265-271 (1974).

Patten et al., Curr. Op. Biotech., 8, 724 (1997).

Pearson and Lipman Proc. Natl. Acad. Sci. (U.S.A.) 85: 2444 (1988).

Pisowotzki, K., Mansori, K., Piepersberg, M., Mol. Gen. Genet., 231, 113-123 (1991).

Rangaswamy, V., Mitchell, R., Ullrich, M., Bender, C. J. Bacteriol., 180:3330-3338 (1998).

Russell et al. Gene, 45:333-338 (1986).

Ruzicka et al., J. Bacteriol. 182:469-476 (2000).

Salah-Bey, K., Doumith, M., Michel, J.-M., Haydock, S., Cortes, J., Leadlay, P. F., Raynal, M.-C., Mol. Gen. Genet., 257 542-553 (1998).

Sambrook, J., Fritsch, E. F., Maniatis, T. Molecular Cloning: A LaboratorM Manual (Cold Spring Harbor Laboratory Press), 2nd edition (1989).

Sasaki, J., Mizoue, K., Morimoto, S., Omura, S. J. Antibiotics, 49:1110-1118 (1996).

Schalkwyk et al., Curr. Op. Biotech., 6, 37 (1995).

Schneider, A., Marahiel, M. A., Arch. Microbiol., 169:404-410 (1998).

Schupp, T., Toupet, C., Cluzel, B., Neff, S., Hill, S., Beck, J. J., Ligon, J. M., J. Bacteriol., 177:3673-9 (1995).

Schwecke, T., Aparicio, J. F., Molnar, I., Konig, A., Khaw, L. E., Haydock, S. F., Oliynyk, M., Caffrey, P., Cortes, J., Lester, J. B., et al. Proc. Natl. Acad. Sci. U.S.A., 92:7839-7843 (1995).

Seo, S., Tomita, Y., Tori, K., Yoshimura, Y. J. Am. Chem. Soc. 100:3331-3339 (1978).

Service, R. F. Science, 270:724-727 (1995).

Smith and Waterman Adv. Appl. Math. 2: 482 (1981).

Sofia et al., Nucleic Acids Res., 29:1097-1106 (2001).

Solenberg, P. J., Matsushima, P., Stack, D. R., Wilkie, S. C., Thompson, R. C., Baltz, R. H., Chem. Biol., 4 195-202 (1997).

Stassi, D., Donadio, S., Staver, M. J., Katz, L. J. Bacteriol., 175:182-189 (1993).

Staunton, J., Caffrey, P., Aparicio, J. F., Roberts, G. A., Bethell, S. S., Leadlay, P. F. Nat. Struct. Biol., 3:188-192 (1996).

Staunton, J., Wilkinson, B., Chem. Rev., 97:2611-2629 (1997).

Summers, R. G., Donadio, S., Staver, M. J., Wendt-Pienkowski, E., Hutchinson, C. R., Katz, L. Microbiology, 143:3251-3262 (1997).

Swan, D. G., Rodriguez, A. M., Vilches, C., Mendez, C., Salas, J. A. Mol. Gen. Genet., 242:358-362 (1994).

Thorson, J. S., Shen, B., Whitwam, R. E., Liu, W., Li, Y., Ahlert, J., Bioorg. Chem., 27, 172-188 (1999).

Thorson et al., Am. Chem. Soc., 115:12177-12178 (1993). (b Tuan, J. S., Weber, J. M., Staver, M. J., Leung, J. O., Donadio, S., Katz, L. Gene 90:21-29 (1990).

Vilches, C., Hernandez, C., Mendez, C., Salas, J. A. J. Bacteriol., 174:161-165 (1992).

von Heijne, G. Nucleic Acids Res., 14:4683-4690 (1986).

von Heijne, G., Abrahmsen, L. FEBS Lett., 244:439-446 (1989).

Wahl, P., Matern, U., Grisebach, H., Biochem, Biophys, Res, Commun., 64, 1041-1045 (1975).

Wahl, H. P., Grisebach, H., Adv. Carbohydr, Chem. Biochem., 35, 81-126 (1978).

Wahl, H. P., Grisebach, H., Biochem. Biophys Acta, 568. 243-252 (1979).

Walker, S., Yang, D., Kahne, D., Gange, D., J. Am. Chem. Soc., 113, 4716-4717 (1991).

Watson, R. R., Orenstein, N. S., Adv. Carbohydr. Chem. Biochem., 31, 135-184 (1975).

Weber, J. M., Leung, J. O., Swanson, S. J., Idler, K. B., McAlpine, J. B. Science, 252:114-117 (1991).

Weigel et al., Biochemistry, 31:2129-2139 (1992).

Weymouth-Wilson, A. C., Nat. Prod. Rep., 14, 99-110 (1997).

Wohlert, S.-E., Blanco, G., Lombo, F., Fernandez, E., Brana, A. F., Reich, S., Udvarnoki, G., Mendez, C., Decker, H., Frevert, J., Salas, J. A., Rohr, J., J. Am. Chem. Soc., 120 10596-10601 (1998).

Xue, Y., Wilson, D., Zhao, L., Liu, H.-w., Sherman, D. H., Chem. Biol., 5 661-667 (1998).

Xue, Y., Zhao, L., Liu, H.-w., Sherman, D. H. Proc. Natl. Acad. Sci. U.S.A., 95: 12111-12116 (1998).

Xue, Y., Sherman, D. H., Nature. 403, 571-575 (2000).

Yu et al., J. Biol. Chem., 267:5868-5875 (1992).

Zhang and Wing, Plant Mol. Biol., 35, 115 (1997).

Zhao, L., Sherman, D. H., Liu, H.-w., J. Am. Chem. Soc., 120 9374-9375 (1998).

Zhao, L., Sherman, D. H., Liu, H.-w., J. Am. Chem. Soc., 120, 10256-10257 (1998).

Zhao, L., Que, N. L. S., Xue, Y., Sherman, D. H., Liu, H.-w., J. Am. Chem. Soc., 120, 12159-12160 (1998).

Zhao et al., J. Am. Chem. Soc., 121:9881-9882 (1999).

Zoller and Smith, Methods Enzymol., 100:468 (1983).

The complete disclosure of all patents, patent documents and publications cited herein are incorporated herein by reference as if individually incorporated. The foregoing detailed description and examples have been given for clarity of understanding only. No unnecessary limitations are to be understood therefrom. The invention is not limited to the exact details shown and described for variations obvious to one skilled in the art will be included within the invention defined by the claims.

What is claimed is:

1. A modified recombinant bacterial host cell which produces a product comprising a sugar that is not produced by the corresponding recombinant or nonrecombinant bacterial host cell, wherein the modified recombinant host cell and the recombinant host cell comprise a disruption in a nucleic acid sequence encoding at least one sugar biosynthetic enzyme, wherein the modified recombinant host cell comprises at least one nucleic acid segment which encodes at least one sugar biosynthetic enzyme that is a homolog of the enzyme encoded by the nucleic acid sequence, and wherein the sugar on the product produced by the modified recombinant host cell is not a stereoisomer of a sugar on the corresponding product of the recombinant or nonrecombinant host cell.

2. The modified recombinant host cell of claim 1 wherein the product produced by the modified recombinant host cell is a glycosylated polyketide.

3. The modified recombinant host cell of claim 2 wherein the product is a macrolide, anthracycline, angucycline, avermectin, milbemycin, tetracycline, polyene, polyether, ansamycin or isochromanequinone.

4. The modified recombinant host cell of claim 1 wherein the nucleic acid sequence which is disrupted encodes desosamine.

5. The modified recombinant host cell of claim 1 which is a Streptomyces.

6. The modified recombinant host cell of claim 1 wherein the nucleic acid segment is obtained from a cell that produces streptomycin, carbomycin, tylosin, spiramycin, streptothricin, erythromycin, vancomycin, teicoplanin, chloroeremycin, methymycin, pikromycin, uramycin, granaticin, oleandomicin, landomycin, tetracenomycin, doxorubicin, mithramycin, epirubicin, daunoribicin, calicheamicin or nystatin.

7. The modified recombinant host cell of claim 4 wherein the nucleic acid sequence encoding DesI or DesVIII is disrupted.

8. The modified recombinant host cell of claim 1 wherein the nucleic acid segment encodes a dehydrase.

9. The modified recombinant host cell of claim 1 wherein the nucleic acid segment encodes a reductase.

10. The modified recombinant host cell of claim 1 wherein the nucleic acid segment encodes a TDP-sugar synthase.

11. The modified recombinant host cell of claim 1 wherein the nucleic acid segment encodes a TDP-sugar-dehydratase.

12. The modified recombinant host cell of claim 1 wherein the nucleic acid segment encodes an aminotransferase.

13. The modified recombinant host cell of claim 1 wherein the nucleic acid segment encodes a N-methyltransferase.

14. The modified recombinant host cell of claim 1 wherein the nucleic acid segment encodes a tautomerase.

15. The modified recombinant host cell of claim 1 wherein the nucleic acid segment encodes an enzyme that is the homolog of the enzyme encoded by nucleic acid sequence.

16. The modified recombinant host cell of claim 1 which comprises at least two different nucleic acid segments.

17. The modified recombinant host cell of claim 16 wherein one of the nucleic acid segments encodes an epimerase.

18. The modified recombinant host cell of claim 16 wherein one of the nucleic acid segments encodes a dihydrostreptose synthase.

19. The modified recombinant host cell of claim 1 or 7 wherein the nucleic acid segment encodes CalH.

20. The modified recombinant host cell of claim 16 wherein the nucleic acid sequence encodes DesI and the nucleic acid segments encode StrL and StrM.

21. A product produced by the modified recombinant host cell of claim 1 which is not produced by the corresponding nonrecombinant or recombinant host cell.

22. The product of claim 21 which comprises a macrolide.

23. The product of claim 21 which is biologically active.

24. The product of claim 21 which is a polyketide.

25. A method to prepare a product having an altered sugar component, comprising: culturing the modified recombinant host cell of claim 1 so as to yield a product having an altered sugar component relative to the product produced by the corresponding nonrecombinant or recombinant host cell.

26. A method to identify a product produced by a modified recombinant host cell comprising:
a) introducing to a recombinant host cell at least one expression cassette so as to yield a modified recombinant host cell, wherein the recombinant host cell comprises a disruption in at least a portion of a nucleic acid sequence encoding at least one sugar biosynthetic enzyme, wherein the expression cassette comprises a nucleic acid segment which encodes a sugar biosynthetic enzyme that is different than the at least one enzyme encoded by the nucleic acid sequence; and
b) detecting whether the modified recombinant host cell produces a product that is different than a product produced by the recombinant host cell.

27. A method to prepare a modified recombinant host cell, comprising introducing to a recombinant host cell at least one expression cassette so as to yield a modified recombinant host cell, wherein the recombinant host cell comprises a disruption in at least a portion of a nucleic acid sequence encoding at least one sugar biosynthetic enzyme, wherein the expression cassette comprises a nucleic acid segment which encodes a sugar biosynthetic enzyme that is different than the at least one enzyme encoded by the nucleic acid sequence.
